(19) United States
(12) Patent Application Publication
(10) Pub. No.: US 2010/0247528 A1
(43) Pub. Date: Sep. 30, 2010

(54) ARRAYS, KITS AND CANCER CHARACTERIZATION METHODS

(76) Inventors: Kent Hunter, Potomac, MD (US); Nigel Crawford, N. Bethesda, MD (US)

Correspondence Address: LEYDIG, VOIT & MAYER, LTD., TWO PRUDENTIAL PLAZA, SUITE 4900, 180 NORTH STETSON AVENUE, CHICAGO, IL, 60601-6731, US

(21) Appl. No.: 12/676,693

(22) PCT Filed: Sep. 4, 2008
(86) PCT No.: PCT/US2008/075242

§ 371 Date: Mar. 24, 2010

Related U.S. Application Data
(60) Provisional application No. 60/970,400, filed on Sep. 6, 2007.

(51) Int. Cl. A61K 38/02; C40B 40/06; C40B 40/10; C40B 30/00; A61K 38/46; A61K 31/255; A61K 31/282; A61K 33/24; A61K 31/704; A61K 31/513; A61K 31/7068; A61K 31/17; A61K 31/4985; A61K 31/337; A61K 39/395; A61K 31/437; A61P 35/00

(52) U.S. Cl. 424/133.1; 506/16; 506/18; 506/7; 514/19.3; 424/94.6; 514/517; 514/492; 424/649; 514/34; 514/274; 514/49; 514/588; 514/249; 514/449; 514/281

(57) ABSTRACT

The invention provides an array comprising a substrate and a set of addressable elements, wherein each addressable element comprises (i) a polynucleotide that specifically binds to a target molecule, (ii) a polypeptide that specifically binds to target molecule, or (iii) a combination of (i) and (ii), wherein the target molecule is selected from the group of cancer-related target molecules as defined herein. Related kits, methods, and uses as described herein are further provided by the invention.

### CROSS-REFERENCE TO RELATED APPLICATIONS

This patent application claims the benefit of U.S. Provisional Patent Application No. 60/970,400, filed Sep. 6, 2007, which is incorporated by reference.

### BACKGROUND OF THE INVENTION

The process of metastasis is of great importance to the clinical management of cancer since the majority of cancer mortality is associated with metastatic disease rather than the primary tumor (Liotta et al., Principles of molecular cell biology of cancer: Cancer metastasis (4th ed.), Cancer: Principles & Practice of Oncology, ed. S. H. V. DeVita and S. A. Rosenberg, Philadelphia, Pa.: J. B. Lippincott Co., 134-149 (1993)). In most cases, cancer patients with localized tumors have significantly better prognoses than those with disseminated tumors. Since recent evidence suggests that the first stages of metastasis can be an early event (Schmidt-Kittler et al., Proc. Natl. Acad. Sci. U.S.A., 100 (13): 7737-7742 (2003)) and that 60-70% of patients have initiated the metastatic process by the time of diagnosis, a better understanding of the factors leading to tumor dissemination is of vital importance. However, even patients that have no evidence of tumor dissemination at primary diagnosis are at risk for metastatic disease. Approximately one-third of women who are sentinel lymph node negative at the time of surgical resection of the primary breast tumor will subsequently develop clinically detectable secondary tumors (Heimann et al., Cancer Res., 60 (2): 298-304 (2000)). Even patients with small primary tumors and node negative status (T1N0) at surgery have a significant chance (15-25%) of developing distant metastases (Heimann et al., J. Clin. Oncol., 18 (3): 591-599 (2000)). The foregoing shows that there is a need for a method of characterizing a tumor or a cancer in a subject, especially in terms of the metastatic capacity of a tumor.

### BRIEF SUMMARY OF THE INVENTION

The invention provides an array comprising a substrate and a set of addressable elements, wherein each addressable element comprises (i) a polynucleotide that specifically binds to a target molecule, (ii) a polypeptide that specifically binds to a target molecule, or (iii) a combination of (i) and (ii), wherein the target molecule is selected from the group of target molecules as defined herein, wherein the array comprises less than 38,500 addressable elements.

The invention also provides a kit comprising a set of user instructions and (i) a set of polynucleotides, (ii) a set of polypeptides, or (iii) a combination of (i) and (ii), wherein the set of polynucleotides is specific for one or more of the target molecules selected from the group of target molecules as defined herein, wherein the set of polypeptides is specific for the target molecules selected from the group as defined herein.

The invention further provides a method of characterizing a tumor or cancer in a subject comprising (i) detecting the expression levels of a set of target molecules in the subject and (ii) comparing the expression level of the set of target molecules to a control set of expression levels. In a first embodiment of the inventive method, the set of target molecules comprises one or more of the target molecules selected from the group as defined herein and the expression level is detected with the array or kit of the invention. In a second embodiment of the inventive method, the set of addressable elements consists essentially of the addressable elements that are specific for the target molecules described herein.

Further provided is the use of a compound with anti-cancer activity for the preparation of a medicament to treat cancer in a subject for whom the expression levels of a set of target molecules are determined. In a first embodiment of the inventive use, the set of target molecules comprises one or more of the target molecules described herein and the expression levels are determined with the array or kit of the invention. In a second embodiment of the inventive use, the set of addressable elements consists essentially of the addressable elements that are specific for the target molecules described herein.

### BRIEF DESCRIPTION OF THE SEVERAL VIEWS OF THE DRAWING(S)

FIG. 1A is a Kaplan Meier Curve of the Cox proportional analysis of the Mvt-1/Brd4 microarray gene expression signature on the GSE1456 breast cancer cohort in terms of overall survival.

FIG. 1B is a Kaplan Meier Curve of the Cox proportional analysis of the Mvt-1/Brd4 microarray gene expression signature on the GSE3494 breast cancer cohort in terms of overall survival.

FIG. 1C is a Kaplan Meier Curve of the Cox proportional analysis of the Mvt-1/Brd4 microarray gene expression signature on the GSE2034 breast cancer cohort in terms of overall survival.

FIG. 1D is a Kaplan Meier Curve of the Cox proportional analysis of the Mvt-1/Brd4 microarray gene expression signature on the GSE4922 breast cancer cohort in terms of overall survival.

FIG. 1E is a Kaplan Meier Curve of the Cox proportional analysis of the Mvt-1/Brd4 microarray gene expression signature on the Rosetta breast cancer cohort (van 't Veer et al., Nature 415: 530-536 (2002)) in terms of overall survival.

FIG. 1F is a Kaplan Meier Curve of the Cox proportional analysis of the van't Veer gene expression signature described in van't Veer et al., Nature 415: 530-536 (2002) on the Rosetta breast cancer cohort in terms of overall survival.

FIG. 2A is a Kaplan Meier Curve of the Cox proportional analysis of the Mvt-1/Brd4 microarray gene expression signature on the lymph node-negative patients of the GSE3494 breast cancer cohort.

FIG. 2B is a Kaplan Meier Curve of the Cox proportional analysis of the Mvt-1/Brd4 microarray gene expression signature on the lymph node-negative patients of the Rosetta breast cancer cohort.

FIG. 2C is a Kaplan Meier Curve of the Cox proportional analysis of the Mvt-1/Brd4 microarray gene expression signature on the lymph node-negative patients of the GSE2034 breast cancer cohort.

FIG. 2D is a Kaplan Meier Curve of the Cox proportional analysis of the Mvt-1/Brd4 microarray gene expression signature on the lymph node-negative patients of the GSE4922 breast cancer cohort.

FIG. 2E is a Kaplan Meier Curve of the Cox proportional analysis of the Mvt-1/Brd4 microarray gene expression signature on the estrogen receptor-positive patients of the GSE3494 breast cancer cohort.

FIG. 2F is a Kaplan Meier Curve of the Cox proportional analysis of the Mvt-1/Brd4 microarray gene expression signature on the estrogen receptor-positive patients of the Rosetta breast cancer cohort.

FIG. 2G is a Kaplan Meier Curve of the Cox proportional analysis of the Mvt-1/Brd4 microarray gene expression signature on the estrogen receptor-positive patients of the GSE2034 breast cancer cohort.

FIG. 2H is a Kaplan Meier Curve of the Cox proportional analysis of the Mvt-1/Brd4 microarray gene expression signature on the estrogen receptor-positive patients of the GSE4922 breast cancer cohort.

FIG. 3A is a Kaplan Meier Curve of the Cox proportional analysis of the Mvt-1/Anakin microarray gene expression signature on the GSE1456 breast cancer cohort in terms of overall survival.

FIG. 3B is a Kaplan Meier Curve of the Cox proportional analysis of the van't Veer 70-gene expression signature in terms of overall survival.

FIG. 3C is a Kaplan Meier Curve of the Cox proportional analysis of the Mvt-1/Anakin microarray gene expression signature on the lymph node-negative patients of the Dutch Rosetta breast cancer cohort.

FIG. 3D is a Kaplan Meier Curve of the Cox proportional analysis of the Mvt-1/Anakin microarray gene expression signature on the lymph node-positive patients of the Dutch Rosetta breast cancer cohort.

FIG. 3E is a Kaplan Meier Curve of the Cox proportional analysis of the Mvt-1/Anakin microarray gene expression signature on the estrogen receptor-positive patients of the Dutch Rosetta breast cancer cohort.

FIG. 3F is a Kaplan Meier Curve of the Cox proportional analysis of the van't Veer microarray gene expression signature on the estrogen receptor-negative patients of the Dutch Rosetta breast cancer cohort.

### DETAILED DESCRIPTION OF THE INVENTION

The invention provides arrays which can be used for detecting the expression levels of cancer-related target molecules. Each array comprises a substrate with which a set of addressable elements is associated in a predetermined manner. The array of the invention can, for example, be considered as a DNA chip, gene chip, or microarray.

As used herein, the term “addressable element” means an element that is attached to the substrate of the array at a predetermined position and specifically binds to a known target molecule, such that when target molecule-addressable element binding is detected, information regarding the identity of the bound target molecule is provided on the basis of the location of the element on the substrate. For the purposes of the invention, addressable elements are considered “different” if they do not bind to the same target molecule and/or the addressable elements are located at distinct positions within or on the substrate.

Generally, each of the addressable elements of the inventive arrays comprises a polynucleotide or polypeptide specific for (e.g., which specifically binds or hybridizes to) a target molecule. The polynucleotide or polypeptide may be referred to hereinafter as a “probe.” Generally, the probe is either a polynucleotide or polypeptide, depending on whether the target molecule for which the addressable element is specific is a polynucleotide or polypeptide. For example, if the target molecule is a nucleic acid target molecule (e.g., DNA, RNA, cDNA, etc.), and therefore is nucleotidic in nature, the addressable element can comprise a polynucleotide probe that specifically binds or hybridizes to the target molecule. Likewise, if the target molecule is a protein or polypeptide, the addressable element can comprise a polypeptide probe which specifically binds to the target molecule. However, the arrays of the invention are not so limited in this manner. The inventive arrays can, for example, comprise an addressable element comprising a polynucleotide which specifically binds to a polypeptide target molecule and/or comprise an addressable element comprising a polypeptide which binds to a polynucleotide target molecule.

Each of the addressable elements of the inventive arrays can independently comprise more than one copy of the polynucleotide or polypeptide probe. For instance, an addressable element can comprise multiple copies of a given polynucleotide or polypeptide probe having the same nucleotide or amino acid sequence. Additionally or alternatively, each of the addressable elements can independently comprise more than one different probe, provided that the probes selectively bind to the same target molecule. For example, an addressable element can comprise a first polynucleotide probe comprising a first sequence and a second polynucleotide probe comprising a second sequence which is different from the first sequence, wherein both the first and second probes bind to the same target molecule. Additionally or alternatively, an addressable element can comprise a polynucleotide probe and a polypeptide probe, each of which binds to the same target molecule.

In one embodiment of the invention, the array comprises a set of addressable elements, each of which comprises (i) a polynucleotide that specifically binds to a target molecule, (ii) a polypeptide that specifically binds to a target molecule, or (iii) a combination of (i) and (ii), wherein the target molecule is selected from the group consisting of the target molecules listed in Table 1.

TABLE 1

| | | | | Group(s) of |
|---|---|---|---|---|
| Target | Entrez | GenBank Accession No. | | which Target |
| Molecule Name | Gene ID No. | Nucleotide | Amino acid | Molecule is a Part |
| AARS | 16 | NM_001605.1 (SEQ ID NO: 7) | NP_001596.1 | 1, 2 |
| ALDH2 | 217 | NM_000690.2 (SEQ ID NO: 8) | NP_000681.2 (precursor) | 1 |
| ALDOC | 230 | NM_005165.2 (SEQ ID NO: 9) | NP_005156.1 | 1 |
| AQP1 | 358 | NM_198098.1 (SEQ ID NO: 10) | NP_932766.1 | 2 |
| ARHGEF6 | 9459 | NM_004840.2 (SEQ ID NO: 11) | NP_004831.1 | 1 |
| B4GALT6 | 9331 | NM_004775.2 (SEQ ID NO: 12) | NP_004766.1 | 1 |
| BYSL | 705 | NM_004053.3 (SEQ ID NO: 13) | NP_004044.3 | 2 |
| CELSR1 | 9620 | NM_014246.1 (SEQ ID NO: 14) | NP_055061.1 | 1 |
| CIRBP | 1153 | NM_001280.1 (SEQ ID NO: 15) | NP_001271.1 | 1, 2 |
| CLCN3 | 1182 | NM_173872.2 (SEQ ID NO: 16) | NP_776297.2 | 1 |
| | | NM_001829.2 | NP_001820.2 | |
| CRYAB | 1410 | NM_001885.1 (SEQ ID NO: 17) | NP_001876.1 | 1 |
| CTSO | 1519 | NM_001334.2 (SEQ ID NO: 18) | NP_001325.1 | 3 |
| DCTN6 | 10671 | NM_006571.2 (SEQ ID NO: 19) | NP_006562.1 | 3 |
| DDIT3 | 1649 | NM_004083.4 (SEQ ID NO: 20) | NP_004074.2 | 1 |
| DDX39 | 10212 | NM_005804.2 (SEQ ID NO: 21) | NP_005795.2 | 2, 4 |
| DKFZp564I0463 | — | AL117599 (SEQ ID NO: 22) | | 1 |
| FADS1 | 3992 | NM_013402.3 (SEQ ID NO: 23) | NP_037534.2 | 1 |
| FUT4 | 2526 | NM_002033.2 (SEQ ID NO: 24) | NP_002024.1 | 1 |
| FZD1 | 8321 | NM_003505.1 (SEQ ID NO: 25) | NP_003496.1 | 1, 3 |
| GLRB | 2743 | NM_000824.2 (SEQ ID NO: 26) | NP_000815.1 | 1 |
| GNG11 | 2791 | NM_004126.3 (SEQ ID NO: 27) | NP_004117.1 (precursor) | 1 |
| GNPAT | 8443 | NM_014236.1 (SEQ ID NO: 28) | NP_055051.1 | 1 |
| HBP1 | 26959 | NM_012257.3 (SEQ ID NO: 29) | NP_036389.2 | 1 |
| HOXB5 | 3215 | NM_002147.3 (SEQ ID NO: 30) | NP_002138.1 | 1 |
| IFRD1 | 3475 | NM_001007245.1 (SEQ ID NO: 31) | NP_001007246.1 | 1 |
| | | NM_001550.2 | NP_001541.2 | |
| IL13RA1 | 3597 | NM_001560.2 (SEQ ID NO: 32) | NP_001551.1 | 1 |
| JAK1 | 3716 | NM_002227.1 (SEQ ID NO: 33) | NP_002218.1 | 2 |
| LAMP2 | 3920 | NM_002294.1 | NP_002285.1 (precursor) | 1 |
| | | NM_013995.1 (SEQ ID NO: 34) | NP_054701.1 (precursor) | |
| LCP1 | 3936 | NM_002298.2 (SEQ ID NO: 35) | NP_002289.1 | 1 |
| LRRC16 | 55604 | NM_017640.3 (SEQ ID NO: 36) | NP_060110.3 | 3 |
| MCCC1 | 56922 | NM_020166.2 (SEQ ID NO: 37) | NP_064551.2 | 1 |
| MCCC2 | 64087 | NM_022132.3 (SEQ ID NO: 38) | NP_071415.1 | 1 |
| MPDZ | 8777 | NM_003829.1 (SEQ ID NO: 39) | NP_003820.1 | 2 |
| NUP93 | 9688 | NM_014669.2 (SEQ ID NO: 40) | NP_055484.2 | 2 |
| PDCD4 | 27250 | NM_145341.2 (SEQ ID NO: 41) | NP_663314.1 (isoform 2) | 1 |
| | | NM_014456.3 | NP_055271.2 (isoform 1) | |
| PDF | 64146 | NM_022341.1 (SEQ ID NO: 42) | NP_071736.1 | 2 |
| PER2 | 8864 | NM_022817.1 (SEQ ID NO: 43) | NP_073728.1 (isoform 1) | 1, 2 |
| | | NM_003894.3 | NP_003885.2 (isoform 2) | |
| PLAT | 5327 | NM_033011.1 | NP_127509.1 (isoform 3) | 1 |
| | | NM_000931.2 | NP_000922.2 (isoform 2) | |
| | | NM_000930.2 (SEQ ID NO: 44) | NP_000921.1 (isoform 1 | |
| | | | preprotein) | |
| PPAP2B | 8613 | NM_003713.3 (SEQ ID NO: 45) | NP_003704.3 | 2 |
| | | NM_177414.1 | NP_803133.1 | |
| RAB6B | 51560 | NM_016577.2 (SEQ ID NO: 46) | NP_057661.2 | 1 |
| SAP30 | 8819 | NM_003864.3 (SEQ ID NO: 47) | NP_003855.1 | 3, 4 |
| SLC16A3 | 9123 | NM_004207.1 (SEQ ID NO: 48) | NP_004198.1 | 1, 3, 4 |
| SLC19A1 | 6573 | NM_194255.1 | NP_919231.1 (isoform a) | 1 |
| | | NM_003056.2 (SEQ ID NO: 49) | NP_003047.2 (isoform b) | |
| SMARCA2 | 6595 | NM_003070.3 (SEQ ID NO: 50) | NP_003061.3 (isoform a) | 2 |
| | | NM_139045.2 | NP_620614.2 (isoform b) | |
| SNN | 8303 | NM_003498.4 (SEQ ID NO: 51) | NP_003489.1 | 1 |
| SORBS1 | 10580 | NM_015385.2 | NP_056200.1 (isoform 2) | 2 |
| | | NM_024991.1 | NP_079267.1 (isoform 6) | |
| | | NM_006434.2 | NP_006425.2 (isoform 1) | |
| | | NM_001034957.1 | NP_001030129.1 (isoform 7) | |
| | | NM_001034955.1 | NP_001030127.1 (isoform 4) | |
| | | NM_001034954.1 (SEQ ID NO: 52) | NP_001030126.1 (isoform 3) | |
| | | NM_001034956.1 | NP_001030128.1 (isoform 5) | |
| TFRC | 7037 | NM_003234.1 (SEQ ID NO: 53) | NP_003225.1 | 1 |
| TNS1 | 7145 | NM_022648.3 (SEQ ID NO: 54) | NP_072174.3 | 2, 3 |
| WDR26 | 80232 | NM_025160.4 (SEQ ID NO: 55) | NP_079436.3 | 2 |

The expression level of each of the target molecules of Table 1 significantly changes in cells when the cells overexpress the Anakin gene (also known in the art as Ribosomal RNA Processing 1 Homolog (RRP1B), which gene encodes the mRNA sequence of Accession No. NM_{—}015056 (SEQ ID NO: 1) and encodes the amino acid sequence of Accession No. NP_{—}0055871 (SEQ ID NO: 2), both sequences of which are available herein and from the GenBank database of the National Center for Biotechnology Information (NCBI) website. Ectopic expression of Anakin reduces tumor growth and metastasis burden in the highly metastatic Mvt-1 cell line. Therefore, the expression levels of the target molecules of Table 1 are characteristic of a tumor or a cancer in a subject, e.g., are predictive of whether a subject afflicted with cancer, e.g., breast cancer, will survive, as further described herein.

In a preferred embodiment of the invention, the array comprises a set of addressable elements, such that the set comprises an addressable element specific for each of the target molecules of Table 1. In this regard, all of the target molecules of Table 1 are detected by the array. Alternatively or additionally, the set of addressable elements can consist essentially of addressable elements specific for cancer-related target molecules, as described herein, such that cancer-related target molecules are predominantly detected by the array. For example, the set of addressable elements can consist essentially of the addressable elements that are specific for the target molecules of Table 1, in combination with one or more addressable elements not listed in Table 1, e.g., a cancer-related target molecule (e.g., any of the target molecules listed in Table 2). Alternatively, the set can consist essentially of the addressable elements specific for the target molecules of Table 1.

As shown in Table 1, the target molecules of Table 1 are subdivided into different groups. The target molecules of Group 1 are target molecules of Table 1 which exhibit the same expression patterns (e.g., are either upregulated or downregulated in the same manner) in patients of the van 't Veer breast cancer cohort (van't Veer et al., Nature 415: 484-485 (2002)). Therefore, the expression levels of the target molecules of Group 1 are characteristic of a tumor or a cancer in a subject, e.g., are predictive of whether a subject afflicted with cancer, e.g., breast cancer, will survive, as described herein, especially if the tumor or cancer of the subject is similar to the tumor or cancer of the patients of the van't Veer breast cancer cohort.

The target molecules of Group 2 are target molecules of Table 1 which exhibit the same expression patterns (e.g., are either upregulated or downregulated in the same manner) in patients of the GSE1456 breast cancer cohort (Pawitan et al., Breast Cancer Res. 7: R953-R964 (2005)). Therefore, the expression levels of the target molecules of Group 2 are characteristic of a tumor or a cancer in a subject, e.g., are predictive of whether a subject afflicted with cancer, e.g., breast cancer, will survive, as described herein, especially if the tumor or cancer of the subject is similar to the tumor or cancer of the patients of the GSE1456 breast cancer cohort.

The target molecules of Group 3 are target molecules of Table 1 which exhibit the same expression patterns (e.g., are either upregulated or downregulated in the same manner) in patients of the GSE3494 breast cancer cohort (Miller et al., Proc. Natl. Acad. Sci. U.S.A. 102: 13550-13555 (2005)). Therefore, the expression levels of the target molecules of Group 3are characteristic of a tumor or a cancer in a subject, e.g., are predictive of whether a subject afflicted with cancer, e.g., breast cancer, will survive, as described herein, especially if the tumor or cancer of the subject is similar to the tumor or cancer of the patients of the GSE3494 breast cancer cohort.

The target molecules of Group 4 are target molecules of Table 1 which exhibit the same expression patterns (e.g., are either upregulated or downregulated in the same manner) in patients of the GSE4922 breast cancer cohort (Ivshina et al., Cancer Res. 66: 10292-10301 (2006)). Therefore, the expression levels of the target molecules of Group 4 are characteristic of a tumor or a cancer in a subject, e.g., are predictive of whether a subject afflicted with cancer, e.g., breast cancer, will survive, as described herein, especially if the tumor or cancer of the subject is similar to the tumor or cancer of the patients of the GSE4922 breast cancer cohort.

In one embodiment of the invention, the array comprises a set of addressable elements specific for the target molecules listed in Group 1, Group 2, Group 3, Group 4, or any combination thereof (e.g., Groups 1-4, Groups 1-3, Groups 1 and 2, Groups 2-4, Groups 2 and 3, Groups 3 and 4).

In a preferred embodiment of the invention, the array comprises a set of addressable elements, such that the set comprises an addressable element specific for each of the target molecules of the Group(s). In this regard, all of the target molecules of the Group(s) are detected by the array. Alternatively or additionally, the set of addressable elements can consist essentially of addressable elements specific for cancer-related target molecules, as described herein, such that cancer-related target molecules are predominantly detected by the array. For example, the set of addressable elements can consist essentially of the addressable elements that are specific for the target molecules of the Group(s), in combination with one or more addressable elements not listed in the Group(s), e.g., a cancer-related target molecule (e.g., any of the target molecules listed in any of the other Group(s), Table 2, or a combination thereof). Alternatively, the set can consist essentially of the addressable elements specific for the target molecules of the Group(s).

The array of the invention can additionally or alternatively comprise a substrate and a set of addressable elements, wherein each addressable element comprises (i) a polynucleotide that specifically binds to a target molecule, (ii) a polypeptide that specifically binds to a target molecule, or (iii) a combination of (i) and (ii), wherein the target molecule is selected from the group consisting of the target molecules listed in Table 2.

TABLE 2

| Target | Entrez | | | Group(s) of |
|---|---|---|---|---|
| Molecule | Gene ID | GenBank Accession No. | | which Target |
| Name | No. | Nucleotide | Amino acid | Molecule is a Part |
| ANLN | 54443 | NM_018685 (SEQ ID NO: 56) | NP_061155 | 5 |
| ASF1B | 55723 | NM_018154.2 (SEQ ID NO: 57) | NP_060624.1 | 6, 8, 9 |
| ASPM | 259266 | NM_018136.2 (SEQ ID NO: 58) | NP_060606.2 | 6 to 9 |
| ATF3 | 467 | NM_001030287.1 | NP_001025458.1 (isoform 1) | 7 |
| | | NM_001674.2 | NP_001665.1 (isoform 1) | |
| | | NM_004024.3 (SEQ ID NO: 59) | NP_004015.3 (isoform 2) | |
| AURKA | 6790 | NM_003600.2 | NP_003591.2 | 6 to 9 |
| | | NM_198433.1 (SEQ ID NO: 60) | NP_940835.1 | |
| | | NM_198435.1 | NP_940837.1 | |
| | | NM_198434.1 | NP_940836.1 | |
| | | NM_198437.1 | NP_940839.1 | |
| | | NM_198436.1 | NP_940838.1 | |
| AURKB | 9212 | NM_004217.2 (SEQ ID NO: 61) | NP_004208.2 | 6, 8, 9 |
| BIRC5 | 332 | NM_001012271.1 (SEQ ID NO: 62) | NP_001012271.1 (isoform 3) | 5 to 9 |
| | | NM_001168.2 | NP_001159.2 (isoform 1) | |
| | | NM_001012270.1 | NP_001012270.1 (isoform 2) | |
| BLM | 641 | NM_000057.1 (SEQ ID NO: 63) | NP_000048.1 | 5, 8 |
| BRCA1 | 672 | NM_007297.2 | NP_009228.1 (isoform BRCA1-delta2-10) | 7 |
| | | NM_007298.2 | NP_009229.1 (isoform BRCA1-delta9-11) | |
| | | NM_007302.2 | NP_009233.1 (isoform BRCA1-delta9-10) | |
| | | NM_007305.2 | NP_009236.1 (isoform BRCA1-delta9-10-11b) | |
| | | NM_007303.2 | NP_009234.1 (isoform BRCA1-delta11) | |
| | | NM_007300.2 | NP_009231.1 (isoform BRCA1-delta14-18) | |
| | | NM_007299.2 | NP_009230.1 (isoform BRCA1-delta14-17) | |
| | | NM_007294.2 | NP_009225.1 (isoform 1) | |
| | | NM_007304.2 | NP_009235.2 (isoform BRCA1-delta11b) | |
| | | NM_007296.2 | NP_009227.1 (isoform 1) | |
| | | NM_007295.2 (SEQ ID NO: 64) | NP_009226.1 (isoform 1) | |
| BRRN1 | 679 | NM_015341.3 (SEQ ID NO: 65) | NP_056156.2 | 6 to 9 |
| BUB1 | 699 | NM_004336.2 (SEQ ID NO: 66) | NP_004327.1 | 5 to 9 |
| BUB1B | 701 | NM_001211.4 (SEQ ID NO: 67) | NP_001202.4 | 5, 6, 8, 9 |
| C1S | 716 | NM_201442.1 (SEQ ID NO: 68) | NP_958850.1 | 6, 8, 9 |
| | | NM_001734.2 | NP_001725.1 | |
| CAD | 790 | NM_004341.3 (SEQ ID NO: 69) | NP_004332.2 | 5 |
| CASP3 | 836 | NM_032991.2 | NP_116786.1 (preproprotein) | 8, 9 |
| | | NM_004346.3 (SEQ ID NO: 70) | NP_004337.2 (preproprotein) | |
| CBL | 867 | NM_005188.2 (SEQ ID NO: 71) | NP_005179.2 | 5 |
| CCNA2 | 890 | NM_001237.2 (SEQ ID NO: 72) | NP_001228.1 | 5 to 9 |
| CCNB1 | 891 | NM_031966.2 (SEQ ID NO: 73) | NP_114172.1 | 5, 6, 8, 9 |
| CCNB2 | 9133 | NM_004701.2 (SEQ ID NO: 74) | NP_004692.1 | 5 to 9 |
| CCNE2 | 9134 | NM_057749.1 (SEQ ID NO: 75) | NP_477097.1 (isoform 1) | 5 to 9 |
| | | NM_057735.1 | NP_477083.1 (isoform 2) | |
| CDC20 | 991 | NM_001255.1 (SEQ ID NO: 76) | NP_001246.1 | 5 to 9 |
| CDC25B | 994 | NM_021873.2 (SEQ ID NO: 77) | NP_068659.1 (isoform 1) | 5, 6, 8, 9 |
| | | NM_021872.2 | NP_068658.1 (isoform 3) | |
| | | NM_004358.3 | NP_004349.1 (isoform 2) | |
| CDC25C | 995 | NM_022809.1 | NP_073720.1 (isoform b) | 5 |
| | | NM_001790.2 (SEQ ID NO: 78) | NP_001781.1 (isoform a) | |
| CDC45L | 8318 | NM_003504.3 (SEQ ID NO: 79) | NP_003495.1 | 5, 6, 8, 9 |
| CDC6 | 990 | NM_001254.3 (SEQ ID NO: 80) | NP_001245.1 | 5, 6, 9 |
| CDC7 | 8317 | NM_003503.2 (SEQ ID NO: 81) | NP_003494.1 | 6 |
| CDCA3 | 83461 | NM_031299.3 (SEQ ID NO: 82) | NP_112589.1 | 6, 7 |
| CDCA8 | 55143 | NM_018101.2 (SEQ ID NO: 83) | NP_060571.1 | 6 to 9 |
| CDKN2D | 1032 | NM_079421.2 | NP_524145.1 | 5 |
| | | NM_001800.3 (SEQ ID NO: 84) | NP_001791.1 | |
| CDKN3 | 1033 | NM_005192.2 (SEQ ID NO: 85) | NP_005183.2 | 5, 6, 8, 9 |
| CENPA | 1058 | NM_001809.2 (SEQ ID NO: 86) | NP_001800.1 (isoform a) | 5 to 9 |
| CENPE | 1062 | NM_001813.2 (SEQ ID NO: 87) | NP_001804.2 | 5 to 9 |
| CENPF | 1063 | NM_016343.3 (SEQ ID NO: 88) | NP_057427.3 | 5, 6, 8, 9 |
| CHEK1 | 1111 | NM_001274.2 (SEQ ID NO: 89) | NP_001265.1 | 5, 6, 9 |
| FOXN3 | 1112 | NM_005197.2 (SEQ ID NO: 90) | NP_005188.2 | 6, 8, 9 |
| (CHES1) | | | | |
| CHKA | 1119 | NM_212469.1 | NP_997634.1 (isoform b) | 6 |
| | | NM_001277.2 (SEQ ID NO: 91) | NP_001268.2 (isoform a) | |
| CIRBP | 1153 | NM_001280.1 (SEQ ID NO: 92) | NP_001271.15, | 5, 6, 8, 9 |
| CKAP2 | 26586 | NM_018204.2 (SEQ ID NO: 93) | NP_060674.2 | 5, 8, 9 |
| CKS2 | 1164 | NM_001827.1 (SEQ ID NO: 94) | NP_001818.1 | 5, 6, 8, 9 |
| CP | 1356 | NM_000096.1 (SEQ ID NO: 95) | NP_000087.1 | 5 |
| DCTD | 1635 | NM_001012732.1 (SEQ ID NO: 96) | NP_001012750.1 (isoform a) | 8 |
| | | NM_001921.2 | NP_001912.2 (isoform b) | |
| DDIT4 | 54541 | NM_019058.2 (SEQ ID NO: 97) | NP_061931.1 | 8, 9 |
| DHODH | 1723 | NM_001361.3 (SEQ ID NO: 98) | NP_001352.2 | 5 |
| | | NM_001025193.1 | NP_001020364.1 | |
| DIXDC1 | 85458 | NM_001037954.1 (SEQ ID NO: 99) | NP_001033043.1 (isoform a) | 6, 8 |
| | | NM_033425.2 | NP_219493.1 (isoform b) | |
| DLEU2 | 8847 | NR_002612 (SEQ ID NO: 100) | | 5 |
| DLG7 | 9787 | NM_014750.3 (SEQ ID NO: 101) | NP_055565.2 | 6 to 9 |
| DNA2L | 1763 | XM_166103.7 (SEQ ID NO: 102) | XP_166103.4 | 5, 8, 9 |
| ESPL1 | 9700 | NM_012291.3 (SEQ ID NO: 103) | NP_036423.3 | 6, 8, 9 |
| ETV5 | 2119 | NM_004454.1 (SEQ ID NO: 104) | NP_004445.1 | 7 |
| EXO1 | 9156 | NM_130398.2 (SEQ ID NO: 105) | NP_569082.1 (isoform b) | 5, 6 |
| | | NM_006027.3 | NP_006018.3 (isoform b) | |
| | | NM_003686.3 | NP_003677.3 (isoform a) | |
| EYA2 | 2139 | NM_005244.3 | NP_005235.3 (isoform a) | 6 |
| | | NM_172110.1 | NP_742108.1 (isoform c) | |
| | | NM_172113.1 (SEQ ID NO: 106) | NP_742111.1 (isoform b) | |
| | | NM_172111.1 | NP_742109.1 (isoform a) | |
| | | NM_172112.1 | NP_742110.1 (isoform a) | |
| EZH2 | 2146 | NM_152998.1 | NP_694543.1 (isoform b) | 5, 6, 7, 9 |
| | | NM_004456.3 (SEQ ID NO: 107) | NP_004447.2 (isoform a) | |
| FAS | 355 | NM_000043.3 (SEQ ID NO: 108) | NP_000034.1 (isoform 1 precursor) | 6 to 9 |
| | | NM_152872.1 | NP_690611.1 (isoform 3 precursor) | |
| | | NM_152871.1 | NP_690610.1 (isoform 2 precursor) | |
| | | NM_152873.1 | NP_690612.1 (isoform 4 precursor) | |
| | | NM_152874.1 | NP_690613.1 (isoform 4 precursor) | |
| | | NM_152875.1 | NP_690614.1 (isoform 5 precursor) | |
| | | NM_152877.1 | NP_690616.1 (isoform 7 precursor) | |
| | | NM_152876.1 | NP_690615.1 (isoform 6 precursor) | |
| FBXO5 | 26271 | NM_012177.2 (SEQ ID NO: 109) | NP_036309.1 | 6, 8, 9 |
| FEN1 | 2237 | NM_004111.4 (SEQ ID NO: 110) | NP_004102.1 | 5, 6, 8, 9 |
| FIGNL1 | 63979 | NM_022116.2 (SEQ ID NO: 111) | NP_071399.2 | 5 |
| FOS | 2353 | NM_005252.2 (SEQ ID NO: 112) | NP_005243.1 | 5, 8, 9 |
| FXYD5 | 53827 | NM_144779.1 (SEQ ID NO: 113) | NP_659003.1 | 5 |
| | | NM_014164.4 | NP_054883.3 | |
| GADD45A | 1647 | NM_001924.2 (SEQ ID NO: 114) | NP_001915.1 | 8 |
| GATM | 2628 | NM_001482.1 (SEQ ID NO: 115) | NP_001473.1 | 6, 8, 9 |
| GHR | 2690 | NM_000163.2 (SEQ ID NO: 116) | NP_000154.1 (precursor) | 6, 8 |
| GNAQ | 2776 | NM_002072.2 (SEQ ID NO: 117) | NP_002063.2 | 6 |
| GPR126 | 57211 | NM_020455.4 (SEQ ID NO: 118) | NP_065188.4 (alpha 1) | 9 |
| | | NM_198569.1 | NP_940971.1 (beta 1) | |
| | | NM_001032394.1 | NP_001027566.1 (alpha 2) | |
| | | NM_001032395.1 | NP_001027567.1 (beta 2) | |
| H6PD | 9563 | NM_004285.3 (SEQ ID NO: 119) | NP_004276.2 | 5 |
| HIST1H1C | 3006 | NM_005319.3 (SEQ ID NO: 120) | NP_005310.1 | 6, 8, 9 |
| HMGA1 | 3159 | NM_145899.1 | NP_665906.1 (isoform a) | 6 to 9 |
| | | NM_002131.2 | NP_002122.1 (isoform b) | |
| | | NM_145903.1 | NP_665910.1 (isoform b) | |
| | | NM_145901.1 | NP_665908.1 (isoform a) | |
| | | NM_145902.1 | NP_665909.1 (isoform b) | |
| | | NM_145904.1 (SEQ ID NO: 121) | NP_665911.1 (isoform a) | |
| | | NM_145905.1 | NP_665912.1 (isoform b) | |
| HMGB2 | 3148 | NM_002129.2 (SEQ ID NO: 122) | NP_002120.1 | 8, 9 |
| HMMR | 3161 | NM_012484.1 (SEQ ID NO: 123) | NP_036616.1 (isoform a) | 5 to 9 |
| | | NM_012485.1 | NP_036617.1 (isoform b) | |
| HSPA4L | 22824 | NM_014278.2 (SEQ ID NO: 124) | NP_055093.2 | 6 |
| ITGB5 | 3693 | NM_002213.3 (SEQ ID NO: 125) | NP_002204.2 | 5, 6, 8 |
| KIF11 | 3832 | NM_004523.2 (SEQ ID NO: 126) | NP_004514.2 | 6, 8, 9 |
| KIF18A | 81930 | NM_031217.2 (SEQ ID NO: 127) | NP_112494.2 | 8, 9 |
| KIF20A | 10112 | NM_005733.1 (SEQ ID NO: 128) | NP_005724.1 | 6, 8, 9 |
| KIF22 | 3835 | NM_007317.1 (SEQ ID NO: 129) | NP_015556.1 | 6 |
| KIF23 | 9493 | NM_138555.1 (SEQ ID NO: 130) | NP_612565.1 (isoform 1) | 6 to 9 |
| | | NM_004856.4 | NP_004847.2 (isoform 2) | |
| KIF2C | 11004 | NM_006845.2 (SEQ ID NO: 131) | NP_006836.1 | 6, 8, 9 |
| NDC80 | 10403 | NM_006101.1 (SEQ ID NO: 132) | NP_006092.1 | 8, 9 |
| (KNTC2) | | | | |
| KPNA2 | 3838 | NM_002266.2 (SEQ ID NO: 298) | NP_002257.1 | 6, 8, 9 |
| LAMP2 | 3920 | NM_002294.1 (SEQ ID NO: 133) | NP_002285.1 (precursor) | 5, 6 |
| | | NM_013995.1 | NP_054701.1 (precursor) | |
| LAT2 | 7462 | NM_022040.3 (SEQ ID NO: 134) | NP_071323.1 | 8 |
| | | NM_032463.2 | NP_115852.1 | |
| | | NM_014146.3 | NP_054865.2 | |
| LIG1 | 3978 | NM_000234.1 (SEQ ID NO: 135) | NP_000225.1 | 6, 8, 9 |
| LIPG | 9388 | NM_006033.2 (SEQ ID NO: 136) | NP_006024.1 | 5 |
| LRP8 | 7804 | NM_033300.2 | NP_150643.2 | 5 |
| | | NM_017522.3 | NP_059992.3 | |
| | | NM_001018054.1 | NP_001018064.1 | |
| | | NM_004631.3 (SEQ ID NO: 137) | NP_004622.2 | |
| LSM4 | 25804 | NM_012321.2 (SEQ ID NO: 138) | NP_036453.1 | 5, 6, 8, 9 |
| NCAPG2 | 54892 | NM_017760.4 (SEQ ID NO: 139) | NP_060230.4 | 6, 8, 9 |
| (LUZP5) | | | | |
| MAD2L1 | 4085 | NM_002358.2 (SEQ ID NO: 140) | NP_002349.1 | 5 to 9 |
| MCM3 | 4172 | NM_002388.3 (SEQ ID NO: 141) | NP_002379.2 | 5, 6, 8, 9 |
| MCM4 | 4173 | NM_005914.2 (SEQ ID NO: 142) | NP_005905.2 | 6, 8, 9 |
| | | NM_182746.1 | NP_877423.1 | |
| MCM5 | 4174 | NM_006739.2 (SEQ ID NO: 143) | NP_006730.2 | 5, 6, 8, 9 |
| MCM6 | 4175 | NM_005915.4 (SEQ ID NO: 144) | NP_005906.2 | 5, 6, 8, 9 |
| MELK | 9833 | NM_014791.2 (SEQ ID NO: 145) | NP_055606.1 | 6 to 9 |
| MKI67 | 4288 | NM_002417.2 (SEQ ID NO: 146) | NP_002408.2 | 5 to 9 |
| MLF1IP | 79682 | NM_024629.2 (SEQ ID NO: 147) | NP_078905.2 | 6, 8, 9 |
| MRE11A | 4361 | NM_005590.3 (SEQ ID NO: 148) | NP_005581.2 (isoform 2) | 7 |
| | | NM_005591.3 | NP_005582.1 (isoform 1) | |
| MTM1 | 4534 | NM_000252.1 (SEQ ID NO: 149) | NP_000243.1 | 5, 7 |
| MXRA8 | 54587 | NM_032348.2 (SEQ ID NO: 150) | NP_115724.1 | 6, 8 |
| NEDD4L | 23327 | NM_015277.2 (SEQ ID NO: 151) | NP_056092.2 | 6 |
| NEK2 | 4751 | NM_002497.2 (SEQ ID NO: 152) | NP_002488.1 | 5 to 9 |
| NFIL3 | 4783 | NM_005384.2 (SEQ ID NO: 153) | NP_005375.2 | 5 |
| NME1 | 4830 | NM_198175.1 (SEQ ID NO: 154) | NP_937818.1 (isoform a) | 6, 9 |
| | | NM_000269.2 | NP_000260.1 (isoform b) | |
| NOV | 4856 | NM_002514.2 (SEQ ID NO: 155) | NP_002505.1 (precursor) | 8, 9 |
| NUP205 | 23165 | NM_015135.1 (SEQ ID NO: 156) | NP_055950.1 | 6 |
| NUP93 | 9688 | NM_014669.2 (SEQ ID NO: 157) | NP_055484.2 | 6, 8, 9 |
| NUSAP1 | 51203 | NM_016359.2 (SEQ ID NO: 158) | NP_057443.1 (isoform 1) | 6, 8, 9 |
| | | NM_018454.5 | NP_060924.4 (isoform 2) | |
| OGN | 4969 | NM_033014.1 (SEQ ID NO: 159) | NP_148935.1 (preproprotein) | 5, 6 |
| | | NM_024416.2 | NP_077727.1 (preproprotein) | |
| | | NM_014057.2 | NP_054776.1 (preproprotein) | |
| PBK | 55872 | NM_018492.2 (SEQ ID NO: 160) | NP_060962.2 | 6 to 9 |
| PBXIP1 | 57326 | NM_020524.2 (SEQ ID NO: 161) | NP_065385.2 | 6, 7 |
| PLEK2 | 26499 | NM_016445.1 (SEQ ID NO: 162) | NP_057529.1 | 5 |
| PLK1 | 5347 | NM_005030.3 (SEQ ID NO: 163) | NP_005021.2 | 6, 8, 9 |
| PLK4 | 10733 | NM_014264.2 (SEQ ID NO: 164) | NP_055079.2 | 7, 9 |
| POLD1 | 5424 | NM_002691.1 (SEQ ID NO: 165) | NP_002682.1 | 5 |
| POLE | 5426 | NM_006231.2 (SEQ ID NO: 166) | NP_006222.2 | 5 |
| POLE2 | 5427 | NM_002692.2 (SEQ ID NO: 167) | NP_002683.2 | 5, 6, 8, 9 |
| POSTN | 10631 | NM_006475.1 (SEQ ID NO: 168) | NP_006466.1 | 7, 8 |
| PRC1 | 9055 | NM_199413.1 (SEQ ID NO: 169) | NP_955445.1 (isoform 2) | 5 to 9 |
| | | NM_003981.2 | NP_003972.1 (isoform 1) | |
| | | NM_199414.1 | NP_955446.1 (isoform 3) | |
| PRIM1 | 5557 | NM_000946.2 (SEQ ID NO: 170) | NP_000937.1 | 5 |
| PRKG2 | 5593 | NM_006259.1 (SEQ ID NO: 171) | NP_006250.1 | 7 |
| PSAT1 | 29968 | NM_058179.2 (SEQ ID NO: 172) | NP_478059.1 (isoform 1) | 6, 7 |
| | | NM_021154.3 | NP_066977.1 (isoform 2) | |
| PTTG1 | 9232 | NM_004219.2 (SEQ ID NO: 173) | NP_004210.1 | 5, 6, 8, 9 |
| RACGAP1 | 29127 | NM_013277.2 (SEQ ID NO: 174) | NP_037409.2 | 6, 8, 9 |
| RAD51 | 5888 | NM_133487.1 | NP_597994.1 (isoform 2) | 5 to 9 |
| | | NM_002875.2 (SEQ ID NO: 175) | NP_002866.2 (isoform 1) | |
| RAD51AP1 | 10635 | NM_006479.2 (SEQ ID NO: 176) | NP_006470.1 | 6, 7 |
| RBL1 | 5933 | NM_002895.2 (SEQ ID NO: 177) | NP_002886.2 | 5 |
| | | NM_183404.1 | NP_899662.1 | |
| RCC1 | 1104 | NM_001269.2 (SEQ ID NO: 178) | NP_001260.1 | 6, 8, 9 |
| RFC4 | 5984 | NM_002916.3 (SEQ ID NO: 179) | NP_002907.1 | 5, 6, 8, 9 |
| | | NM_181573.1 | NP_853551.1 | |
| RPL22 | 6146 | NM_000983.3 (SEQ ID NO: 180) | NP_000974.1 (proprotein) | 5, 6 |
| RRM1 | 6240 | NM_001033.2 (SEQ ID NO: 181) | NP_001024.1 | 5, 6 |
| RRM2 | 6241 | NM_001034.1 (SEQ ID NO: 182) | NP_001025.1 | 5 to 9 |
| SEMA3C | 10512 | NM_006379.2 (SEQ ID NO: 183) | NP_006370.1 | 5, 8 |
| SHCBP1 | 79801 | NM_024745.2 (SEQ ID NO: 184) | NP_079021.2 | 6 to 9 |
| SKP2 | 6502 | NM_032637.2 | NP_116026.1 (isoform 2) | 8, 9 |
| | | NM_005983.2 (SEQ ID NO: 185) | NP_005974.2 (isoform 1) | |
| SMC2 | 10592 | NM_006444.1 (SEQ ID NO: 186) | NP_006435.1 | 5, 6, 8, 9 |
| (SMC2L1) | | | | |
| SMC4 | 10051 | NM_001002799.1 | NP_001002799.1 (isoform b) | 5, 6, 8, 9 |
| (SMC4L1) | | NM_001002800.1 | NP_001002800.1 (isoform a) | |
| | | NM_005496.3 (SEQ ID NO: 187) | NP_005487.3 (isoform a) | |
| SORL1 | 6653 | NM_003105.3 (SEQ ID NO: 188) | NP_003096.1 (preproprotein) | 5, 6, 8, 9 |
| SPAG5 | 10615 | NM_006461.3 (SEQ ID NO: 189) | NP_006452.3 | 6 to 9 |
| SPBC25 | 57405 | NM_020675.3 (SEQ ID NO: 190) | NP_065726.1 | 6, 8, 9 |
| STEAP1 | 26871 | NM_012449.2 (SEQ ID NO: 191) | NP_036581.1 | 8, 9 |
| STMN1 | 3925 | NM_203399.1 | NP_981944.1 | 5, 6, 8, 9 |
| | | NM_005563.3 | NP_005554.1 | |
| | | NM_203401.1 (SEQ ID NO: 192) | NP_981946.1 | |
| SYNPO | 11346 | NM_007286.3 (SEQ ID NO: 193) | NP_009217.3 | 6, 8, 9 |
| TACC3 | 10460 | NM_006342.1 (SEQ ID NO: 194) | NP_006333.1 | 5 to 9 |
| TGFBR1 | 7046 | NM_004612.2 (SEQ ID NO: 195) | NP_004603.1 (precursor) | 7 |
| TIMELESS | 8914 | NM_003920.2 (SEQ ID NO: 196) | NP_003911.1 | 5, 6, 8, 9 |
| TK1 | 7083 | NM_003258.1 (SEQ ID NO: 197) | NP_003249.1 | 5, 6, 8, 9 |
| TLE4 | 7091 | NM_007005.3 (SEQ ID NO: 198) | NP_008936.2 | 6, 8 |
| TOP2A | 7153 | NM_001067.2 (SEQ ID NO: 199) | NP_001058.2 | 5 to 9 |
| TOPBP1 | 11073 | NM_007027.2 (SEQ ID NO: 200) | NP_008958.1 | 5, 9 |
| TPX2 | 22974 | NM_012112.4 (SEQ ID NO: 201) | NP_036244.2 | 6, 8, 9 |
| TRIB3 | 57761 | NM_021158.3 (SEQ ID NO: 202) | NP_066981.2 | 6, 8, 9 |
| TRIP13 | 9319 | NM_004237.2 (SEQ ID NO: 203) | NP_004228.1 | 5, 6, 8, 9 |
| TROAP | 10024 | NM_005480.2 (SEQ ID NO: 204) | NP_005471.2 | 5, 8, 9 |
| TTK | 7272 | NM_003318.3 (SEQ ID NO: 205) | NP_003309.2 | 5 to 9 |
| TXNIP | 10628 | NM_006472.1 (SEQ ID NO: 206) | NP_006463.2 | 6 to 9 |
| UBE2C | 11065 | NM_181802.1 (SEQ ID NO: 207) | NP_861518.1 (isoform 4) | 6, 8, 9 |
| | | NM_181799.1 | NP_861515.1 (isoform 2) | |
| | | NM_007019.2 | NP_008950.1 (isoform 1) | |
| | | NM_181800.1 | NP_861516.1 (isoform 3) | |
| | | NM_181803.1 | NP_861519.1 (isoform 5) | |
| | | NM_181801.1 | NP_861517.1 (isoform 4) | |
| WDHD1 | 11169 | NM_001008396.1 | NP_001008397.1 (isoform 2) | 7 |
| | | NM_007086.2 (SEQ ID NO: 208) | NP_009017.1 (isoform 1) | |
| WHSC1 | 7468 | NM_133330.1 | NP_579877.1 (isoform 1) | 6, 8, 9 |
| | | NM_133331.1 | NP_579878.1 (isoform 1) | |
| | | NM_133335.1 | NP_579890.1 (isoform 1) | |
| | | NM_007331.1 | NP_015627.1 (isoform 4) | |
| | | NM_133334.1 (SEQ ID NO: 209) | NP_579889.1 (isoform 3) | |
| | | NM_133336.1 | NP_579891.1 (isoform 5) | |
| WIZ | 58525 | XM_372716.5 (SEQ ID NO: 210) | XP_372716.5 (isoform 1) | 7 |
| ZBTB10 | 65986 | NM_023929.2 (SEQ ID NO: 211) | NP_076418.2 | 7 |
| ZWILCH | 55055 | NM_017975.2 (SEQ ID NO: 212) | NP_060445.2 | 8, 9 |

The expression level of each of the target molecules of Table 2 significantly changes in cells when the cells overexpress the Brd4 gene, which gene encodes the mRNA sequence of Accession No. NM_{—}058243 (SEQ ID NO: 3) or NM_{—}014299 (SEQ ID NO: 4) and encodes the amino acid sequence of Accession No. NP_{—}490597.1 (SEQ ID NO: 5) or NP_{—}055114.1 (SEQ ID NO: 6), which sequences are available from the GenBank database of the NCBI website. Ectopic expression of the Brd4 gene in the highly metatstatic mouse mammay tumor cell line Mvt-1 reduces cell invasiveness as well as the ability of the cells to form extensions in a three-dimensional culture. Also, ectopic expression of Brd4 in Mvt-1 reduces tumor growth and pulmonary surface metastsis following subcutaneous implantation of cells into FVB/NJ mice. Therefore, the expression levels of the target molecules of Table 2 are characteristic of a tumor or a cancer in a subject, e.g., are predictive of whether a subject afflicted with cancer, e.g., breast cancer, will survive, as further described herein.

In a preferred embodiment of the invention, the array comprises a set of addressable elements, such that the set comprises an addressable element specific for each of the target molecules of Table 2. In this regard, all of the target molecules of Table 2 are detected by the array. Alternatively or additionally, the set of addressable elements can consist essentially of addressable elements specific for cancer-related target molecules, as described herein, such that cancer-related target molecules are predominantly detected by the array. For example, the set of addressable elements can consist essentially of the addressable elements that are specific for the target molecules of Table 2, in combination with one or more addressable elements not listed in Table 2, e.g., a cancer-related target molecule (e.g., any of the target molecules listed in any of Table 1). Alternatively, the set can consist essentially of the addressable elements specific for the target molecules of Table 2.

The target molecules of Group 5 are target molecules of Table 2 which exhibit the same expression patterns (e.g., are either upregulated or downregulated in the same manner) in patients of the GSE1456 breast cancer cohort (Pawitan et al., Breast Cancer Res. 7: R953-R964 (2005)). Therefore, the expression levels of the target molecules of Group 5 are characteristic of a tumor or a cancer in a subject, e.g., are predictive of whether a subject afflicted with cancer, e.g., breast cancer, will survive, as described herein, especially if the tumor or cancer of the subject is similar to the tumor or cancer of the patients of the GSE1456 breast cancer cohort.

The target molecules of Group 6 are target molecules of Table 2 which exhibit the same expression patterns (e.g., are either upregulated or downregulated in the same manner) in patients of the GSE2034 breast cancer cohort (Wang et al., Lancet 365: 671-679 (2005)). Therefore, the expression levels of the target molecules of Group 6 are characteristic of a tumor or a cancer in a subject, e.g., are predictive of whether a subject afflicted with cancer, e.g., breast cancer, will survive, as described herein, especially if the tumor or cancer of the subject is similar to the tumor or cancer of the patients of the GSE2034 breast cancer cohort.

The target molecules of Group 7 are target molecules of Table 2 which exhibit the same expression patterns (e.g., are either upregulated or downregulated in the same manner) in patients of the GSE3494 breast cancer cohort (Miller et al., Proc. Natl. Acad. Sci. U.S.A. 102: 13550-13555 (2005)). Therefore, the expression levels of the target molecules of Group 7 are characteristic of a tumor or a cancer in a subject, e.g., are predictive of whether a subject afflicted with cancer, e.g., breast cancer, will survive, as described herein, especially if the tumor or cancer of the subject is similar to the tumor or cancer of the patients of the GSE3494 breast cancer cohort.

The target molecules of Group 8 are target molecules of Table 2 which exhibit the same expression patterns (e.g., are either upregulated or downregulated in the same manner) in patients of the GSE4922 breast cancer cohort (Ivashina et al., Cancer Res. 66: 10292-10301 (2006)). Therefore, the expression levels of the target molecules of Group 8 are characteristic of a tumor or a cancer in a subject, e.g., are predictive of whether a subject afflicted with cancer, e.g., breast cancer, will survive, as described herein, especially if the tumor or cancer of the subject is similar to the tumor or cancer of the patients of the GSE4922 breast cancer cohort.

The target molecules of Group 9 are target molecules of Table 1 which exhibit the same expression patterns (e.g., are either upregulated or downregulated in the same manner) in patients of the Rosetta breast cancer cohort (van't Veer et al., Nature 415: 530-536 (2002)). Therefore, the expression levels of the target molecules of Group 9 are characteristic of a tumor or a cancer in a subject, e.g., are predictive of whether a subject afflicted with cancer, e.g., breast cancer, will survive, as described herein, especially if the tumor or cancer of the subject is similar to the tumor or cancer of the patients of the Rosetta breast cancer cohort.

In one embodiment of the invention, the array comprises a set of addressable elements specific for the target molecules listed in Group 5, Group 6, Group 7, Group 8, Group 9, or any combination thereof (e.g., Groups 5-9, Groups 5-8, Groups 5-7, Groups 5 and 6, Groups 6-9, Groups 6-8, Groups 6 and 7, Groups 7-9, Groups 7 and 8, and Groups 8 and 9.)

In a preferred embodiment of the invention, the array comprises a set of addressable elements, such that the set comprises an addressable element specific for each of the target molecules of the Group(s). In this regard, all of the target molecules of the Group(s) are detected by the array. Alternatively or additionally, the set of addressable elements can consist essentially of addressable elements specific for cancer-related target molecules, as described herein, such that cancer-related target molecules are predominantly detected by the array. For example, the set of addressable elements can consist essentially of the addressable elements that are specific for the target molecules of the Group(s), in combination with one or more addressable elements not listed in the Group(s), e.g., a cancer-related target molecule (e.g., any of the target molecules listed in any of the other Group(s), Table 1, or a combination thereof). Alternatively, the set can consist essentially of the addressable elements specific for the target molecules of the Group(s).

The addressable elements of the array may be specific for target molecules other than the ones listed in Tables 1 and 2. For example, the addressable elements of the array may be specific for other target molecules no listed in Table 1 or 2. By “cancer-related target molecule” as used herein is meant any molecule, e.g., DNA, RNA, protein, for which the expression level is significantly changed in a cancer cell as compared to a normal, non-cancerous cell. For example, the array can advantageously comprise an addressable element that binds to one of the cancer-related target molecules p53, Src, Ras, or a combination thereof.

In a preferred embodiment of the invention, when the array of the invention is specific for 5 or more of the target molecules listed in Table 3, the array is specific for at least one target molecule listed in Table 1 and/or 2 and that is not listed in Table 3.

TABLE 3

| | Entrez | | |
|---|---|---|---|
| | Gene | GenBank Accession No. | |
| Target Molecule | ID No. | Nucleotide | Amino acid |
| TSPYL5 (AL080059) | 85453 | NM_033512.2 (SEQ ID NO: 213) | NP_277047.2 |
| FLT1 | 2321 | NM_002019.2 (SEQ ID NO: 214) | NP_002010.1 |
| MMP9 | 4318 | NM_004994.2 (SEQ ID NO: 215) | NP_004985.2 |
| C16orf61 (DC13) | 56942 | NM_020188.2 (SEQ ID NO: 216) | NP_064573.1 |
| EXT1 | 2131 | NM_000127.2 (SEQ ID NO: 217) | NP_000118.2 |
| DIAPH3 (AL137718) | 81624 | NM_030932.2 (SEQ ID NO: 218) | NP_112194.2 |
| CDC42BPA (PK428) | 8476 | NM_014826.3 (SEQ ID NO: 219) | NP_055641.3 |
| | | NM_003607.2 (SEQ ID NO: 220) | NP_003598.2 |
| NDC80 (HEC) | 10403 | NM_006101.1 (SEQ ID NO: 221) | NP_006092.1 |
| ECT2 | 1894 | NM_018098.4 (SEQ ID NO: 222) | NP_060568.3 |
| GMPS | 8833 | NM_003875.2 (SEQ ID NO: 223) | NP_003866.1 |
| UCHL5 (UCH37) | 51377 | NM_015984.1 (SEQ ID NO: 224) | NP_057068.1 |
| EXOC7 (KIAA1067) | 23265 | NM_015219.2 (SEQ ID NO: 225) | NP_056034.2 |
| | | NM_001013839.1 (SEQ ID NO: 226) | NP_001013861.1 |
| GNAZ | 2781 | NM_002073.2 (SEQ ID NO: 227) | NP_002064.1 |
| SERF1A | 8293 | NM_021967.1 (SEQ ID NO: 228) | NP_068802.1 |
| OXCT1 | 5019 | NM_000436.2(SEQ ID NO: 229) | NP_000427.1 |
| ORC6L | 23594 | NM_014321.2 (SEQ ID NO: 230) | NP_055136.1 |
| DTL (L2DTL) | 51514 | NM_016448.1 (SEQ ID NO: 231) | NP_057532.1 |
| PRC1 | 9055 | NM_199413.1 (SEQ ID NO: 232) | NP_955445.1 |
| | | NM_003981.2 (SEQ ID NO: 233) | NP_003972.1 |
| | | NM_199414.1(SEQ ID NO: 234) | NP_955446.1 |
| AYTL2 (AF052162) | 79888 | NM_024830.3 (SEQ ID NO: 235) | NP_079106.3 |
| COL4A2 | 1284 | NM_001846.1 (SEQ ID NO: 236) | NP_001837.1 |
| MELK (KIAA0175) | 9833 | NM_014791.2 (SEQ ID NO: 237) | NP_055606.1 |
| RAB6B | 51560 | NM_016577.2 (SEQ ID NO: 238) | NP_057661.2 |
| DCK | 1633 | NM_000788.1 (SEQ ID NO: 239) | NP_000779.1 |
| CENPA | 1058 | NM_001809.2 (SEQ ID NO: 240) | NP_001800.1 |
| EGLN1 (SM20) | 54583 | NM_022051.1 (SEQ ID NO: 241) | NP_071334.1 |
| MCM6 | 4175 | NM_005915.4 (SEQ ID NO: 242) | NP_005906.2 |
| PALM2-AKAP2 | 445815 | NM_007203.3 (SEQ ID NO: 243) | NP_009134.1 |
| | | NM_147150.1(SEQ ID NO: 244) | NP_671492.1 |
| RFC4 | 5984 | NM_002916.3 (SEQ ID NO: 245) | NP_002907.1 |
| | | NM_181573.1 (SEQ ID NO: 246) | NP_853551.1 |
| SLC2A3 | 6515 | NM_006931.1 (SEQ ID NO: 247) | NP_008862.1 |
| MAP2K1IP1 (MP1) | 8649 | NM_021970.2 (SEQ ID NO: 248) | NP_068805.1 |
| C20orf46 (FLJ11190) | 55321 | NM_018354.1 (SEQ ID NO: 249) | NP_060824.1 |
| IGFBP5 | 3488 | NM_000599.2 (SEQ ID NO: 250) | NP_000590.1 |
| CCNE2 | 9134 | NM_057749.1 (SEQ ID NO: 251) | NP_477097.1 |
| | | NM_057735.1 (SEQ ID NO: 252) | NP_477083.1 |
| ESM1 | 11082 | NM_007036.3 (SEQ ID NO: 253) | NP_008967.1 |
| NMU | 10874 | NM_006681.1 (SEQ ID NO: 254) | |
| HRASLS (LOC57110) | 57110 | NM_020386.2 (SEQ ID NO: 255) | NP_065119.1 |
| PECI | 10455 | NM_006117.2 (SEQ ID NO: 256) | NP_006108.2 |
| | | NM_206836.1 (SEQ ID NO: 257) | NP_996667.1 |
| AP2B1 | 163 | NM_001030006.1 (SEQ ID NO: 258) | NP_001025177.1 |
| | | NM_001282.2 (SEQ ID NO: 259) | NP_001273.1 |
| MS4A7 (CFFM4) | 58475 | NM_021201.4 (SEQ ID NO: 260) | NP_067024.1 |
| | | NM_206938.1 (SEQ ID NO: 261) | NP_996821.1 |
| | | NM_206939.1 (SEQ ID NO: 262) | NP_996822.1 |
| | | NM_206940.1 (SEQ ID NO: 263) | NP_996823.1 |
| TGFB3 | 7043 | NM_003239.1 (SEQ ID NO: 264) | NP_003230.1 |
| STK32B (HSA250839) | 55351 | NM_018401.1 (SEQ ID NO: 265) | NP_060871.1 |
| GSTM3 | 2947 | NM_000849.3 (SEQ ID NO: 266) | NP_000840.2 |
| BBC3 | 27113 | NM_014417.2 (SEQ ID NO: 267) | NP_055232.1 |
| SCUBE2 (CEGP1) | 57758 | NM_020974.1 (SEQ ID NO: 268) | NP_066025.1 |
| WISP1 | 8840 | NM_003882.2 (SEQ ID NO: 269) | NP_003873.1 |
| | | NM_080838.1 (SEQ ID NO: 270) | NP_543028.1 |
| ALDH4A1 (ALDH4) | 8659 | NM_003748.2 (SEQ ID NO: 271) | NP_003739.2 |
| | | NM_170726.1 (SEQ ID NO: 272) | NP_733844.1 |
| EBF4 (KIAA1442) | 57593 | XM_044921.7 (SEQ ID NO: 273) | XP_044921.7 |
| FGF18 | 8817 | NM_003862.1 (SEQ ID NO: 274) | NP_003853.1 |
| Contig63649RC | | AW014921 (SEQ ID NO: 281) | |
| NUSAP1 (LOC51203) | 51203 | NM_016359.2 (SEQ ID NO: 275) | NP_057443.1 |
| | | NM_018454.5 (SEQ ID NO: 276) | NP_060924.4 |
| Contig46218RC | — | AI813331 (SEQ ID NO: 295) | |
| Contig38288RC | — | AI554061 (SEQ ID NO: 296) | |
| AA555029RC | — | SEQ ID NO: 1 of U.S. Pat. No. 7,171,311 | |
| Contig28552RC | — | AA992378 (SEQ ID NO: 283) | |
| Contig32185RC | — | AI377418 (SEQ ID NO: 297) | |
| Contig35251RC | — | AI283268 (SEQ ID NO: 287) | |
| Contig55725RC | — | AI992158 (SEQ ID NO: 288) | |
| Contig56457RC | — | AI741117 (SEQ ID NO: 289) | |
| GPR126 (DKFZP564D0462) | 57211 | NM_020455.4 (SEQ ID NO: 277) | NP_065188.4 |
| | | NM_198569.1 (SEQ ID NO: 278) | NP_940971.1 |
| | | NM_001032394.1 (SEQ ID NO: 279) | NP_001027566.1 |
| | | NM_001032395.1 (SEQ ID NO: 280) | NP_001027567.1 |
| Contig40831RC | — | AI224578 (SEQ ID NO: 290) | |
| Contig24252RC | — | AW024884 (SEQ ID NO: 282) | |
| Contig51464RC | — | AI817737 (SEQ ID NO: 291) | |
| Contig20217RC | — | AA834945 (SEQ ID NO: 284) | |
| Contig63102RC | — | AI583960 (SEQ ID NO: 292) | |
| Contig46223RC | — | AA528243 (SEQ ID NO: 285) | |
| Contig55377RC | — | AI918032 (SEQ ID NO: 293) | |
| Contig48328RC | — | AI694320 (SEQ ID NO: 294) | |
| Contig32125RC | — | AA404325 (SEQ ID NO: 286) | |

The array also can include one or more elements that serve as a control, standard, or reference molecule, such as a housekeeping gene (e.g., Porphobilinogen deaminase (PBGD), glyceraldehyde-3-phosphatase dehydrogenase (GAPDH), and RNA transferase) to assist in the normalization of expression levels or the determination of nucleic acid quality and binding characteristics, reagent quality and effectiveness, hybridization success, analysis thresholds and success, etc. These other common aspects of the arrays or the addressable elements, as well as methods for constructing and using arrays, including generating, labeling, and attaching suitable probes to the substrate, consistent with the invention are well-known in the art. Other aspects of the array are as previously described herein with respect to the methods of the invention.

It will be appreciated, however, that an array capable of detecting a vast number of target moleculess (e.g., mRNA or polypeptide targets), such as arrays designed for comprehensive expression profiling of a cell line (e.g., gene profiling) or the like, are not economical or convenient for use as a diagnostic tool or screen for any particular condition, e.g., cancer. Thus, to facilitate the convenient use of the array as a diagnostic tool or screen, for example, in conjunction with the methods described herein, the array preferably comprises a limited number of addressable elements and preferably comprises addressable elements specific only for cancer-related target molecules.

In this regard, the array desirably comprises less than 38,500 addressable elements. More desirably, the array comprises less than about 33,000 addressable elements or less than about 14,500 addressable elements. Further desirably, the array comprises less than about 8400 addressable elements, e.g., less than about 5000 addressable elements, less than 2500 addressable elements, e.g., 1000, 500, 100.

Also preferred is that the array comprises a number of addressable elements, such that the expression levels of multiple cancer-related target molecules are detected. In this regard, the array preferably detects the expression of at least 3 different target molecules, if not 10 or more target molecules, e.g., 50, 100, 250, 500, 1000 or more target molecules.

The addressable element can comprise a detectable label, such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles). The detectable label can be directly attached (either covalently or non-covalently) to the polynucleotide or polypeptide probe of the addressable element. Alternatively, the detectable label can be indirectly attached to the polynucleotide or polypeptide probe of the addressable element. For example, the detectable label can be attached via a linker.

With regard to the inventive arrays, the substrate can be any rigid or semi-rigid support to which polynucleotides or polypeptides can be covalently or non-covalently attached. Suitable substrates include membranes, filters, chips, slides, wafers, fibers, beads, gels, capillaries, plates, polymers, microparticles, and the like. Materials that are suitable for substrates include, for example, nylon, glass, ceramic, plastic, silica, aluminosilicates, borosilicates, metal oxides such as alumina and nickel oxide, various clays, nitrocellulose, and the like.

The polynucleotide or polypeptide probes of the addressable elements can be attached to the substrate in a pre-determined 1-, 2-, or 3-dimensional arrangement, such that the pattern of hybridization or binding to a probe is easily correlated with the expression of a particular target molecule. Because the probes are located at specified locations on or in the substrate, the hybridization or binding patterns and intensities thereof create a unique expression profile, which can be interpreted in terms of expression levels of particular target molecules and can be correlated with characteristics of the tumor or cancer, as further described herein.

Polynucleotide and polypeptide probes can be generated by any suitable method (see, e.g., Sambrook et al., Molecular Cloning: A Laboratory Manual, 2^{nd} Ed., Cold Spring Harbor Press, Cold Spring Harbor, N.Y., 1989). For example, polynucleotide probes that specifically bind to the mRNA transcripts of the target molecules described herein can be created using the target molecules themselves (or fragments thereof) by routine techniques (e.g., PCR or synthesis) based on the nucleotide sequence of the target molecule. As used herein, the term “fragment” means a contiguous part or portion of a polynucleotide sequence comprising about 10 or more nucleotides, preferably about 15 or more nucleotides, more preferably about 20 or more nucleotides (e.g., about 30 or more or even about 50 or more nucleotides).

Alternatively, the polynucleotide probe can be designed based on the sequence of the target molecule using probe design software, such as, for example, LightCycler® Probe Design Software 2.0 (Roche Applied Science, Indianapolis, Ind.).

The exact nature of the polynucleotide probe is not critical to the invention; any probe that will selectively bind the target molecule can be used. Typically, the polynucleotide probes will comprise 10 or more nucleotides (e.g., 20 or more, 50 or more, or 100 or more nucleotides). In order to confer sufficient specificity, it will have a sequence identity to a compliment of the target sequence (or corresponding fragment thereof) of about 90% or more, preferably about 95% or more (e.g., about 98% or more or about 99% or more) as determined, for example, using the well-known Basic Local Alignment Search Tool (BLAST) algorithm (available through the National Center for Biotechnology Information (NCBI) website).

Similarly, polypeptide probes that bind to the protein or polypeptide target molecules, or a fragment thereof, described herein can be created using the amino acid sequences of the target molecules using routine techniques. As used herein, the term fragment means a contiguous part or portion of any of a polypeptide sequence comprising about 5 or more amino acids, preferably about 10 or more amino acids, more preferably about 15 or more amino acids (e.g., about 20 or more amino acids or even about 30 or more or 50 or more amino acids). For example, antibodies to the protein or polypeptide target molecules can be generated in a mammal using routine techniques, which antibodies can be harvested to serve as probes for the target molecules. The exact nature of the probe is not critical to the invention; any probe that will selectively bind to the protein or polypeptide target molecule can be used. Preferred probes include antibodies and antibody fragments (e.g., F(ab)₂′ fragments, single chain antibody variable region fragment (ScFv) chains, and the like). Antibodies suitable for detecting the target molecules can be prepared by routine methods, and are commercially available. See, for instance, Harlow et al., Antibodies: A Laboratory Manual, Cold Spring Harbor Publishers, Cold Spring Harbor, N.Y., 1988.

The invention also provides a kit comprising a set of user instructions and (i) a set of polynucleotides, (ii) a set of polypeptides, or (iii) a combination thereof, wherein the set of polynucleotides is specific for the target molecules listed in any of Tables 1 and 2, Groups 1-13, or a combination thereof, wherein the set of polypeptides is specific for the target molecules listed in any of Tables 1 and 2, Groups 1-13, or a combination thereof

The polynucleotides and polypeptides of the kit which may be referred to hereinafter as “probes” are as previously described herein with respect to the polynucleotide probes and polypeptide probes of the array. Indeed, the polynucleotides and/or polypeptides of the kit can be provided in the form of an array. Alternatively, the probes of the kit can be provided unattached to any substrate, e.g., provided as a solution or a solid (e.g., a lyophilate) in one or more vials. The kit also can comprise probes specific for other cancer-related target molecules known in the art. However, to facilitate convenient use in a method of characterizing a tumor or a cancer in a subject, such as any of the methods described herein, the set of probes is preferably limited to a reasonable number. Thus, the kit preferably comprises less than about 38,500 probes, e.g., less than about 33,000 probes, less than about 14,500 probes, less than about 8400 probes, and less than about 5000 probes.

Also preferred is that the kit comprises a number of probes, such that the expression levels of multiple cancer-related target molecules are detected. In this regard, the kit preferably minimally detects the expression of at least 3 different target molecules, if not 10 or more target molecules, e.g., 50, 100, 250, 500, 1000 or more target molecules.

The polynucleotides and polypeptides of the kit can comprise a detectable label, such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles). In preferred embodiments of the invention, the detectable label is attached (either covalently or non-covalently) to the probes of the kit.

The kit also can comprise an appropriate buffer, suitable controls or standards as described elsewhere herein, and written or electronic instructions. Other aspects of the kit are as previously described with respect to the methods or the array of this invention.

The invention also provides methods of characterizing a tumor or cancer in a subject. The method comprises detecting the expression levels of a set of target molecules in the subject, wherein the set of target molecules comprises the target molecules listed in any of Tables 1 and 2 or Groups 1-13. Preferably, the set of target molecules consists essentially or consists of the target molecules of any of Tables 1 and 2, Groups 1-13, or a combination thereof

The inventive method of characterizing a tumor or cancer can include characterizing one, two, or any number of tumor or cancer characteristics. Preferably, the method characterizes the tumor or cancer in terms of one or more of metastatic capacity, tumor stage, tumor grade, nodal involvement, regional metastasis, distant metastasis, tumor size, and/or sex hormone receptor status.

The term “metastatic capacity” as used herein is synonymous with the term “metastatic potential” and refers to the chance that a tumor will become metastatic. The metastatic capacity of a tumor can range from high to low, e.g., from 100% to 0%. In this respect, the metastatic capacity of a tumor can be, for instance, 100%, 90%, 80%, 75%, 60%, 50%, 40%, 30%, 25%, 15%, 10%, 5%, 3%, 1%, or 0%. For example, a tumor having a metastatic capacity of 100% is a tumor having a 100% chance of becoming metastatic. Also, a tumor having a metastatic capacity of 50%, for example, is a tumor having a 50% chance of becoming metastatic. Further, a tumor with a metastatic capacity of 25%, for instance, is a tumor having a 25% chance of becoming metastatic.

“Tumor stage” as used herein refers to whether the cells of the tumor or cancer have remained localized (e.g., cells of the tumor or cancer have not metastasized from the primary tumor), have metastasized to only regional or surrounding tissues relative to the site of the primary tumor, or have metastasized to tissues that are distant from the site of the primary tumor.

“Tumor grade” as used herein refers to the degree of abnormality of cancer cells, a measure of differentiation, and/or the extent to which cancer cells are similar in appearance and function to healthy cells of the same tissue type. The degree of differentiation often relates to the clinical behavior of the particular tumor. Based on the microscopic appearance of cancer cells, pathologists commonly describe tumor grade by degrees of severity. Such terms are standard pathology terms, and are known and understood by one of ordinary skill in the art (see Crawford et al., Breast Cancer Research 8:R16; e-publication on Mar. 21, 2006)).

“Nodal involvement” as used herein refers to the presence of a tumor cell within a lymph node as detected by, for example, microscopic examination of a section of a lymph node.

“Regional metastasis” as used herein means the metastasis of a tumor cell to a region that is relatively close to the origin, i.e., the site of the primary tumor. For example, regional metastasis includes metastasis of a tumor cell to a regional lymph node that drains the primary tumor, i.e., that is connected to the primary tumor by way of the lymphatic system. Also, regional metastasis can be, for instance, the metastasis of a tumor cell to the liver in the case of a primary tumor that is in contact with the portal circulation. Further, regional metastasis can be, for example, metastasis to a mesenteric lymph node in the case of colon cancer. Furthermore, regional metastasis can be, for instance, metastasis to an axillary lymph node in the case of breast cancer.

The term “distant metastasis” as used herein refers to metastasis of a tumor cell to a region that is non-contiguous with the primary tumor (e.g., not connected to the primary tumor by way of the lymphatic or circulatory system). For instance, distant metastasis can be metastasis of a tumor cell to the brain in the case of breast cancer, a lung in the case of colon cancer, and an adrenal gland in the case of lung cancer.

“Sex hormone receptor status” as used herein means the status of whether a sex hormone receptor is expressed in the tumor cells or cancer cells. Sex hormone receptors are known in the art, including, for instance, the estrogen receptor, the testosterone receptor, and the progesterone receptor. Preferably, when characterizing certain cancers, such as breast cancer, the sex hormone receptor is the estrogen receptor or progesterone receptor.

As the metastatic capacity, tumor stage, tumor grade, nodal involvement, regional metastasis, distant metastasis, tumor size, and sex hormone receptor status are factors when considering whether a subject will survive from the cancer, the inventive method of characterizing a tumor or cancer in a subject desirably predicts whether the subject will survive from the cancer.

Further, as, for instance, the metastatic capacity, tumor stage, tumor grade, nodal involvement, regional metastasis, distant metastasis, tumor size, and sex hormone receptor status are factors considered when determining a treatment for a subject afflicted with a tumor or cancer, the inventive method of characterizing a tumor or cancer in a subject desirably determines a treatment for a subject afflicted with a tumor or a cancer.

The expression of target molecules can be detected or measured by any suitable method. For example, the expression of target molecules can be detected or measured on the basis of the expression levels of the mRNA or protein encoded by the target molecules. Suitable methods of detecting or measuring mRNA include, for example, Northern Blotting, reverse-transcription PCR (RT-PCR), and real-time RT-PCR. Such methods are described in Sambrook et al., Molecular Cloning: A Laboratory Manual, 2^{nd} Ed., Cold Spring Harbor Press, Cold Spring Harbor, N.Y., 1989. Of these methods, real-time RT-PCR is used. In real-time PCR, which is described in Bustin, J. Mol. Endocrinology 25: 169-193 (2000), PCRs are carried out in the presence of a labled (e.g., fluorogenic) oligonucleotide probe that hybridizes to the amplicons. The probes can be double-labeled, for example, with a reporter fluorochrome and a quencher fluorochrome. When the probe anneals to the complementary sequence of the amplicon during PCR, the Taq polymerase, which possesses 5′ nuclease activity, cleaves the probe such that the quencher fluorochrome is displaced from the reporter fluorochrome, thereby allowing the latter to emit fluorescence. The resulting increase in emission, which is directly proportional to the level of amplicons, is monitored by a spectrophotometer. The cycle of amplification at which a particular level of fluorescence is detected by the spectrophotometer is called the threshold cycle, C_{T}. It is this value that is used to compare levels of amplicons. Probes suitable for detecting mRNA levels of the target molecules described herein are commercially available and/or can be prepared by routine methods, such as methods discussed elsewhere herein.

Suitable methods of detecting protein levels in a sample include Western Blotting, radio-immunoassay, and Enzyme-Linked Immunosorbent Assay (ELISA). Such methods are described in Nakamura et al., Handbook of Experimental Immunology, 4^{th} ed., Vol. 1, Chapter 27, Blackwell Scientific Publ., Oxford, 1987. When detecting proteins in a sample using an immunoassay, the sample is typically contacted with antibodies or antibody fragments (e.g., F(ab)₂′ fragments, single chain antibody variable region fragment (ScFv) chains, and the like) that specifically bind the protein or polypeptide target molecule. Antibodies and other polypeptides suitable for detecting the target molecules in conjunction with immunoassays are commercially available and/or can be prepared by routine methods, such as methods discussed elsewhere herein (e.g., Harlow et al., Antibodies: A Laboratory Manual, Cold Spring Harbor Publishers, Cold Spring Harbor, N.Y., 1988).

The immune complexes formed upon incubating the sample with the antibody are subsequently detected by any suitable method. In general, the detection of immune complexes is well-known in the art and can be achieved through the application of numerous approaches. These methods are generally based upon the detection of a label or marker, such as any radioactive, fluorescent, biological or enzymatic tags or labels of standard use in the art. U.S. Patents concerning the use of such labels include U.S. Pat. Nos. 3,817,837, 3,850,752, 3,939,350, 3,996,345, 4,277,437, 4,275,149 and 4,366,241.

For example, the antibody used to form the immune complexes can, itself, be linked to a detectable label, thereby allowing the presence of or the amount of the primary immune complexes to be determined. Alternatively, the first added component that becomes bound within the primary immune complexes can be detected by means of a second binding ligand that has binding affinity for the first antibody. In these cases, the second binding ligand is, itself, often an antibody, which can be termed a “secondary” antibody. The primary immune complexes are contacted with the labeled, secondary binding ligand, or antibody, under conditions effective and for a period of time sufficient to allow the formation of secondary immune complexes. The secondary immune complexes are then washed to remove any non-specifically bound labeled secondary antibodies or ligands, and the remaining label in the secondary immune complexes is then detected.

Other methods include the detection of primary immune complexes by a two-step approach. A second binding ligand, such as an antibody, that has binding affinity for the first antibody can be used to form secondary immune complexes, as described above. After washing, the secondary immune complexes can be contacted with a third binding ligand or antibody that has binding affinity for the second antibody, again under conditions effective and for a period of time sufficient to allow the formation of immune complexes (tertiary immune complexes). The third ligand or antibody is linked to a detectable label, allowing detection of the tertiary immune complexes thus formed. A number of other assays are contemplated; however, the invention is not limited as to which method is used.

In a preferred embodiment of the inventive method, the expression levels are detected with one of the arrays or kits of the invention.

The inventive methods of characterizing a tumor or a cancer in a subject can be performed in vitro or in vivo. Preferably, the method is carried out in vitro.

Also, the invention provides use of a compound with anti-cancer activity for the preparation of a medicament to treat or prevent cancer in a subject for whom the expression levels of a set of target molecules have been determined, wherein the set of target molecules comprises the target molecules listed in any of Tables 1 and 2, Groups 1-13, or a combination thereof. Preferably, the set of target molecules consists essentially or consists of the target molecules of any of Tables 1 and 2, Groups 1-13, or a combination thereof. In a preferred embodiment of the inventive method, the expression levels are detected with any of the arrays or kits of the invention.

The anti-cancer activity can be any anti-cancer activity, including, but not limited to the reduction or inhibition of any of uncontrolled cell growth, loss of cell adhesion, altered cell morphology, foci formation, colony formation, in vivo tumor growth, and metastasis. Suitable methods for assaying for anti-cancer activity are known in the art (see, for example, Gong et al., Proc Natl Acad Sci USA, 101(44):15724-15729 (2004)—Epub 2004 Oct. 21).

The compound having anti-cancer activity can be any compound, including, but not limited to a small molecular weight compound, peptide, peptidomimetic, macromolecule, natural product, synthetic compound, and semi-synthetic compound. The compound can be a compound known to have anti-cancer activity, such as, for instance, asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

For purposes herein, the cancer can be any cancer. As used herein, the term “cancer” is meant any malignant growth or tumor caused by abnormal and uncontrolled cell division that may spread to other parts of the body through the lymphatic system or the blood stream. The cancer can be any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor. Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer.

The cancer can be an epithelial cancer. As used herein the term “epithelial cancer” refers to an invasive malignant tumor derived from epithelial tissue that can metastasize to other areas of the body, e.g., a carcinoma. Preferably, the epithelial cancer is breast cancer. Alternatively, the cancer can be a non-epithelial cancer, e.g., a sarcoma, leukemia, myeloma, lymphoma, neuroblastoma, glioma, or a cancer of muscle tissue or of the central nervous system (CNS).

The cancer can be a non-epithelial cancer. As used herein, the term “non-epithelial cancer” refers to an invasive malignant tumor derived from non-epithelial tissue that can metastasize to other areas of the body.

The cancer can be a metastatic cancer or a non-metastatic (e.g., localized) cancer. As used herein, the term “metastatic cancer” refers to a cancer in which cells of the cancer have metastasized, e.g., the cancer is characterized by metastasis of a cancer cells. The metastasis can be regional metastasis or distant metastasis, as described herein. Preferably, the cancer is a metastatic cancer.

As used herein, the term “subject” is meant any living organism. Preferably, the subject is a mammal. The term “mammal” as used herein refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is further preferred that the mammals are from the order Artiodactyla, including Bovines (cows) and Swines (pigs) or of the order Perssodactyla, including Equines (horses). It is further preferred that the mammals are of the order Primates, Ceboids, or Simoids (monkeys) or of the order Anthropoids (humans and apes). An especially preferred mammal is the human.

With respect to the inventive methods and uses, the set of target molecules for which the expression levels are detected can be from a sample obtained from the subject. The sample can be any suitable sample. The sample can be a liquid or fluid sample, such as a sample of body fluid (e.g., blood, plasma, interstitial fluid, bile, lymph, milk, semen, saliva, urine, mucous, etc.), or a solid sample, such as a hair or tissue sample (e.g., liver tissue or tumor tissue sample), which can be processed prior to use. A sample also may include a cell or cell line created under experimental conditions, which is not directly isolated from a subject or host, or a product produced in cell culture by normal, non-tumor, or transformed cells (e.g., via recombinant DNA technology).

As used herein, the term “detect” with respect to the expression of target molecules means to determine the presence or absence of detectable expression of a target molecule. Thus, detection encompasses, but is not limited to, measuring or quantifying the expression level of a target molecule by any method. Preferably, the method involves detecting or measuring the expression of the target molecule in such a way as to facilitate the comparison of expression levels between samples.

### Examples

The following examples further illustrate the invention but, of course, should not be construed as in any way limiting its scope.

### Example 1

This example demonstrates the microarray analysis of mouse Mvt-1 cell lines ectopically expressing Brd4.

Affymetrix microarrays are used to compare gene expression in four Mvt-1 clonal isolates ectopically expressing Brd4 (Mvt-1/Brd4) and three Mvt-1 clonal isolates ectopically expressing β-galactosidase (Mvt-1/β-galactosidase). Total RNA from the clonal isolates is extracted using TRIzol Reagent (Life Technologies, Inc.) according to the standard protocol. Total RNA samples are subjected to DNase I treatment, and sample quantity and quality determined as described above. Purified total RNA for each clonal isolate are then pooled to produce a uniform sample containing 8 μg.

Double stranded cDNA is synthesized from this preparation using the SuperScript Choice System for cDNA Synthesis (Invitrogen, Carlsbad, Calif.) according to the protocol for Affymetrix GeneChip Eukaryotic Target Preparation. The double stranded cDNA is purified using the GeneChip Sample Cleanup Module (Qiagen, Valencia, Calif.). Synthesis of biotin-labeled cRNA is obtained by in vitro transcription of the purified template cDNA using the Enzo BioArray High Yield RNA Transcript Labeling Kit (T7) (Enzo Life Sciences, Inc., Farmingdale, N.Y.). cRNAs are purified using the GeneChip Sample Cleanup Module (Qiagen). Hybridization cocktails from each fragmentation reaction are prepared according to the Affymetrix GeneChip protocol. The hybridization cocktail is applied to the Affymetrix GeneChip Mouse Genome 430 2.0 arrays, processed on the Affymetrix Fluidics Station 400, and analyzed on an Agilent GeneArray Scanner with Affymetrix Microarray Suite version 5.0.0.032 software. Normalization is performed using the BRB-Array Tools software (Yang et al., Clin. Exp. Metastasis 21: 719-735 (2004) and Yang et al., Clin. Exp. Metastasis 22: 593-603 (2005)).

CEL files are analyzed using the Affymetrix GeneChip Probe Level Data RMA option of BRB ArrayTools 3.5.0. Genes with <1.5 fold-change from the gene's median value in 50% of samples, or a log-ratio variation P>0.01 are eliminated from analyses. To identify a Brd4 expression signature, the Class Comparison tool of BRB ArrayTools is performed, using a two-sample t-test with random variance univariate test. P-values for significance are computed based on 10,000 random permutations, at a nominal significance level of each univariate test of 0.0001. A total of 2,577 probe sets pass these criteria.

Examples of probe sets significantly up regulated and down regulated according to these criteria are listed in Tables 4 and 5, respectively.

TABLE 4

| | Fold difference of | | | |
|---|---|---|---|---|
| | geom means | | | |
| | (Transfected/Control | | | |
| | cell lines) | Probe set | Gene symbol | Description |
| 1 | 125.0 | 1419663_at | Ogn | osteoglycin |
| 2 | 90.9 | 1423100_at | Fos | FBJ osteosarcoma oncogene |
| 3 | 62.5 | 1423606_at | Postn | periostin, osteoblast specific |
| | | | | factor |
| 4 | 58.8 | 1448735_at | Cp | ceruloplasmin |
| 5 | 58.8 | 1419662_at | Ogn | osteoglycin |
| 6 | 52.6 | 1416239_at | Ass1 | argininosuccinate synthetase 1 |
| 7 | 41.7 | 1424214_at | 9130213B05Rik | RIKEN cDNA 9130213B05 |
| | | | | gene |
| 8 | 37.0 | 1417494_a_at | Cp | ceruloplasmin |
| 9 | 35.7 | 1428891_at | 9130213B05Rik | RIKEN cDNA 9130213B05 |
| | | | | gene |
| 10 | 33.3 | 1455393_at | Cp | ceruloplasmin |
| 11 | 28.6 | 1423859_a_at | Ptgds | prostaglandin D2 synthase |
| | | | | (brain) |
| 12 | 27.8 | 1434465_x_at | Vldlr | very low density lipoprotein |
| | | | | receptor |
| 13 | 27.0 | 1460251_at | Fas | Fas (TNF receptor superfamily |
| | | | | member) |
| 14 | 26.3 | 1424041_s_at | C1s | complement component 1, s |
| | | | | subcomponent |
| 15 | 25.6 | 1417900_a_at | Vldlr | very low density lipoprotein |
| | | | | receptor |

TABLE 5

| | Fold difference of geom means | | | |
|---|---|---|---|---|
| | (Transfected/Control | Affymetrix | | |
| | cell lines) | Probe set | Gene symbol | Description |
| 1 | 0.385 | 1452717_at | Slc25a24 | solute carrier family 25 (mitochondrial |
| | | | | carrier, phosphate carrier), member 24 |
| 2 | 0.375 | 1429158_at | Fbxo28 | F-box protein 28 |
| 3 | 0.364 | 1416068_at | Kars | lysyl-tRNA synthetase |
| 4 | 0.356 | 1418905_at | Nubp1 | nucleotide binding protein 1 |
| 5 | 0.353 | 1420592_a_at | Anp32e | acidic (leucine-rich) nuclear phosphoprotein |
| | | | | 32 family, member E |
| 6 | 0.351 | 1431686_a_at | Gmfb | glia maturation factor, beta |
| 7 | 0.350 | 1425472_a_at | Lmna | lamin A |
| 8 | 0.348 | 1447934_at | 9630033F20Rik | RIKEN cDNA 9630033F20 gene |
| 9 | 0.347 | 1416014_at | Abce1 | ATP-binding cassette, sub-family E (OABP), |
| | | | | member 1 |
| 10 | 0.337 | 1417773_at | Nans | N-acetylneuraminic acid synthase (sialic acid |
| | | | | synthase) |
| 11 | 0.331 | 1435379_at | AK122209 | cDNA sequence AK122209 |
| 12 | 0.325 | 1454702_at | 4930503L19Rik | RIKEN cDNA 4930503L19 gene |
| 13 | 0.319 | 1450569_a_at | Rbm14 | RNA binding motif protein 14 |
| 14 | 0.319 | 1456566_x_at | Rbm14 | RNA binding motif protein 14 |
| 15 | 0.317 | 1416308_at | Ugdh | UDP-glucose dehydrogenase |

Gene ontological (GO) analysis is performed using BRB ArrayTools, and reveal that 149 classes of genes are modulated in response to ectopic expression of Brd4 at the nominal 0.005 level of the LS permutation test or KS permutation test. Examples of the 149 classes of genes are shown in Table 6.

TABLE 6

| | | | | | LS | KS |
|---|---|---|---|---|---|---|
| | GO | | | Number of | Permutation | Permutation |
| | category | GO Term | GO description | genes | P-value | P-value |
| 1 | 785 | Cellular Component | chromatin | 44 | 1.00E−05 | 0.00018 |
| 2 | 5694 | Cellular Component | chromosome | 96 | 1.00E−05 | 1.00E−05 |
| 3 | 5739 | Cellular Component | mitochondrion | 78 | 1.00E−05 | 1.00E−05 |
| 4 | 5783 | Cellular Component | endoplasmic reticulum | 49 | 1.00E−05 | 0.00062 |
| 5 | 5886 | Cellular Component | plasma membrane | 98 | 1.00E−05 | 0.00019 |
| 6 | 9986 | Cellular Component | cell surface | 15 | 1.00E−05 | 6.00E−04 |
| 7 | 15630 | Cellular Component | microtubule cytoskeleton | 58 | 1.00E−05 | 0.00162 |
| 8 | 5102 | Molecular Function | receptor binding | 50 | 1.00E−05 | 1.00E−05 |
| 9 | 5125 | Molecular Function | cytokine activity | 19 | 1.00E−05 | 1.00E−05 |
| 10 | 5215 | Molecular Function | transporter activity | 99 | 1.00E−05 | 1.00E−05 |
| 11 | 15267 | Molecular Function | channel or pore class transporter activity | 24 | 1.00E−05 | 0.00086 |
| 12 | 15288 | Molecular Function | porin activity | 14 | 1.00E−05 | 0.00123 |
| 13 | 30234 | Molecular Function | enzyme regulator activity | 80 | 1.00E−05 | 0.00078 |
| 14 | 6091 | Biological Process | generation of precursor metabolites and | 64 | 1.00E−05 | 1.00E−04 |
| | | | energy | | | |
| 15 | 6325 | Biological Process | establishment and/or maintenance of | 22 | 1.00E−05 | 0.00177 |
| | | | chromatin architecture | | | |
| 16 | 6412 | Biological Process | protein biosynthesis | 49 | 1.00E−05 | 1.00E−05 |
| 17 | 6468 | Biological Process | protein amino acid phosphorylation | 61 | 1.00E−05 | 5.00E−04 |
| 18 | 6512 | Biological Process | ubiquitin cycle | 69 | 1.00E−05 | 0.00412 |
| 19 | 6793 | Biological Process | phosphorus metabolism | 82 | 1.00E−05 | 0.00045 |

Examination of the complete list of gene classes reveals that ectopic expression of Brd4 in Mvt-1 cells modulates expression of genes involved in processes such as cellular proliferation, cell cycle progression and chromatin structure. Furthermore, it is apparent that, at least in this cell line, Brd4 also regulates a number of processes that are critical to metastasis (e.g. cytoskeletal remodeling, cell adhesion, extracellular matrix expression).

This example identified genes of which the expression levels change in response to ectopic expression of Brd4.

### Example 2

This example demonstrates that the Mvt-1/Brd4 signature predicts outcome in multiple breast cancer expression datasets.

A high confidence human transcriptional signature of BRD4 gene expression signature is generated by mapping the most significantly differentially regulated genes (P<10⁻⁷) from mouse array data to human Affymetrix and the Rosetta probe set annotations. Specifically, 638 probe sets, whose differential expression demonstrated P<10⁻⁷, are selected. A gene list representing the probes is developed and used to map to the probe sets of the human U133 Affymetrix GeneChip using the Batch Search function of NetAffx located on the Affymetrix website. A human signature of 971 probe sets representing more than 350 genes is identified and is shown in Table 7.

TABLE 7

| Probe Set ID | Gene Symbol | Gene Title |
|---|---|---|
| 201872_s_at; 201873_s_at | ABCE1 | ATP-binding cassette, sub-family E (OABP), member 1 |
| 201963_at; 207275_s_at; | ACSL1 | acyl-CoA synthetase long-chain family member 1 |
| 1552619_a_at; 222608_s_at | ANLN | anillin, actin binding protein (scraps homolog, Drosophila) |
| 208103_s_at; 221505_at | ANP32E | acidic (leucine-rich) nuclear phosphoprotein 32 family, |
| | | member E /// acidic (leucine-rich) nuclear phosphoprotein 32 family, |
| | | member E |
| 204492_at | ARHGAP11A | Rho GTPase activating protein 11A |
| 212738_at; 37577_at | ARHGAP19 | Rho GTPase activating protein 19 |
| 218115_at | ASF1B | ASF1 anti-silencing function 1 homolog B (S. cerevisiae) |
| 219918_s_at; 232238_at; 239002_at | ASPM | asp (abnormal spindle)-like, microcephaly associated (Drosophila) |
| 218782_s_at; 222740_at; 228401_at; | ATAD2 | ATPase family, AAA domain containing 2 |
| 235266_at | | |
| 1554420_at; 1554980_a_at; 202672_s_at | ATF3 | activating transcription factor 3 |
| 204092_s_at; 208079_s_at; 208080_at | AURKA | aurora kinase A |
| 209464_at; 239219_at; | AURKB | aurora kinase B |
| 214390_s_at; 214452_at; 225285_at; | BCAT1 | branched chain aminotransferase 1, cytosolic |
| 226517_at | | |
| 201169_s_at; 201170_s_at | BHLHB2 | basic helix-loop-helix domain containing, class B, 2 |
| 1555826_at; 202094_at; 202095_s_at; | BIRC5 | Baculoviral IAP repeat-containing 5 (□emaphori) |
| 210334_x_at | | |
| 205733_at | BLM | Bloom syndrome |
| 209590_at; 209591_s_at; 211259_s_at; | BMP7 | Bone morphogenetic protein 7 (osteogenic protein 1) |
| 211260_at | | |
| 204531_s_at; 211851_x_at; | BRCA1 | breast cancer 1, early onset |
| 212949_at | BRRN1 | barren homolog 1 (Drosophila) |
| 209642_at; 215508_at; 215509_s_at; | BUB1 | BUB1 budding uninhibited by benzimidazoles 1 homolog (yeast) |
| 216275_at; 216277_at; 233445_at | | |
| 203755_at | BUB1B | BUB1 budding uninhibited by benzimidazoles 1 homolog beta (yeast) |
| 209182_s_at; 209183_s_at; | C10orf10 | chromosome 10 open reading frame 10 |
| 225372_at; 225373_at | C10orf54 | chromosome 10 open reading frame 54 |
| 219099_at | C12orf5 | chromosome 12 open reading frame 5 |
| 219166_at | C14orf104 | chromosome 14 open reading frame 104 |
| 1557755_at; 1557756_a_at; 232635_at; | C14orf145 | chromosome 14 open reading frame 145 |
| 233859_at; 244033_at | | |
| 223474_at | C14orf4 | chromosome 14 open reading frame 4 |
| 1553644_at | C14orf49 | chromosome 14 open reading frame 49 |
| 218447_at | C16orf61 | chromosome 16 open reading frame 61 |
| 217640_x_at | C18orf24 | chromosome 18 open reading frame 24 |
| 226242_at; 240803_at | C1orf131 | chromosome 1 open reading frame 131 |
| 220011_at; 222946_s_at | C1orf135 | chromosome 1 open reading frame 135 |
| 1553697_at; 1553698_a_at; 1555145_at; | C1orf96 | chromosome 1 open reading frame 96 |
| 225904_at | | |
| 1555229_a_at; 208747_s_at; 233042_at; | C1S | complement component 1, s subcomponent |
| 224690_at; 224693_at | C20orf108 | chromosome 20 open reading frame 108 |
| 225890_at; 242453_at | C20orf72 | chromosome 20 open reading frame 72 |
| 219004_s_at; 228597_at; 229671_s_at | C21orf45 | chromosome 21 open reading frame 45 |
| 226464_at; 228079_at; 235853_at; | C3orf58 | chromosome 3 open reading frame 58 |
| 241050_at; | | |
| 218518_at; 241169_at | C5orf5 | chromosome 5 open reading frame 5 |
| 229953_x_at; 242006_at; 244401_at | C6orf152 | chromosome 6 open reading frame 152 |
| 227534_at | C9orf21 | chromosome 9 open reading frame 21 |
| 1564084_at; 202715_at | CAD | Carbamoyl-phosphate synthetase 2, aspartate transcarbamylase, and |
| | | dihydroorotase |
| 1552421_a_at | CALR3 | calreticulin 3 |
| 202763_at; 236729_at | CASP3 | caspase 3, apoptosis-related cysteine peptidase |
| 206607_at; 225231_at; 225234_at; | CBL | Cas-Br-M (murine) ecotropic retroviral transforming sequence |
| 229010_at; 243475_at | | |
| 203418_at; 213226_at | CCNA2 | cyclin A2 |
| 214710_s_at; 228729_at | CCNB1 | cyclin B1 |
| 1560161_at; 202705_at; 232764_at; | CCNB2 | Cyclin B2 |
| 232768_at | | |
| 205034_at; 211814_s_at; | CCNE2 | cyclin E2 |
| 1559936_at; 204826_at; 204827_s_at; | CCNF | Cyclin F |
| 241551_at | | |
| 214151_s_at; 214152_at; 221156_x_at; | CCPG1 | cell cycle progression 1 |
| 221511_x_at; 222156_x_at | | |
| 202870_s_at | CDC20 | CDC20 cell division cycle 20 homolog (S. cerevisiae) |
| 201853_s_at | CDC25B | cell division cycle 25B |
| 1570624_at; 205167_s_at; 216914_at; | CDC25C | Cell division cycle 25C |
| 217010_s_at | | |
| 204126_s_at | CDC45L | CDC45 cell division cycle 45-like (S. cerevisiae) |
| 203967_at; 203968_s_at | CDC6 | CDC6 cell division cycle 6 homolog (S. cerevisiae) |
| 204510_at | CDC7 | CDC7 cell division cycle 7 (S. cerevisiae) |
| 223381_at | CDCA1 | cell division cycle associated 1 |
| 1560968_at; 226661_at; 236957_at | CDCA2 | Cell division cycle associated 2 |
| 221436_s_at; 223307_at | CDCA3 | cell division cycle associated 3 /// cell division cycle associated 3 |
| 224753_at | CDCA5 | cell division cycle associated 5 |
| 224428_s_at; 230060_at | CDCA7 | cell division cycle associated 7 /// cell division cycle associated 7 |
| 221520_s_at | CDCA8 | cell division cycle associated 8 |
| 210240_s_at; 213586_at | CDKN2D | cyclin-dependent kinase inhibitor 2D (p19, inhibits CDK4) |
| 1555758_a_at; 209714_s_at | CDKN3 | cyclin-dependent kinase inhibitor 3 (CDK2-associated dual specificity |
| | | phosphatase) |
| 207230_at; 227526_at | CDON | Cdon homolog (mouse) |
| 204962_s_at; 210821_x_at | CENPA | centromere protein A, 17 kDa |
| 205046_at | CENPE | centromere protein E, 312 kDa |
| 207331_at; 207828_s_at; 209172_s_at | CENPF | centromere protein F, 350/400ka (mitosin) |
| 231772_x_at | CENPH | centromere protein H |
| 218827_s_at; 243315_at; 243490_at | CEP192 | centrosomal protein 192 kDa |
| 205393_s_at; 205394_at; 238075_at | CHEK1 | CHK1 checkpoint homolog (S. pombe) |
| 210416_s_at | CHEK2 | CHK2 checkpoint homolog (S. pombe) |
| 1562673_at; 205021_s_at; 205022_s_at; | CHES1 | Checkpoint suppressor 1 |
| 218031_s_at; 222494_at; 229237_s_at; | | |
| 241984_at; 243842_at; 244208_at | | |
| 204233_s_at | CHKA | choline kinase alpha |
| 204266_s_at | CHKA /// LOC650122 | choline kinase alpha /// similar to choline kinase alpha isoform a |
| 1556985_at; 221065_s_at | CHST8 | Carbohydrate (N-acetylgalactosamine 4-0) sulfotransferase 8 |
| 200810_s_at; 200811_at; 225191_at; | CIRBP | cold inducible RNA binding protein |
| 228519_x_at; 230142_s_at | | |
| 1554264_at; 218252_at | CKAP2 | cytoskeleton associated protein 2 |
| 204170_s_at | CKS2 | CDC28 protein kinase regulatory subunit 2 |
| 1553120_at; 219621_at | CLSPN | claspin homolog (Xenopus laevis) |
| 1561144_at; 201774_s_at | CNAP1 | Chromosome condensation-related SMC-associated protein 1 |
| 1558034_s_at; 204846_at; 214282_at; | CP | ceruloplasmin (ferroxidase) |
| 227253_at; | | |
| 1557295_a_at; 202551_s_at; | CRIM1 | Cysteine rich transmembrane BMP regulator 1 (chordin-like) |
| 202552_s_at; 228496_s_at; 233073_at; | | |
| 242803_at | | |
| 205927_s_at | CTSE | cathepsin E |
| 203302_at; 224115_at | DCK | deoxycytidine kinase |
| 201571_s_at; 201572_x_at; 210137_s_at | DCTD | dCMP deaminase |
| 209383_at | DDIT3 | DNA-damage-inducible transcript 3 |
| 202887_s_at | DDIT4 | DNA-damage-inducible transcript 4 |
| 208151_x_at; 208718_at; 208719_s_at; | DDX17 | DEAD (Asp-Glu-Ala-Asp) box polypeptide 17 /// DEAD (Asp-Glu-Ala- |
| 213998_s_at; 230180_at | | Asp) box polypeptide 17 |
| 1558473_at; 226980_at; 233115_at | DEPDC1B | DEP domain containing 1B |
| 202532_s_at; 202534_x_at; 48808_at | DHFR /// LOC643509 | dihydrofolate reductase /// similar to Dihydrofolate reductase |
| 202533_s_at | DHFR /// LOC643509 /// | dihydrofolate reductase /// similar to Dihydrofolate reductase /// similar to |
| | LOC653874 | Dihydrofolate reductase |
| 213632_at | DHODH | dihydroorotate dehydrogenase |
| 202802_at; 207831_x_at; 211558_s_at | DHPS | deoxyhypusine synthase |
| 1558340_at; 1558342_x_at; 214724_at | DIXDC1 | DIX domain containing 1 |
| 204687_at; 225809_at | DKFZP564O0823 | DKFZP564O0823 protein |
| 218726_at | DKFZp762E1312 | hypothetical protein DKFZp762E1312 |
| 1556820_a_at; 1556821_x_at; | DLEU2 | deleted in lymphocytic leukemia, 2 |
| 1563229_at; 1569600_at; 216870_x_at; | | |
| 239936_at; 242854_x_at | | |
| 215629_s_at | DLEU2 /// DLEU2L | deleted in lymphocytic leukemia, 2 /// deleted in lymphocytic leukemia 2- |
| | | like |
| 1564443_at | DLEU2 /// RFP2OS | deleted in lymphocytic leukemia, 2 /// ret finger protein 2 opposite strand |
| 203764_at | DLG7 | discs, large homolog 7 (Drosophila) |
| 213647_at | DNA2L | DNA2 DNA replication helicase 2-like (yeast) |
| 213088_s_at; 213092_x_at | DNAJC9 | DnaJ (Hsp40) homolog, subfamily C, member 9 |
| 201697_s_at; 227684_at | DNMT1 | DNA (cytosine-5-)-methyltransferase 1 |
| 224814_at; 238012_at; 241973_x_at | DPP7 | dipeptidyl-peptidase 7 |
| 218585_s_at; 222680_s_at | DTL | denticleless homolog (Drosophila) |
| 201041_s_at; 201044_x_at; 226578_s_at | DUSP1 | dual specificity phosphatase 1 |
| 219990_at | E2F8 | E2F transcription factor 8 |
| 219787_s_at; 234992_x_at; 237241_at | ECT2 | epithelial cell transforming sequence 2 oncogene |
| 209392_at; 210839_s_at | ENPP2 | ectonucleotide pyrophosphatase/phosphodiesterase 2 (autotaxin) |
| 202609_at; 238371_s_at; 238372_s_at | EPS8 | epidermal growth factor receptor pathway substrate 8 |
| 1564473_at; 235178_x_at; 235588_at; | ESCO2 | Establishment of cohesion 1 homolog 2 (S. cerevisiae) |
| 241252_at | | |
| 204817_at; 38158_at | ESPL1 | extra spindle poles like 1 (S. cerevisiae) |
| 1554576_a_at; 211603_s_at; | ETV4 | ets variant gene 4 (E1A enhancer binding protein, E1AF) |
| 203348_s_at; 203349_s_at; 216375_s_at; | ETV5 | ets variant gene 5 (ets-related molecule) |
| 230102_at | | |
| 204774_at | EVI2A | ecotropic viral integration site 2A |
| 204603_at | EXO1 | exonuclease 1 |
| 209692_at; 243652_at | EYA2 | eyes absent homolog 2 (Drosophila) |
| 203358_s_at; 215006_at | EZH2 | enhancer of zeste homolog 2 (Drosophila) |
| 218248_at; 229196_at; 239368_at | FAM111A | family with sequence similarity 111, member A |
| 218602_s_at; 222685_at; 233655_s_at | FAM29A | family with sequence similarity 29, member A |
| 225684_at; 225686_at | FAM33A | family with sequence similarity 33, member A |
| 228069_at; 234944_s_at; 234945_at | FAM54A | family with sequence similarity 54, member A |
| 221591_s_at | FAM64A | family with sequence similarity 64, member A |
| 224871_at | FAM79A | family with sequence similarity 79, member A |
| 225687_at | FAM83D | family with sequence similarity 83, member D |
| 1568889_at; 1568891_x_at; 223545_at; | FANCD2 | Fanconi anemia, complementation group D2 |
| 242560_at | | |
| 204780_s_at; 204781_s_at; 215719_x_at; | FAS | Fas (TNF receptor superfamily, member 6) |
| 216252_x_at; 233820_at; 237522_at | | |
| 1554795_a_at; 1555480_a_at; | FBLIM1 | filamin binding LIM protein 1 |
| 1555483_x_at; 225258_at | | |
| 1555971_s_at; 1555972_s_at; 202271_at; | FBXO28 | F-box protein 28 |
| 202272_s_at | | |
| 218875_s_at; 234863_x_at | FBXO5 | F-box protein 5 |
| 204767_s_at; 204768_s_at | FEN1 | flap structure-specific endonuclease 1 |
| 1552921_a_at; 222843_at | FIGNL1 | fidgetin-like 1 |
| 222267_at; 235158_at | FLJ14803 | hypothetical protein FLJ14803 |
| 219544_at; 234745_at; 234757_at; | FLJ22624 | FLJ22624 protein |
| 236560_at | | |
| 228281_at | FLJ25416 | hypothetical protein FLJ25416 |
| 209189_at | FOS | v-fos FBJ murine osteosarcoma viral oncogene homolog |
| 202768_at | FOSB | FBJ murine osteosarcoma viral oncogene homolog B |
| 205409_at; 218880_at; 218881_s_at; | FOSL2 | FOS-like antigen 2 |
| 225262_at; 241824_at | | |
| 1553613_s_at | FOXC1 | forkhead box C1 |
| 202580_x_at | FOXM1 | forkhead box M1 |
| 1558996_at; 1560353_at; 1561166_a_at; | FOXP1 | forkhead box P1 |
| 1563157_at; 1570134_at; 215221_at; | | |
| 223287_s_at; 223936_s_at; 223937_at; | | |
| 224837_at; 224838_at; 230415_at; | | |
| 232096_x_at; 235444_at; 238712_at; | | |
| 240666_at; 241993_x_at; 243291_at; | | |
| 243878_at; 244535_at; 244845_at | | |
| 1555046_at; 1563223_a_at; 207590_s_at | FSHPRH1 | FSH primary response (LRPR1 homolog, rat) 1 |
| 217655_at; 218084_x_at; 224252_s_at | FXYD5 | FXYD domain containing ion transport regulator 5 |
| 210220_at | FZD2 | frizzled homolog 2 (Drosophila) |
| 203725_at | GADD45A | growth arrest and DNA-damage-inducible, alpha |
| 218313_s_at; 222587_s_at | GALNT7 | UDP-N-acetyl-alpha-D-galactosamine:polypeptide N- |
| | | acetylgalactosaminyltransferase 7 (GalNAc-T7) |
| 203178_at; 216733_s_at;; 231590_at; | GATM | glycine amidinotransferase (L-arginine:glycine amidinotransferase) |
| 231686_at; 235426_at; | | |
| 205164_at; 36475_at | GCAT | glycine C-acetyltransferase (2-amino-3-ketobutyrate coenzyme A ligase) |
| 220291_at | GDPD2 | glycerophosphodiester phosphodiesterase domain containing 2 |
| 219722_s_at | GDPD3 | glycerophosphodiester phosphodiesterase domain containing 3 |
| 205498_at; 241584_at | GHR | growth hormone receptor |
| 202543_s_at; 202544_at | GMFB | glia maturation factor, beta |
| 218350_s_at | GMNN | geminin, DNA replication inhibitor |
| 202615_at; 211426_x_at; 224861_at; | GNAQ | Guanine nucleotide binding protein (G protein), q polypeptide |
| 224862_at; 224863_at; 236238_at | | |
| 223487_x_at; 223488_s_at | GNB4 | guanine nucleotide binding protein (G protein), beta polypeptide 4 |
| 1553025_at; 213094_at; 233887_at | GPR126 | G protein-coupled receptor 126 |
| 205770_at; 225609_at; 237402_at | GSR | glutathione reductase |
| 202680_at | GTF2E2 | general transcription factor IIE, polypeptide 2, beta 34 kDa |
| 1555685_at; 206933_s_at; 221892_at; | H6PD | Hexose-6-phosphate dehydrogenase (glucose 1-dehydrogenase) |
| 226160_at | | |
| 220224_at | HAO1 | hydroxyacid oxidase (glycolate oxidase) 1 |
| 220085_at; 223556_at; 227350_at; | HELLS | helicase, lymphoid-specific |
| 234040_at; 242890_at | | |
| 1569380_a_at; 217168_s_at | HERPUD1 | Homocysteine-inducible, endoplasmic reticulum stress-inducible, ubiquitin- |
| | | like domain member 1 |
| 201944_at | HEXB | hexosaminidase B (beta polypeptide) |
| 213763_at; 219028_at; 224016_at; | HIPK2 | Homeodomain interacting protein kinase 2 |
| 224065_at; 224066_s_at; 225097_at; | | |
| 225115_at; 225116_at; 225368_at; | | |
| 240294_at | | |
| 209398_at | HIST1H1C | histone 1, H1c |
| 214455_at; 236193_at | HIST1H2BC | histone 1, H2bc |
| 221582_at; 231681_x_at | HIST3H2A | histone 3, H2a |
| 206074_s_at; 210457_x_at | HMGA1 | high mobility group AT-hook 1 |
| 208808_s_at; 236091_at; 243368_at | HMGB2 | high-mobility group box 2 |
| 1557029_at; 1562677_at; 207165_at; | HMMR | Hyaluronan-mediated motility receptor (RHAMM) |
| 209709_s_at | | |
| 206997_s_at; 214165_s_at; 225263_at | HS6ST1 | heparin sulfate 6-O-sulfotransferase 1 |
| 205543_at | HSPA4L | heat shock 70 kDa protein 4-like |
| 208937_s_at | ID1 | inhibitor of DNA binding 1, dominant negative helix-loop-helix protein |
| 204615_x_at; 208881_x_at; 233014_at; | IDI1 | isopentenyl-diphosphate delta isomerase 1 |
| 242065_x_at | | |
| 209929_s_at; 36004_at | IKBKG | inhibitor of kappa light polypeptide gene enhancer in B-cells, kinase gamma |
| 207072_at | IL18RAP | interleukin 18 receptor accessory protein |
| 206569_at | IL24 | interleukin 24 |
| 1566043_at; 1566044_at; 219769_at; | INCENP | Inner centromere protein antigens 135/155 kDa |
| 244862_at | | |
| 213447_at | IPW | imprinted in Prader-Willi syndrome |
| 229638_at | IRX3 | Iroquois related homeobox protein 3 |
| 201124_at; 201125_s_at; 214020_x_at; | ITGB5 | integrin, beta 5 |
| 214021_x_at | | |
| 205718_at; 227331_at; 236810_at | ITGB7 | integrin, beta 7 |
| 200079_s_at; 200840_at | KARS | lysyl-tRNA synthetase /// lysyl-tRNA synthetase |
| 210261_at | KCNK2 | potassium channel, subfamily K, member 2 |
| 1563608_a_at; 1569461_at; 1569462_x_at | KCNT1 | potassium channel, subfamily T, member 1 |
| 202503_s_at; 211713_x_at242486_at | KIAA0101 | KIAA0101 |
| 223254_s_at; 223255_at; 223256_at; | KIAA1333 | KIAA1333 |
| 223257_at; 223258_s_at | | |
| 1559060_a_at; 223997_at; 228250_at; | KIAA1961 | KIAA1961 gene |
| 228768_at; 243861_at | | |
| 204444_at | KIF11 | kinesin family member 11 |
| 221258_s_at | KIF18A | kinesin family member 18A /// kinesin family member 18A |
| 218755_at | KIF20A | kinesin family member 20A |
| 202183_s_at; 216969_s_at | KIF22 | kinesin family member 22 |
| 204709_s_at; 244427_at | KIF23 | kinesin family member 23 |
| 209408_at; 211519_s_at; 209680_s_at | KIF2C | kinesin family member 2C |
| 220266_s_at; 221841_s_at | KLF4 | Kruppel-like factor 4 (gut) |
| 206551_x_at; 221985_at; 221986_s_at; | KLHL24 | kelch-like 24 (Drosophila) |
| 226158_at; 242088_at | | |
| 206316_s_at | KNTC1 | kinetochore associated 1 |
| 204162_at | KNTC2 | kinetochore associated 2 |
| 201088_at; 211762_s_at | KPNA2 /// LOC643995 | karyopherin alpha 2 (RAG cohort 1, importin alpha 1) /// similar to Importin |
| | | alpha-2 subunit (Karyopherin alpha-2 subunit) (SRP1-alpha) (RAG cohort |
| | | protein 1) |
| 200821_at; 203041_s_at; 203042_at | LAMP2 | lysosomal-associated membrane protein 2 |
| 211768_at; 221581_s_at | LAT2 | linker for activation of T cells family, member 2 /// linker for activation of T |
| | | cells family, member 2 |
| 207409_at | LECT2 | leukocyte cell-derived chemotaxin 2 |
| 202726_at | LIG1 | ligase I, DNA, ATP-dependent |
| 219181_at | LIPG | lipase, endothelial |
| 1554600_s_at; 203411_s_at; | LMNA | lamin A/C |
| 212086_x_at; 212089_at; 214213_x_at; | | |
| 244225_x_at | | |
| 222039_at; 241569_at | LOC146909 | hypothetical protein LOC146909 |
| 235088_at; 238015_at | LOC201725 | hypothetical protein LOC201725 |
| 222336_at; 224990_at | LOC201895 | hypothetical protein LOC201895 |
| 226608_at; 242555_at | LOC388272 | similar to RIKEN cDNA 4921524J17 |
| 221195_at; 227268_at; 221194_s_at | LOC51136; /// DHX40P | PTD016 protein /// DEAH (Asp-Glu-Ala-His) box polypeptide 40 |
| | | pseudogene |
| 220341_s_at | LOC51149 | hypothetical LOC51149 |
| 1566902_at; 1566903_at; 1569933_at; | LRP8 | Low density lipoprotein receptor-related protein 8, apolipoprotein e receptor |
| 205282_at; 208433_s_at | | |
| 202736_s_at; 202737_s_at | LSM4 | LSM4 homolog, U6 small nuclear RNA associated (S. cerevisiae) |
| 205036_at; 241845_at | LSM6 | LSM6 homolog, U6 small nuclear RNA associated (S. cerevisiae) |
| 1566267_at; 202728_s_at; 202729_s_at; | LTBP1 | Latent transforming growth factor beta binding protein 1 |
| 240858_at | | |
| 219588_s_at | LUZP5 | leucine zipper protein 5 |
| 1554768_a_at; 203362_s_at | MAD2L1 | MAD2 mitotic arrest deficient-like 1 (yeast) |
| 224378_x_at; 227219_x_at; 232011_s_at | MAP1LC3A | microtubule-associated protein 1 light chain 3 alpha /// microtubule- |
| | | associated protein 1 light chain 3 alpha |
| 228468_at | MASTL | microtubule associated serine/threonine kinase-like |
| 202107_s_at | MCM2 | MCM2 minichromosome maintenance deficient 2, mitotin (S. cerevisiae) |
| 201555_at | MCM3 | MCM3 minichromosome maintenance deficient 3 (S. cerevisiae) |
| 212141_at; 212142_at; 222036_s_at; | MCM4 | MCM4 minichromosome maintenance deficient 4 (S. cerevisiae) |
| 222037_at | | |
| 201755_at; 216237_s_at | MCM5 | MCM5 minichromosome maintenance deficient 5, cell division cycle 46 |
| | | (S. cerevisiae) |
| 201930_at; 238977_at | MCM6 | MCM6 minichromosome maintenance deficient 6 (MIS5 homolog, |
| | | S. pombe) (S. cerevisiae) |
| 208795_s_at; 210983_s_at | MCM7 | MCM7 minichromosome maintenance deficient 7 (S. cerevisiae) |
| 204825_at | MELK | maternal embryonic leucine zipper kinase |
| 1562830_at; 1565898_at; 1565900_at; | METT5D1 | Methyltransferase 5 domain containing 1 |
| 1566278_at; 1567663_at; 1567664_at; | | |
| 238773_at; 242247_at; 243736_at | | |
| 237046_x_at | MGC34647 | hypothetical protein MGC34647 |
| 212020_s_at; 212021_s_at; 212022_s_at; | MKI67 | antigen identified by monoclonal antibody Ki-67 |
| 212023_s_at; | | |
| 206426_at; 206427_s_at | MLANA | melan-A |
| 218883_s_at; 229304_s_at; 229305_at | MLF1IP | MLF1 interacting protein |
| 238025_at | MLKL | mixed lineage kinase domain-like |
| 1556306_at; 223189_x_at; 223190_s_at; | MLL5 | Myeloid/lymphoid or mixed-lineage leukemia 5 (trithorax homolog, |
| 226100_at | | Drosophila) |
| 218211_s_at; 229150_at | MLPH | melanophilin |
| 205680_at | MMP10 | matrix metallopeptidase 10 (stromelysin 2) |
| 205828_at | MMP3 | matrix metallopeptidase 3 (stromelysin 1, progelatinase) |
| 205235_s_at | MPHOSPH1 | M-phase phosphoprotein 1 |
| 205429_s_at | MPP6 | membrane protein, palmitoylated 6 (MAGUK p55 subfamily member 6) |
| 205395_s_at; 211334_at; 242456_at | MRE11A | MRE11 meiotic recombination 11 homolog A (S. cerevisiae) |
| 1554126_at; 1554127_s_at; 1566481_at; | MSRB3 | methionine sulfoxide reductase B3 |
| 1566482_at; 225782_at; 225790_at; | | |
| 238583_at | | |
| 206800_at; 217070_at; 217071_s_at; | MTHFR | 5,10-methylenetetrahydrofolate reductase (NADPH) |
| 226929_at; 239035_at | | |
| 204101_at; 234596_at; 234600_at; | MTM1 | myotubularin 1 |
| 36920_at | | |
| 213422_s_at; 228576_s_at | MXRA8 | matrix-remodelling associated 8 |
| 205951_at | MYH1 | myosin, heavy polypeptide 1, skeletal muscle, adult |
| 220319_s_at; 223129_x_at; 223130_s_at; | MYLIP | myosin regulatory light chain interacting protein |
| 227707_at; 228097_at; 228098_s_at | | |
| 218189_s_at; 241923_x_at | NANS | N-acetylneuraminic acid synthase (sialic acid synthase) |
| 201969_at; 201970_s_at; 242918_at | NASP | nuclear autoantigenic sperm protein (histone-binding) |
| 209159_s_at | NDRG4 | NDRG family member 4 |
| 1566114_at; 1566115_at; 212445_s_at; | NEDD4L | Neural precursor cell expressed, developmentally down-regulated 4-like |
| 212448_at | | |
| 219502_at | NEIL3 | nei endonuclease VIII-like 3 (E. coli) |
| 204641_at; 211080_s_at | NEK2 | NIMA (never in mitosis gene a)-related kinase 2 |
| 1567013_at; 1567014_s_at; 1567015_at; | NFE2L2 | nuclear factor (erythroid-derived 2)-like 2 |
| 201146_at; 239240_at; 243113_at | | |
| 203574_at | NFIL3 | nuclear factor, interleukin 3 regulated |
| 203927_at | NFKBIE | nuclear factor of kappa light polypeptide gene enhancer in B-cells inhibitor, |
| | | epsilon |
| 201577_at; 226797_at | NME1 | non-metastatic cells 1, protein (NM23A) expressed in |
| 204501_at; 214321_at | NOV | nephroblastoma overexpressed gene |
| 213040_s_at; 217041_at | NPTXR | neuronal pentraxin receptor |
| 203814_s_at; 244855_at | NQO2 | NAD(P)H dehydrogenase, quinone 2 |
| 204589_at | NUAK1 | NUAK family, SNF1-like kinase, 1 |
| 203978_at | NUBP1 | nucleotide binding protein 1 (MinD homolog, E. coli) |
| 218768_at | NUP107 | nucleoporin 107 kDa |
| 1556432_at; 202184_s_at; 233420_at; | NUP133 | Nucleoporin 133 kDa |
| 233421_s_at; 236905_at | | |
| 212247_at; 222382_x_at | NUP205 | nucleoporin 205 kDa |
| 202188_at; 241758_at | NUP93 | nucleoporin 93 kDa |
| 1562163_at; 218039_at; 219978_s_at | NUSAP1 | Nucleolar and spindle associated protein 1 |
| 219100_at; 240824_at | OBFC1 | oligonucleotide/oligosaccharide-binding fold containing 1 |
| 218730_s_at; 222722_at | OGN | osteoglycin (osteoinductive factor, mimecan) |
| 219105_x_at | ORC6L | origin recognition complex, subunit 6 like (yeast) |
| 1558017_s_at; 204004_at; 204005_s_at; | PAWR | PRKC, apoptosis, WT1, regulator |
| 214090_at; 214237_x_at; 226223_at; | | |
| 226231_at; 229515_at | | |
| 219148_at | PBK | PDZ binding kinase |
| 207838_x_at; 212259_s_at; 214176_s_at; | PBXIP1 | pre-B-cell leukemia transcription factor interacting protein 1 |
| 214177_s_at | | |
| 219295_s_at | PCOLCE2 | procollagen C-endopeptidase enhancer 2 |
| 1563467_at; 218718_at; 222719_s_at | PDGFC | Platelet derived growth factor C |
| 205251_at; 208518_s_at; 242892_at | PER2 | period homolog 2 (Drosophila) |
| 207132_x_at; 210908_s_at | PFDN5 | prefoldin subunit 5 |
| 1558666_at; 210617_at | PHEX | Phosphate regulating endopeptidase homolog, X-linked (hypophosphatemia, |
| | | vitamin D resistant rickets) |
| 203335_at | PHYH | phytanoyl-CoA 2-hydroxylase |
| 205281_s_at; 215969_at | PIGA | phosphatidylinositol glycan, class A (paroxysmal nocturnal |
| | | hemoglobinuria) /// phosphatidylinositol glycan, class A (paroxysmal |
| | | nocturnal hemoglobinuria) |
| 209018_s_at | PINK1 | PTEN induced putative kinase 1 |
| 209019_s_at | PINK1 | PTEN induced putative kinase 1 |
| 218644_at | PLEK2 | pleckstrin 2 |
| 202240_at | PLK1 | polo-like kinase 1 (Drosophila) |
| 201429_s_at | PLK1 /// RPL37A | polo-like kinase 1 (Drosophila) /// ribosomal protein L37a |
| 204886_at; 204887_s_at; 211088_s_at | PLK4 | polo-like kinase 4 (Drosophila) |
| 209034_at | PNRC1 | proline-rich nuclear receptor coactivator 1 |
| 203422_at | POLD1 | polymerase (DNA directed), delta 1, catalytic subunit 125 kDa |
| 1560509_at; 1561940_at216026_s_at | POLE | Polymerase (DNA directed), epsilon |
| 205909_at | POLE2 | polymerase (DNA directed), epsilon 2 (p59 subunit) |
| 1555777_at; 1555778_a_at; 210809_s_at; | POSTN | periostin, osteoblast specific factor |
| 214981_at; 228481_at | | |
| 235113_at; 242154_x_at | PPIL5 | peptidylprolyl isomerase (cyclophilin)-like 5 |
| 218009_s_at | PRC1 | protein regulator of cytokinesis 1 |
| 205053_at | PRIM1 | primase, polypeptide 1, 49 kDa |
| 207505_at | PRKG2 | protein kinase, cGMP-dependent, type II |
| 203650_at; 234340_at; 234346_x_at | PROCR | protein C receptor, endothelial (EPCR) |
| 220892_s_at; 223062_s_at | PSAT1 | phosphoserine aminotransferase 1 |
| 211663_x_at; 211748_x_at; 212187_x_at | PTGDS | prostaglandin D2 synthase 21 kDa (brain) /// prostaglandin D2 synthase |
| | | 21 kDa (brain) |
| 206084_at; 210675_s_at | PTPRR | protein tyrosine phosphatase, receptor type, R |
| 203554_x_at | PTTG1 | pituitary tumor-transforming 1 |
| 210127_at; 221792_at; 225259_at | RAB6B | RAB6B, member RAS oncogene family |
| 222077_s_at | RACGAP1 | Rac GTPase activating protein 1 |
| 223417_at; 224200_s_at; 238670_at; | RAD18 | RAD18 homolog (S. cerevisiae) |
| 238748_at | | |
| 205023_at; 205024_s_at | RAD51 | RAD51 homolog (RecA homolog, E. coli) (S. cerevisiae) |
| 204146_at | RAD51AP1 | RAD51 associated protein 1 |
| 1553535_a_at; 212125_at; 212127_at | RANGAP1 | Ran GTPase activating protein 1 |
| 1555003_at; 1555004_a_at; 1559307_s_at | RBL1 | retinoblastoma-like 1 (p107) |
| 1555639_a_at; 204178_s_at | RBM14 | RNA binding motif protein 14 |
| 206499_s_at; 215747_s_at | RCC1 | regulator of chromosome condensation 1 |
| 204023_at | RFC4 | replication factor C (activator 1) 4, 37 kDa |
| 203209_at; 203210_s_at | RFC5 | replication factor C (activator 1) 5, 36.5 kDa |
| 1556662_at | RHOQ | Ras homolog gene family, member Q |
| 1556663_s_at; 1559582_at; 212117_at; | RHOQ | Ras homolog gene family, member Q |
| 212119_at; 212120_at; 214449_s_at; | | |
| 239258_at | | |
| 212122_at | RHOQ /// LOC284988 | ras homolog gene family, member Q /// hypothetical LOC284988 |
| 201756_at | RPA2 | replication protein A2, 32 kDa |
| 208768_x_at; 214042_s_at; 220960_x_at; | RPL22 | ribosomal protein L22 |
| 221726_at; 221775_x_at; 237940_s_at; | | |
| 237941_at | | |
| 201476_s_at; 201477_s_at | RRM1 | ribonucleotide reductase M1 polypeptide |
| 201890_at; 209773_s_at | RRM2 | ribonucleotide reductase M2 polypeptide |
| 231895_at | SASS6 | spindle assembly 6 homolog (C. elegans) |
| 1552256_a_at; 201819_at; 215834_x_at; | SCARB1 | scavenger receptor class B, member 1 |
| 215835_at; 232421_at; 233991_at; | | |
| 233994_at | | |
| 217855_x_at; 221972_s_at; 224472_x_at; | SDF4 | stromal cell derived factor 4 |
| 232032_x_at | | |
| 203070_at; 203071_at | SEMA3B | sema domain, immunoglobulin domain (Ig), short basic domain, secreted, |
| | | (semaphorin) 3B |
| 203788_s_at; 203789_s_at; 236947_at; | SEMA3C | sema domain, immunoglobulin domain (Ig), short basic domain, secreted, |
| 240815_at | | (semaphorin) 3C |
| 204614_at | SERPINB2 | serpin peptidase inhibitor, clade B (ovalbumin), member 2 |
| 223195_s_at; 223196_s_at; 1553869_at; | SESN2 | sestrin 2 |
| 235683_at; 235684_s_at; 243546_at | | |
| 220357_s_at; 230573_at | SGK2 | serum/glucocorticoid regulated kinase 2 |
| 1553690_at; 231938_at | SGOL1 | shugoshin-like 1 (S. pombe) |
| 230165_at; 235425_at | SGOL2 | shugoshin-like 2 (S. pombe) |
| 219493_at | SHCBP1 | SHC SH2-domain binding protein 1 |
| 203625_x_at; 203626_s_at; 210567_s_at | SKP2 | S-phase kinase-associated protein 2 (p45) |
| 209610_s_at; 209611_s_at; 212810_s_at; | SLC1A4 | solute carrier family 1 (glutamate/neutral amino acid transporter), member 4 |
| 212811_x_at; 235875_at; 244377_at | | |
| 1569121_at; 204342_at; 241229_at; | SLC25A24 | solute carrier family 25 (mitochondrial carrier; phosphate carrier), member |
| 244481_at | | 24 |
| 212907_at; 228181_at; 242716_at | SLC30A1 | Solute carrier family 30 (zinc transporter), member 1 |
| 225295_at; 226444_at; 238968_at | SLC39A10 | solute carrier family 39 (zinc transporter), member 10 |
| 1554332_a_at; 219911_s_at; 229239_x_at | SLCO4A1 | Solute carrier organic anion transporter family, member 4A1 |
| 204240_s_at; 213253_at | SMC2L1 | SMC2 structural maintenance of chromosomes 2-like 1 (yeast) |
| 201663_s_at; 201664_at;; 215623_x_at; | SMC4L1 | SMC4 structural maintenance of chromosomes 4-like 1 (yeast) |
| 237246_at | | |
| 1553148_a_at; 213292_s_at; 215366_at; | SNX13 | sorting nexin 13 |
| 215820_x_at | | |
| 203509_at; 230707_at | SORL1 | sortilin-related receptor, L(DLR class) A repeats-containing |
| 203145_at | SPAG5 | sperm associated antigen 5 |
| 235572_at | SPBC24 | spindle pole body component 24 homolog (S. cerevisiae) |
| 209891_at | SPBC25 | spindle pole body component 25 homolog (S. cerevisiae) |
| 218817_at; 222753_s_at | SPCS3 | signal peptidase complex subunit 3 homolog (S. cerevisiae) |
| 202400_s_at; 202401_s_at | SRF | serum response factor (c-fos serum response element-binding transcription |
| | | factor) |
| 205542_at | STEAP1 | six transmembrane epithelial antigen of the prostate 1 |
| 200783_s_at; 217714_x_at | STMN1 | stathmin 1/oncoprotein 18 |
| 224724_at; 233555_s_at | SULF2 | sulfatase 2 |
| 218619_s_at | SUV39H1 | suppressor of variegation 3-9 homolog 1 (Drosophila) |
| 1554572_a_at; 219262_at | SUV39H2 | suppressor of variegation 3-9 homolog 2 (Drosophila) |
| 202796_at; 235128_at; 235914_at | SYNPO | synaptopodin |
| 1569487_at; 218308_at | TACC3 | Transforming, acidic coiled-coil containing protein 3 |
| 233320_at | TCAM1 | testicular cell adhesion molecule 1 homolog (mouse) |
| 204043_at | TCN2 | transcobalamin II; macrocytic anemia |
| 206943_at; 224793_s_at; 236561_at; | TGFBR1 | transforming growth factor, beta receptor I (activin A receptor type II-like |
| 239605_x_at | | kinase, 53 kDa) |
| 206409_at; 213135_at; 231536_at | TIAM1 | T-cell lymphoma invasion and metastasis 1 |
| 203046_s_at; 215455_at | TIMELESS | timeless homolog (Drosophila) |
| 1554408_a_at; 202338_at; 243103_at | TK1 | thymidine kinase 1, soluble |
| 204872_at; 214688_at; 216997_x_at; | TLE4 | transducin-like enhancer of split 4 (E(sp1) homolog, Drosophila) |
| 233575_s_at; 235765_at | | |
| 218073_s_at; 234672_s_at | TMEM48 | transmembrane protein 48 |
| 203508_at | TNFRSF1B | tumor necrosis factor receptor superfamily, member 1B |
| 201812_s_at | TOMM7 /// LOC201725 | translocase of outer mitochondrial membrane 7 homolog (yeast) /// |
| | | hypothetical protein LOC201725 |
| 201291_s_at; 201292_at; 237469_at | TOP2A | topoisomerase (DNA) II alpha 170 kDa |
| 1561924_at; 202633_at | TOPBP1 | Topoisomerase (DNA) II binding protein 1 |
| 210052_s_at | TPX2 | TPX2, microtubule-associated, homolog (Xenopus laevis) |
| 1555788_a_at; 218145_at | TRIB3 | tribbles homolog 3 (Drosophila) |
| 233669_s_at | TRIM54 | tripartite motif-containing 54 |
| 227801_at; 235476_at | TRIM59 | tripartite motif-containing 59 |
| 204033_at | TRIP13 | thyroid hormone receptor interactor 13 |
| 1568596_a_at; 204649_at | TROAP | trophinin associated protein (tastin) |
| 204822_at | TTK | TTK protein kinase |
| 226181_at | TUBE1 | tubulin, epsilon 1 |
| 201008_s_at; 201009_s_at; 201010_s_at | TXNIP | thioredoxin interacting protein |
| 1558356_at; 223279_s_at; 236715_x_at; | UACA | uveal autoantigen with coiled-coil domains and ankyrin repeats |
| 238868_at | | |
| 1294_at; 203281_s_at | UBE1L | ubiquitin-activating enzyme E1-like |
| 202954_at | UBE2C | ubiquitin-conjugating enzyme E2C |
| 223229_at | UBE2T | ubiquitin-conjugating enzyme E2T (putative) |
| 203343_at | UGDH | UDP-glucose dehydrogenase |
| 225655_at | UHRF1 | ubiquitin-like, containing PHD and RING finger domains, 1 |
| 202706_s_at; 202707_at; 215165_x_at | UMPS | uridine monophosphate synthetase (orotate phosphoribosyl transferase and |
| | | orotidine-5′-decarboxylase) |
| 226899_at; 239136_at | UNC5B | unc-5 homolog B (C. elegans) |
| 202412_s_at; 202413_s_at; 244520_at | USP1 | ubiquitin specific peptidase 1 |
| 201099_at; 201100_s_at; 229573_at | USP9X | ubiquitin specific peptidase 9, X-linked |
| 209822_s_at | VLDLR | very low density lipoprotein receptor |
| 1553778_at | WBSCR27 | Williams Beuren syndrome chromosome region 27 |
| 204727_at; 204728_s_at; 216228_s_at | WDHD1 | WD repeat and HMG-box DNA binding protein 1 |
| 209592_s_at; 221744_at; 221745_at; | WDR68 | WD repeat domain 68 |
| 224730_at; 224748_at; 233782_at; | | |
| 236134_at; 240675_at | | |
| 1557780_at; 209052_s_at; 209053_s_at; | WHSC1 | Wolf-Hirschhorn syndrome candidate 1 |
| 209054_s_at; 222777_s_at; 222778_s_at; | | |
| 223472_at; 242311_x_at; 244140_at | | |
| 221783_at; 221784_at; 221785_at; | WIZ | widely-interspaced zinc finger motifs |
| 52005_at | | |
| 1552737_s_at;; 1554580_a_at; | WWP2 | WW domain containing E3 ubiquitin protein ligase 2 |
| 204022_at; 210200_at; 240384_at; | | |
| 241125_at; 243787_at | | |
| 1560386_at; 208775_at; 217577_at; | XPO1 | Exportin 1 (CRM1 homolog, yeast) |
| 217578_at | | |
| 218069_at | XTP3TPA | XTP3-transactivated protein A |
| 223179_at; 232077_s_at | YPEL3 | yippee-like 3 (Drosophila) |
| 219312_s_at; 222863_at; 233899_x_at; | ZBTB10 | zinc finger and BTB domain containing 10 |
| 235491_at; 235726_at; 242174_at | | |
| 1563502_at; 222730_s_at; 222731_at; | ZDHHC2 | Zinc finger, DHHC-type containing 2 |
| 243528_at | | |
| 201531_at | ZFP36 | zinc finger protein 36, C3H type, homolog (mouse) |
| 218349_s_at; 222606_at | ZWILCH | Zwilch, kinetochore associated, homolog (Drosophila) |

The Brd4 signature for the Dutch Rosetta cohort is generated by matching the gene symbols from the mouse dataset to the published Hu25K chip annotation files.

Analysis of tumor gene expression from breast cancer datasets is performed using BRB ArrayTools. Affymetrix datasets are downloaded from the NCBI Gene Expression Omnibus (GEO). The Dutch data set is downloaded from the Rosetta Company website. Expression data are loaded into BRB ArrayTools using the Affymetrix GeneChip Probe Level Data option or the Data Import Wizard. Data are filtered to exclude any probe set that is not a component of the Brd4 signature, and to eliminate any probe set whose expression variation across the data set was P>0.01.

The resulting gene signature for the five data sets consequently varies from 235-346 probe sets. Human BRD4 profiles are then used for unsupervised clustering of publicly available datasets into two groups representing high and low levels of BRD4 activation in patient samples. Specifically, unsupervised clustering of each dataset is performed using the Samples Only clustering option of BRB ArrayTools. Clustering is performed using average linkage, the centered correlation metric and center the genes analytical option. Samples are assigned into two groups based on the first bifurcation of the cluster dendogram, and Kaplan-Meier survival analysis performed using the Survival module of the software package Statistica to investigate whether there was a survival difference between the two groups. Significance of survival analyses is performed using the Cox F-test.

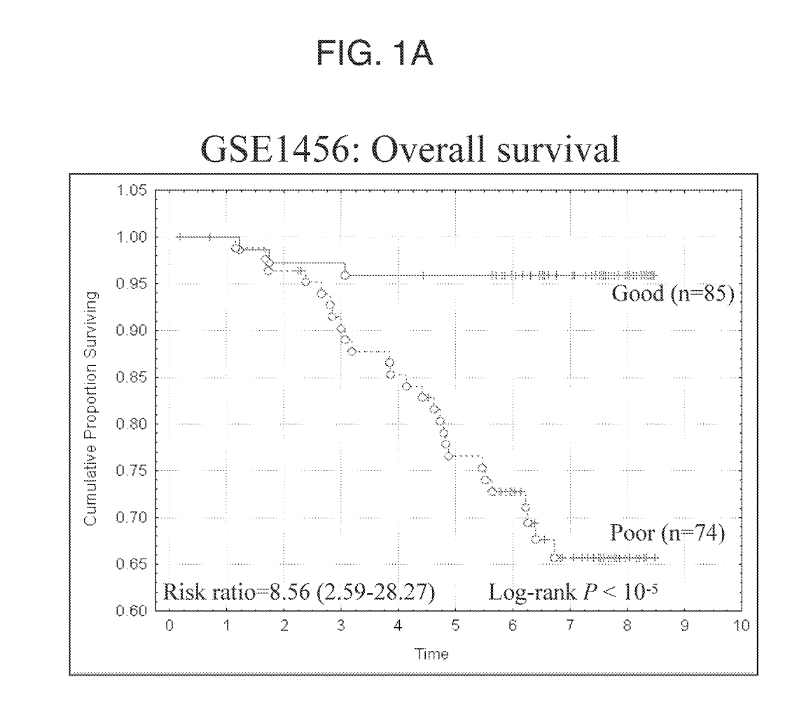

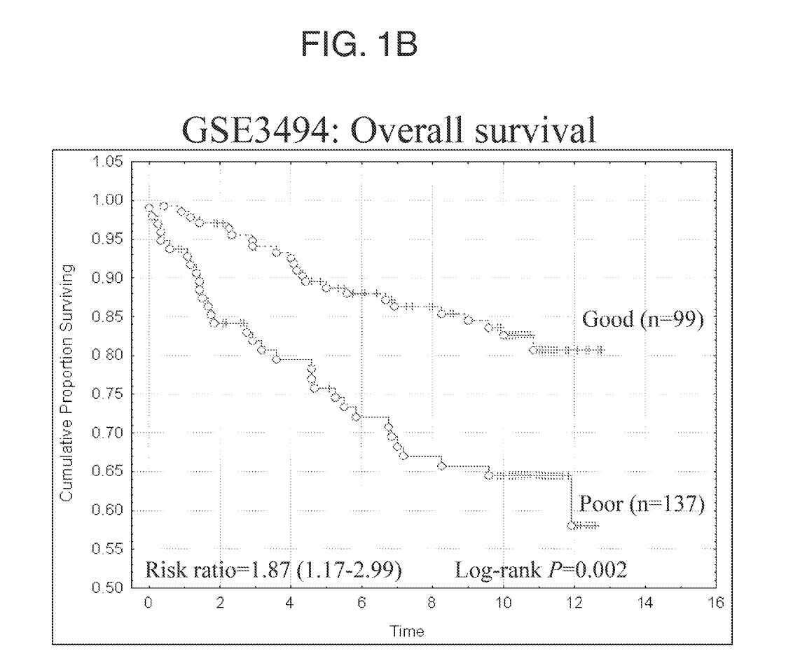

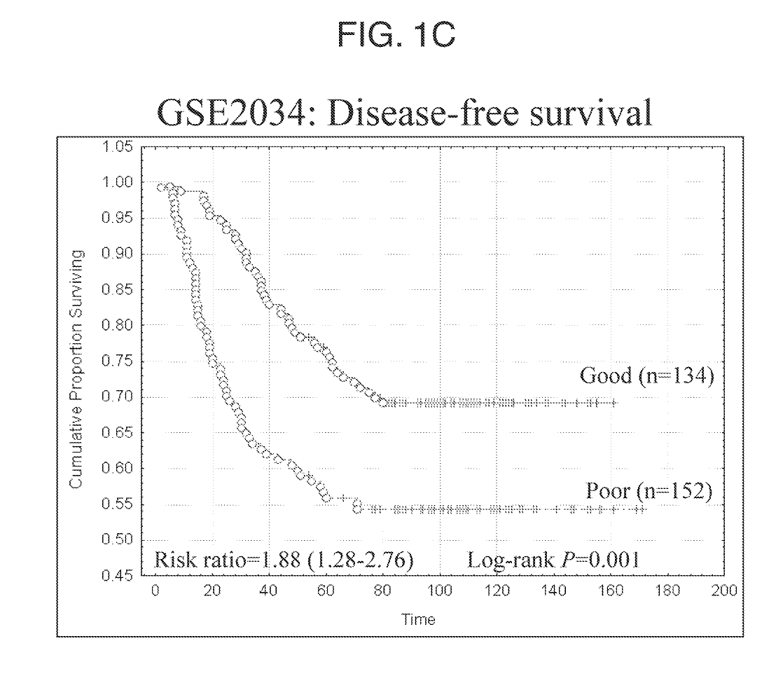

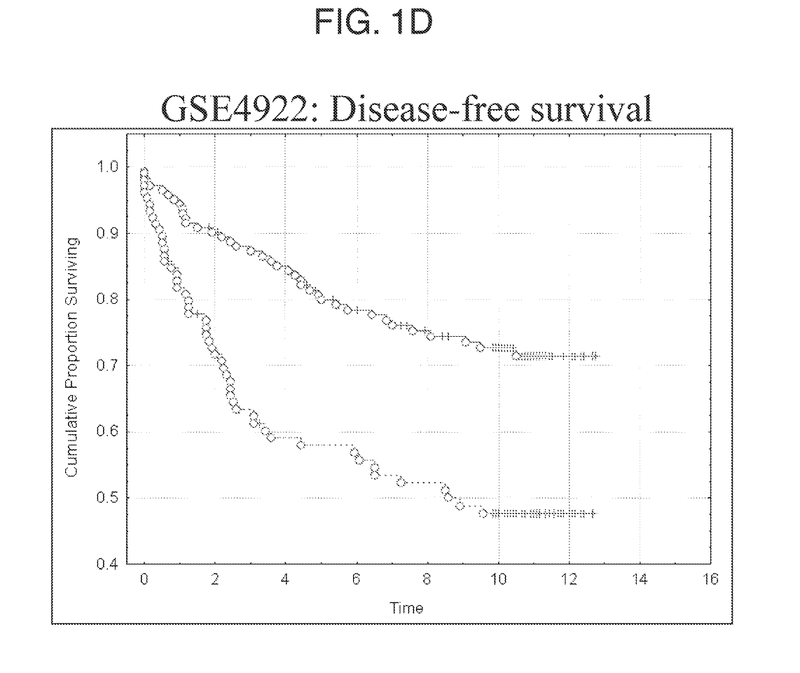

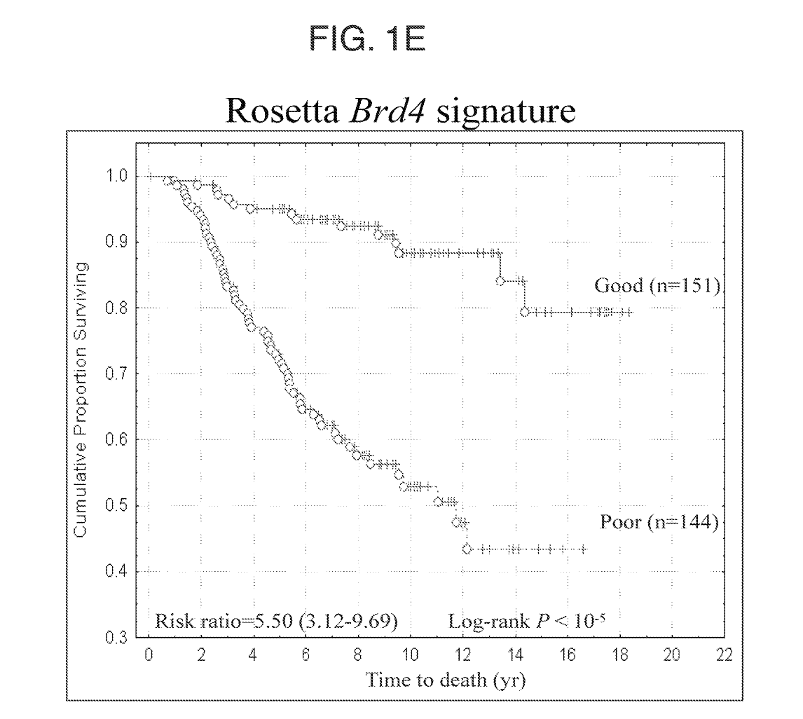

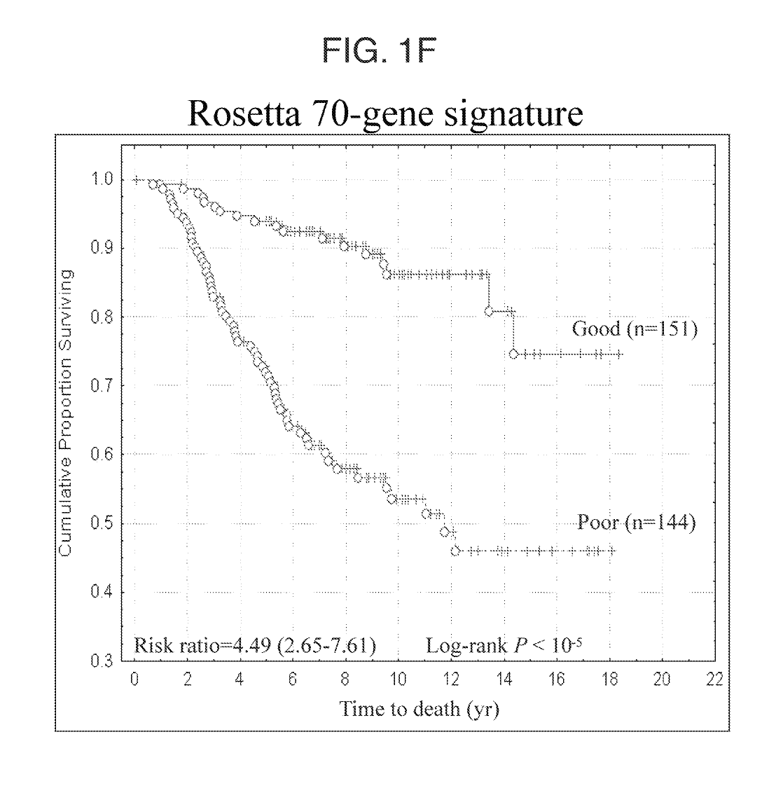

The Brd4 signature consistently and robustly predicts survival and/or relapse in four separate breast cancer microarray datasets performed on Affymetrix GeneChips. A significant difference in the overall likelihood of survival is observed in the GSE1456 dataset with 8-year survival being 95.9% vs. 65.5% for the good and poor prognosis Brd4 signatures, respectively (FIG. 1A). A similar effect is observed in the GSE3494 dataset with 12-year survival being 80.6% vs. 57.5% for the good and poor prognosis Brd4 signatures, respectively (FIG. 1B). The endpoint for the GSE2034 and GSE4922 differ in that disease-free survival is measured. A similar effect is seen in both cohorts with 10-year disease free survival being 68.9% vs. 54.2% in the GSE2034 dataset (FIG. 1C), and 71.3% vs. 47.6% in the GSE4922 dataset (FIG. 1D) for the good and poor prognosis Brd4 signatures, respectively.

The Brd4 signature is also highly predictive of overall survival in the Dutch Rosetta dataset, with the overall survival being estimated to be 78.5% vs. 45.1% for the good and poor prognosis Brd4 signatures, respectively (Brd4 signature hazard ratio=5.50, 95% confidence interval [CI]=3.12-9.69; FIG. 1E). Indeed, it would appear that the Brd4 signature possesses a slightly greater ability to predict survival in this dataset than the 70-gene signature described by van't Veer et al (van't Veer et al., Nature 415: 530-536 (2002); FIG. 1F). Specifically, the survival for the good and poor prognosis 70-gene signatures are estimated to be 72.6% vs. 47.0%, respectively (70 gene signature hazard ratio=4.49, 95% CI=2.65-7.61).

Characterization of Brd4 signature genes associate with survival in each of the breast cancer datasets reveal overlapping, but not identical gene expression signatures (Table 8).

TABLE 8

| | | Hazard Ratio | | | | | |
|---|---|---|---|---|---|---|---|
| Probe Set ID | Gene Symbol | GSE1456 | GSE2034 | GSE3494 | GSE4922 | Dutch Brd4 Sig | Dutch 70 Gene Sig |
| 208747_s_at | C1S | 0.7 | | 0.6 | 0.7 | | |
| 205022_s_at | CHES1 | 0.5 | | | | | |
| 218031_s_at | CHES1 | 0.5 | | 0.5 | 0.6 | | |
| 200810_s_at | CIRBP | 0.4 | | 0.6 | | 0.1 | |
| 200811_at | CIRBP | 0.5 | | 0.6 | 0.7 | 0.1 | |
| 214724_at | DIXDC1 | 0.4 | | 0.5 | | | |
| 215719_x_at | FAS | 0.3 | 0.8 | 0.5 | | | |
| 204781_s_at | FAS | 0.4 | | 0.5 | 0.5 | | |
| 216252_x_at | FAS | 0.4 | | 0.4 | 0.5 | | |
| 205498_at | GHR | 0.7 | | 0.7 | | | |
| 202615_at | GNAQ | 0.5 | | | | | |
| 201124_at | ITGB5 | 0.5 | | 0.6 | | 0.4 | |
| 213422_s_at | MXRA8 | 0.6 | | 0.6 | | | |
| 212448_at | NEDD4L | 0.3 | | | | | |
| 218730_s_at | OGN | 0.5 | | | | 0.3 | |
| 214177_s_at | PBXIP1 | 0.3 | | | | | |
| 221726_at | RPL22 | 0.3 | | | | 0.2 | |
| 214042_s_at | RPL22 | 0.3 | | | | 0.2 | |
| 203509_at | SORL1 | 0.3 | | 0.6 | 0.6 | 0.3 | |
| 202796_at | SYNPO | 0.3 | | 0.4 | 0.6 | | |
| 204872_at | TLE4 | 0.4 | | 0.6 | | | |
| 201010_s_at | TXNIP | 0.5 | | 0.5 | 0.6 | | |
| 201009_s_at | TXNIP | 0.5 | 0.7 | 0.6 | 0.7 | | |
| 201008_s_at | TXNIP | 0.6 | 0.7 | 0.7 | 0.7 | | |
| 218115_at | ASF1B | 3.9 | | 3.0 | 2.2 | | |
| 219918_s_at | ASPM | 1.9 | 1.4 | 1.4 | 1.3 | | |
| 202672_s_at | ATF3 | | 0.8 | | | | |
| 204092_s_at | AURKA | 2.3 | | 1.6 | 1.3 | | |
| 208079_s_at | AURKA | 1.8 | 1.5 | 1.5 | 1.4 | | |
| 209464_at | AURKB | 2.2 | | 1.6 | 1.5 | | |
| 202095_s_at | BIRC5 | 1.7 | 1.3 | 1.6 | 1.6 | 6.3 | |
| 210334_x_at | BIRC5 | 2.3 | | | | 6.3 | |
| 205733_at | BLM | | | 1.8 | | 10.4 | |
| 204531_s_at | BRCA1 | | 1.3 | | | | |
| 212949_at | BRRN1 | 3.2 | 1.2 | 3.0 | 2.3 | | |
| 209642_at | BUB1 | 2.5 | 1.5 | 1.5 | 1.4 | 8.6 | |
| 215509_s_at | BUB1 | 3.8 | | | | 8.6 | |
| 216275_at | BUB1 | | 0.8 | | | 8.6 | |
| 203755_at | BUB1B | 2.3 | | 1.7 | 1.7 | 17.1 | |
| 202763_at | CASP3 | | | 2.7 | 2.9 | | |
| 203418_at | CCNA2 | 2.9 | | 1.8 | 1.6 | 3.7 | |
| 213226_at | CCNA2 | 2.1 | 1.7 | 1.9 | 1.9 | 3.7 | |
| 214710_s_at | CCNB1 | 2.3 | | 1.9 | 1.7 | 11.8 | |
| 202705_at | CCNB2 | 2.8 | 1.4 | 2.1 | 1.8 | 12.3 | |
| 205034_at | CCNE2 | 1.5 | 1.5 | 1.5 | 1.4 | 8.2 | 8.2 |
| 211814_s_at | CCNE2 | 2.2 | | 2.0 | 2.1 | 8.2 | 8.2 |
| 202870_s_at | CDC20 | 1.8 | 1.3 | 1.5 | 1.4 | 11.8 | |
| 201853_s_at | CDC25B | 2.1 | | 1.7 | 1.5 | 8.8 | |
| 1570624_at | CDC25C | | | | | 5.6 | |
| 204126_s_at | CDC45L | 4.1 | | 2.5 | 2.6 | 15.8 | |
| 203967_at | CDC6 | 1.9 | | | 1.3 | 2.9 | |
| 203968_s_at | CDC6 | 1.8 | | | | 2.9 | |
| 204510_at | CDC7 | 1.9 | | | | | |
| 221436_s_at | CDCA3 | 2.2 | 1.2 | | | | |
| 221520_s_at | CDCA8 | 2.5 | 1.2 | 1.8 | 1.7 | | |
| 209714_s_at | CDKN3 | 2.5 | | 2.1 | 1.9 | 11.4 | |
| 204962_s_at | CENPA | 1.7 | 1.4 | 1.5 | 1.4 | 8.7 | 8.7 |
| 205046_at | CENPE | 2.9 | 1.3 | 1.8 | 1.5 | 2.9 | |
| 207828_s_at | CENPF | 2.1 | 1.3 | 1.5 | 1.4 | 5.1 | |
| 209172_s_at | CENPF | 2.2 | | 1.6 | 1.5 | 5.1 | |
| 205393_s_at | CHEK1 | 2.5 | | | | 6.3 | |
| 205394_at | CHEK1 | 2.4 | | | 1.7 | 6.3 | |
| 204233_s_at | CHKA | 2.3 | | | | | |
| 218252_at | CKAP2 | | | 2.0 | 2.1 | 3.1 | |
| 204170_s_at | CKS2 | 1.5 | | 1.6 | 1.5 | 3.4 | |
| 201572_x_at | DCTD | | | 0.3 | | | |
| 210137_s_at | DCTD | | | 0.4 | | | |
| 202887_s_at | DDIT4 | | | 1.6 | 1.4 | | |
| 203764_at | DLG7 | 2.2 | 1.6 | 1.5 | 1.4 | | |
| 213647_at | DNA2L | | | 2.3 | 2.1 | 6.3 | |
| 204817_at | ESPL1 | 3.5 | | 2.5 | 2.3 | | |
| 38158_at | ESPL1 | 3.7 | | 3.1 | 2.8 | | |
| 216375_s_at | ETV5 | | 0.8 | | | | |
| 204603_at | EXO1 | 4.3 | | | | 16.6 | |
| 209692_at | EYA2 | 2.0 | | | | | |
| 203358_s_at | EZH2 | 1.8 | 1.4 | | 1.5 | 12.7 | |
| 204780_s_at | FAS | | | 0.5 | 0.7 | | |
| 218875_s_at | FBXO5 | 2.1 | | 1.8 | 1.7 | | |
| 204767_s_at | FEN1 | 2.6 | | 1.7 | 1.8 | 26.3 | |
| 204768_s_at | FEN1 | 2.4 | | 1.7 | | 26.3 | |
| 209189_at | FOS | | | 0.7 | 0.8 | 0.4 | |
| 203725_at | GADD45A | | | 0.4 | | | |
| 203178_at | GATM | | | 0.5 | 0.6 | | |
| 216733_s_at | GATM | 1.6 | | 0.5 | 0.7 | | |
| 213094_at | GPR126 | | | | 1.3 | | |
| 209398_at | HIST1H1C | 1.4 | | 1.2 | 1.2 | | |
| 206074_s_at | HMGA1 | 2.2 | | 2.6 | 2.0 | | |
| 210457_x_at | HMGA1 | | 0.8 | | | | |
| 208808_s_at | HMGB2 | | | 1.9 | 1.7 | | |
| 207165_at | HMMR | 1.7 | 1.6 | 1.6 | 1.8 | 6.9 | |
| 209709_s_at | HMMR | 2.1 | | 2.3 | 2.1 | 6.9 | |
| 205543_at | HSPA4L | 2.3 | | | | | |
| 204444_at | KIF11 | 1.5 | | 1.6 | 1.5 | | |
| 221258_s_at | KIF18A | | | 2.2 | 2.0 | | |
| 218755_at | KIF20A | 3.1 | | 1.9 | 1.6 | | |
| 216969_s_at | KIF22 | 2.7 | | | | | |
| 204709_s_at | KIF23 | 2.5 | 1.3 | 2.2 | 1.8 | | |
| 209408_at | KIF2C | 2.3 | | 1.8 | 1.6 | | |
| 211519_s_at | KIF2C | 3.3 | | 2.2 | 1.8 | | |
| 204162_at | KNTC2 | | | 1.6 | 1.4 | | |
| 201088_at | KPNA2 /// LOC643995 | 2.0 | | 1.6 | 1.6 | | |
| 211762_s_at | KPNA2 /// LOC643995 | 1.9 | | 1.4 | 1.5 | | |
| 203041_s_at | LAMP2 | 2.3 | | | | 4.1 | |
| 221581_s_at | LAT2 | | | 0.4 | | | |
| 202726_at | LIG1 | 3.3 | | 2.0 | 2.0 | | |
| 202736_s_at | LSM4 | 1.5 | | 1.7 | 1.4 | 5.8 | |
| 202737_s_at | LSM4 | 1.6 | | 2.0 | 1.6 | 5.8 | |
| 219588_s_at | LUZP5 | 3.3 | | 1.9 | 1.9 | | |
| 203362_s_at | MAD2L1 | 1.7 | 1.5 | 1.6 | 1.5 | 7.6 | |
| 201555_at | MCM3 | 2.5 | | 1.7 | 1.6 | 68.0 | |
| 212141_at | MCM4 | 2.9 | | 2.1 | 1.8 | | |
| 212142_at | MCM4 | 3.7 | | | | | |
| 222036_s_at | MCM4 | 1.9 | | 1.7 | 1.5 | | |
| 222037_at | MCM4 | 2.1 | | 1.8 | 1.5 | | |
| 201755_at | MCM5 | 2.6 | | 1.8 | 1.7 | 11.9 | |
| 216237_s_at | MCM5 | | | | 1.6 | 11.9 | |
| 201930_at | MCM6 | 2.2 | | 1.6 | 1.7 | 15.2 | 15.2 |
| 204825_at | MELK | 2.1 | 1.4 | 1.7 | 1.6 | | |
| 212020_s_at | MKI67 | 2.0 | | 1.6 | 1.5 | 11.8 | |
| 212021_s_at | MKI67 | 3.0 | | 3.0 | 2.2 | 11.8 | |
| 212022_s_at | MKI67 | 2.3 | 1.3 | 2.0 | 1.6 | 11.8 | |
| 212023_s_at | MKI67 | | | 1.8 | 1.9 | 11.8 | |
| 218883_s_at | MLF1IP | 1.9 | | 1.7 | 1.6 | | |
| 205395_s_at | MRE11A | | 1.3 | | | | |
| 204101_at | MTM1 | | 1.2 | | | 0.02 | |
| 204641_at | NEK2 | 2.0 | 1.6 | 1.5 | 1.4 | 12.2 | |
| 211080_s_at | NEK2 | 4.3 | | | | 12.2 | |
| 201577_at | NME1 | 1.8 | | | 1.5 | | |
| 204501_at | NOV | | | 0.2 | 0.4 | | |
| 214321_at | NOV | | | 0.5 | 0.6 | | |
| 212247_at | NUP205 | 2.0 | | | | | |
| 202188_at | NUP93 | 4.6 | | 1.8 | 1.7 | | |
| 218039_at | NUSAP1 | 2.4 | | 1.8 | 1.8 | | |
| 219978_s_at | NUSAP1 | 1.9 | | 1.6 | 1.5 | | |
| 219148_at | PBK | 1.7 | 1.4 | 1.3 | 1.3 | | |
| 207838_x_at | PBXIP1 | | 0.8 | | | | |
| 202240_at | PLK1 | 3.3 | | 2.7 | 2.1 | | |
| 204886_at | PLK4 | | 1.3 | | | | |
| 204887_s_at | PLK4 | | | | 1.9 | | |
| 203422_at | POLD1 | | | | | 72.8 | |
| 205909_at | POLE2 | 3.2 | | 2.0 | 1.7 | 20.8 | |
| 210809_s_at | POSTN | | 1.3 | 0.7 | | | |
| 214981_at | POSTN | | 1.1 | | | | |
| 218009_s_at | PRC1 | 2.1 | 1.5 | 1.6 | 1.6 | 16.7 | 16.7 |
| 207505_at | PRKG2 | | 0.8 | | | | |
| 220892_s_at | PSAT1 | 2.7 | 0.8 | | | | |
| 203554_x_at | PTTG1 | 2.1 | | 2.0 | 1.8 | 27.4 | |
| 222077_s_at | RACGAP1 | 2.1 | | 2.2 | 1.9 | | |
| 205024_s_at | RAD51 | 5.7 | 1.4 | 3.5 | 3.0 | 30.3 | |
| 204146_at | RAD51AP1 | 1.8 | 1.4 | | | | |
| 206499_s_at | RCC1 | 4.4 | | 3.1 | 2.3 | | |
| 204023_at | RFC4 | 1.7 | | 1.5 | 1.6 | 12.5 | 12.5 |
| 201476_s_at | RRM1 | 1.7 | | | | 7.5 | |
| 201890_at | RRM2 | 1.8 | 1.4 | 1.7 | 1.6 | 5.6 | |
| 209773_s_at | RRM2 | 2.2 | 1.4 | 1.6 | 1.6 | 5.6 | |
| 203789_s_at | SEMA3C | | | 0.7 | | 0.3 | |
| 219493_at | SHCBP1 | 3.0 | 1.5 | 2.3 | 2.0 | | |
| 203625_x_at | SKP2 | | | 1.6 | 1.4 | | |
| 204240_s_at | SMC2L1 | 2.0 | | | | 3.6 | |
| 213253_at | SMC2L1 | | | 3.3 | 2.6 | 3.6 | |
| 201663_s_at | SMC4L1 | 2.2 | | | | 3.3 | |
| 201664_at | SMC4L1 | 1.8 | | 1.6 | 1.6 | 3.3 | |
| 203145_at | SPAG5 | 2.2 | 1.3 | 2.6 | 2.2 | | |
| 209891_at | SPBC25 | 1.8 | | 4.3 | 2.6 | | |
| 205542_at | STEAP1 | | | 0.4 | 0.6 | | |
| 200783_s_at | STMN1 | 1.9 | | 1.6 | 1.6 | 10.5 | |
| 218308_at | TACC3 | 2.4 | 1.2 | 2.4 | 2.2 | 13.8 | |
| 206943_at | TGFBR1 | | 0.8 | | | | |
| 203046_s_at | TIMELESS | 2.3 | | 2.6 | 2.2 | 35.6 | |
| 202338_at | TK1 | 2.0 | | 1.9 | 1.9 | 8.1 | |
| 201291_s_at | TOP2A | 1.4 | 1.2 | 1.3 | 1.3 | 5.0 | |
| 201292_at | TOP2A | 1.7 | 1.3 | 1.4 | 1.4 | 5.0 | |
| 237469_at | TOP2A | | | | | 5.0 | |
| 202633_at | TOPBP1 | | | | 2.0 | 11.1 | |
| 210052_s_at | TPX2 | 1.9 | | 1.7 | 1.5 | | |
| 218145_at | TRIB3 | 2.1 | | 2.2 | 1.7 | | |
| 204033_at | TRIP13 | 1.8 | | 1.9 | 1.6 | 16.8 | |
| 204649_at | TROAP | | | 2.9 | 2.4 | 160.9 | |
| 204822_at | TTK | 1.4 | 1.4 | 1.5 | 1.3 | 6.3 | |
| 202954_at | UBE2C | 2.1 | | 2.0 | 1.7 | | |
| 216228_s_at | WDHD1 | | 1.3 | | | | |
| 209052_s_at | WHSC1 | 2.4 | | | | | |
| 209053_s_at | WHSC1 | 2.0 | | 1.8 | 1.9 | | |
| 209054_s_at | WHSC1 | | | | 2.0 | | |
| 221785_at | WIZ | | 0.8 | | | | |
| 219312_s_at | ZBTB10 | | 1.5 | | | | |
| 218349_s_at | ZWILCH | | | 2.0 | 2.2 | | |

| Brd4 Signature Genes Predictive only in Dutch Cohort | Hazard Ratio |
|---|---|
| ANLN | 6.3 |
| CAD | 12.3 |
| CBL | 14.3 |
| CDKN2D | 8.0 |
| CENPF | 5.1 |
| CIRBP | 0.1 |
| CP | 2.0 |
| DHODH | 16.4 |
| DLEU2 | 13.5 |
| FIGNL1 | 9.3 |
| FXYD5 | 6.2 |
| H6PD | 0.1 |
| ITGB5 | 0.4 |
| LIPG | 3.6 |
| LRP8 | 4.3 |
| NFIL3 | 5.8 |
| OGN | 0.3 |
| PLEK2 | 5.5 |
| POLE | 0.4 |
| PRIM1 | 4.8 |
| RBL1 | 17.2 |
| RPL22 | 0.2 |
| SORL1 | 0.3 |
| TACC3 | 13.8 |

The vast majority of Brd4 signature probes are predictive of survival in at least two of the four Affymetrix cohorts, and hazard ratios displayed the same directionality of effect for over 99% of probes when a probe is predictive of survival in more than one cohort. The Dutch Rosetta cohort does have a number of unique predictive signature genes. Such variations likely reflect microarray platform differences, as well as population and tumor heterogeneity. Nevertheless, it is argued that in view of the overlapping nature of the Brd4 signatures in the five cohorts, as well as the finding that the Brd4 signature is the only consistent predictor of outcome on multivariate Cox proportional analysis in all of the cohorts (Table 9), that the net effect of the Brd4 signature is both consistent and robust. Table 8 lists the Brd4 signature genes predicting survival in all 5 human breast cancer cohorts.

TABLE 9

| | GSE2034 | | GSE3934 | | GSE4922 | | Rosetta | |
|---|---|---|---|---|---|---|---|---|
| | Risk | | Risk | | Risk | | Risk | |
| | ratio (95% CI) | P | ratio (95% CI) | P | ratio (95% CI) | P | ratio (95% CI) | P |
| Brd4 signature | 2.05 (1.37-3.07) | 0.0005 | 1.86 (1.06-3.27) | 0.0300 | 2.04 (1.30-3.20) | 0.0020 | 4.44 (2.42-8.12) | <0.0001 |
| Lymph node status | * | * | 2.74 (1.56-4.82) | 0.0004 | 1.49 (0.95-2.32) | 0.0800 | 1.09 (0.87-1.37) | 0.4400 |
| Tumor ER expression | 1.15 (0.91-1.44) | 0.2313 | 1.50 (0.62-3.59) | 0.3700 | 1.22 (0.65-2.30) | 0.5300 | 1.39 (1.09-1.77) | 0.0080 |
| Tumor size (<=2 cm) | * | * | 1.63 (1.15-2.30) | 0.0060 | 1.31 (1.03-1.67) | 0.0290 | 1.27 (0.80-1.97) | 0.3200 |
| 70 Gene Rosetta | * | * | * | * | * | * | 1.3 (0.79-2.02) | 0.3200 |
| Signature | | | | | | | | |

* Data not available for this cohort

This example demonstrated that the expression levels of the target molecules of Table 8 correlate with cancer survival.

### Example 3

This example demonstrates that the Brd4 signature sub-stratifies patients with node-negative and ER-positive primary tumors into good and poor outcome groups based on tumor gene expression.

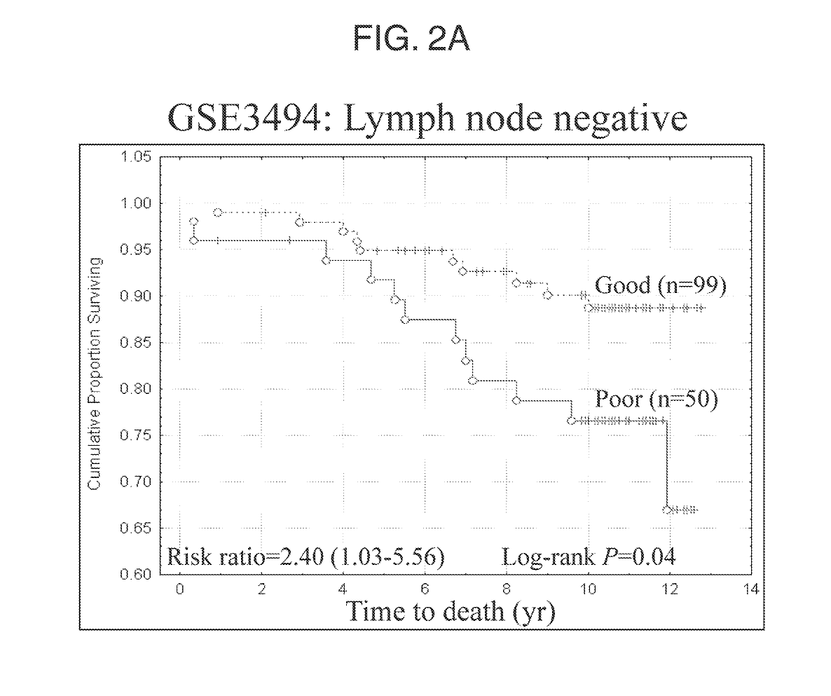

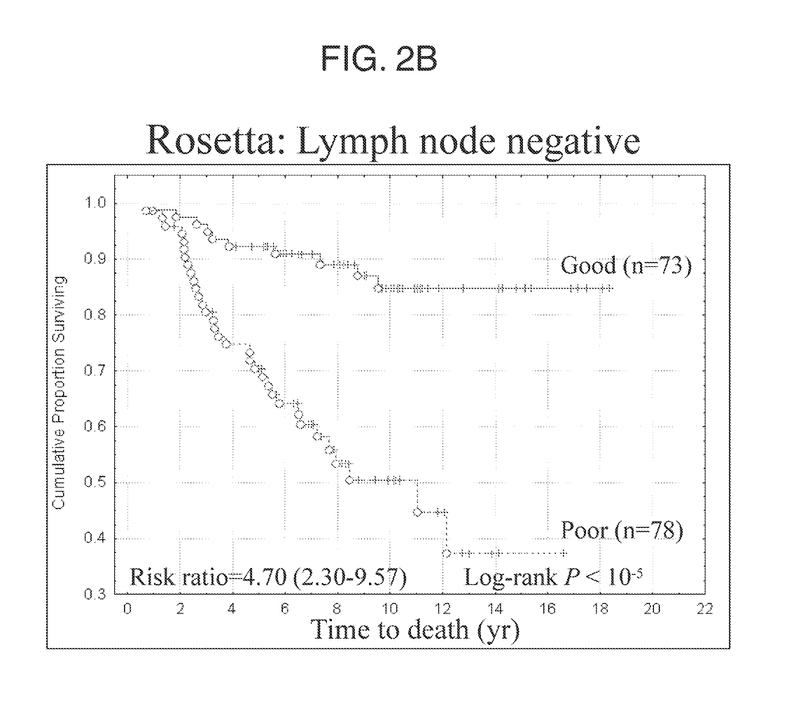

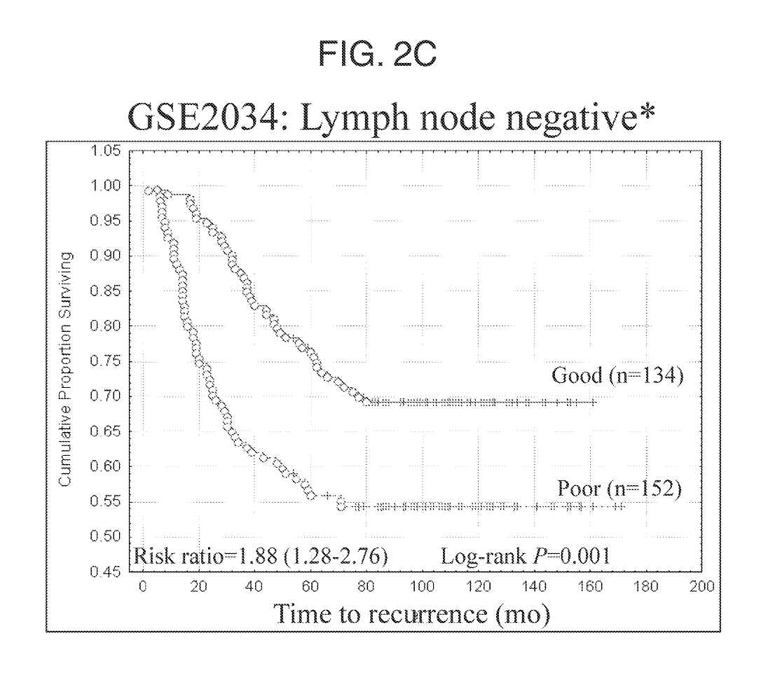

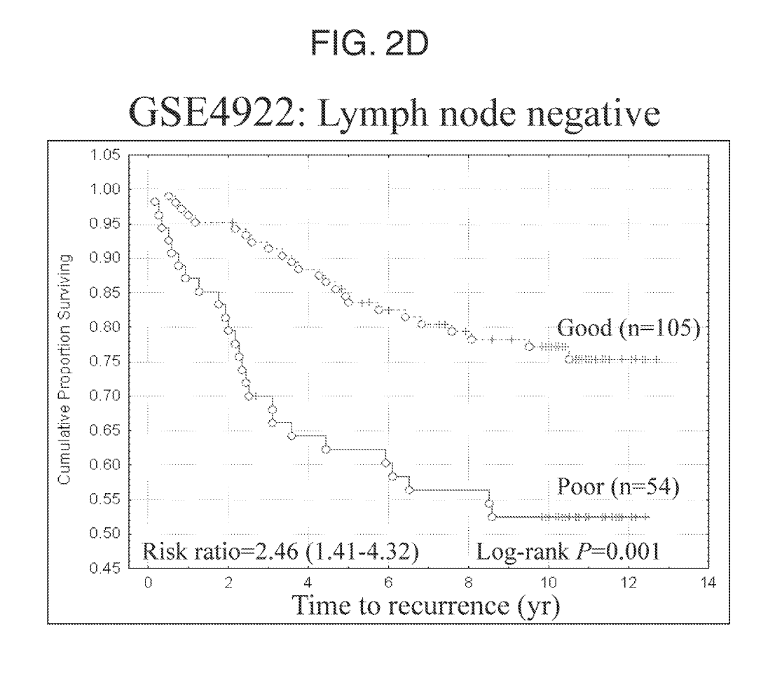

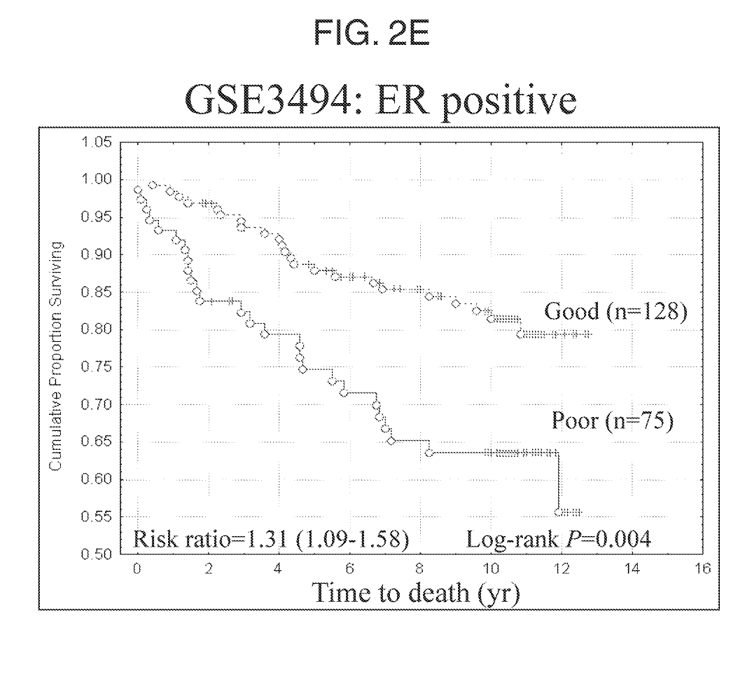

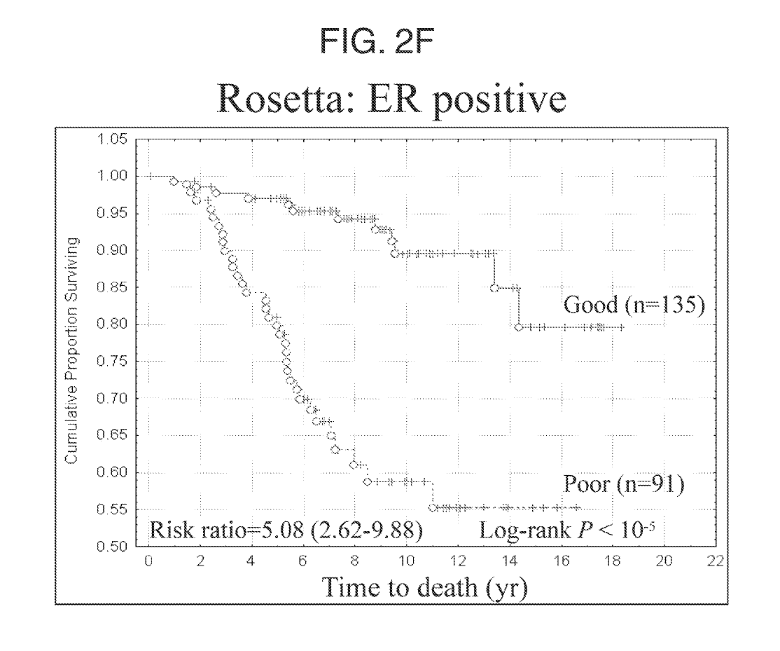

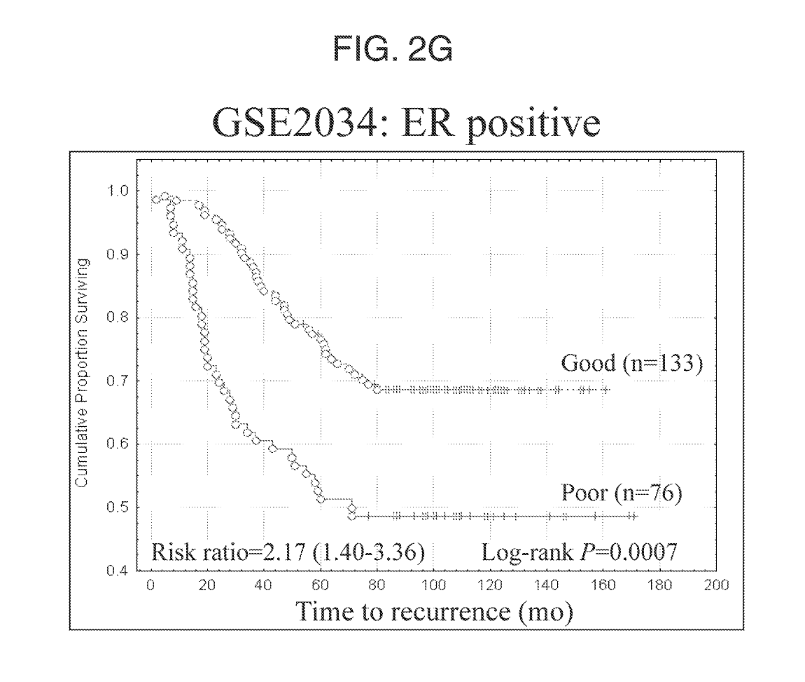

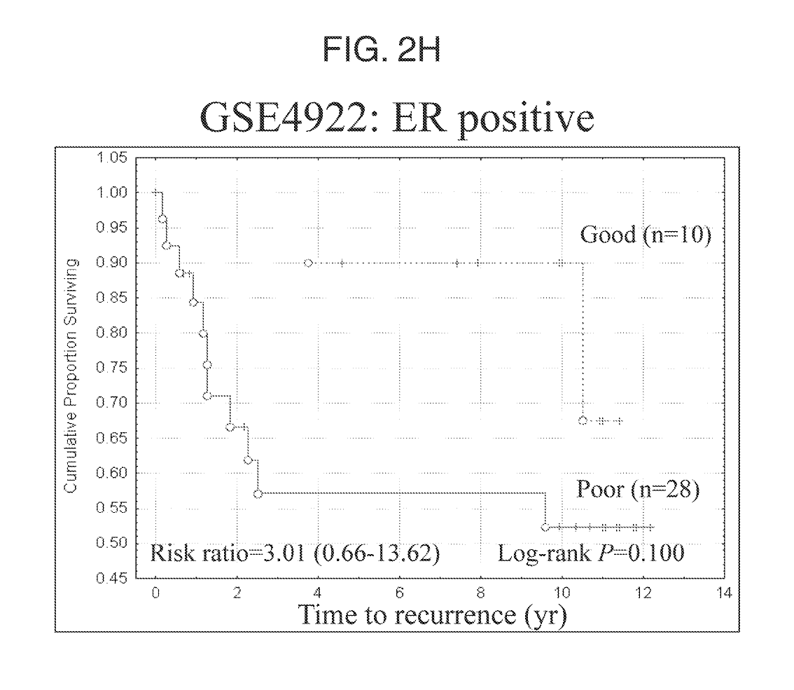

The effect of the Brd4 signature gene expression upon survival in node-negative patients is determined when clinical data are available. Signature gene expression has a modest but statistically significant effect upon survival in GSE3494 node-negative patients, with overall 12-year survival being 88.0% in the good prognosis group and 66.8% in the poor prognosis group (FIG. 2A). A more dramatic effect is observed in the other three node-negative datasets. Overall survival in the Dutch Rosetta node-negative patients is 83.9% vs. 38.5% for the good and poor prognosis Brd4 signatures, respectively (FIG. 2B). Similar effects are seen in the GSE2034 lymph node negative dataset with 10-year disease free survival being 68.9% vs. 54.2% in the good and poor prognosis Brd4 signatures, respectively (FIG. 2C), and in GSE4922 node-negative patients being 75.3% vs. 52.3% for the good and poor prognosis Brd4 signatures, respectively (FIG. 2D).

A similar stratification effect by tumor Brd4 signature gene expression is observed in ER-positive patients when sufficient clinical data are available. Signature gene expression has a modest but statistically significant effect upon survival in GSE3494 ER-positive patients, with overall 12-year survival being 79.3% in the good prognosis group and 54.3% in the poor prognosis group (FIG. 2E). Signature gene expression has a stronger effect in two of the three ER-positive datasets, with an overall survival in the Dutch Rosetta ER-positive patients being 78.4% vs. 54.4% for the good and poor prognosis Brd4 signatures, respectively (FIG. 2F). Furthermore, disease-free survival in GSE2034 ER-positive patients is estimated as being 68.4% vs. 48.5% for the good and poor prognosis Brd4 signatures, respectively (FIG. 2G). The GSE4922 dataset contains insufficient numbers of ER positive subjects and are subsequently too underpowered to detect any significant effect of signature gene expression upon disease-free survival (FIG. 2H).

This example demonstrated that detection of the gene expression levels of genes of Table 8 correlate with certain tumor characteristics.

### Example 4

This example demonstrates the microarray analysis of mouse Mvt-1 cell lines ectopically expressing Anakin.

Affymetrix microarrays are used to compare gene expression in four Mvt-1/Anakin clonal isolates and three Mvt-1/β-galactosidase clonal isolates. An Anakin expression signature is identified using the Class Comparison tool of BRB ArrayTools is performed, using a two-sample t-test with random variance univariate test. P-values for significance are computed based on 10,000 random permutations, at a nominal significance level of each univariate test of 0.0001. A total of 1,739 probe sets representing 1346 genes passed these conditions. Examples of significantly up-regulated and down-regulated probes according to these criteria are listed in Tables 10 and 11, respectively.

TABLE 10

| | Fold difference of | | | |
|---|---|---|---|---|
| | geom. means | | | |
| | (control/transfected | | | |
| | cell lines) | Probe Set ID | Gene Symbol | Description |
| 1 | 59.880 | 1453275_at | 2310002L13Rik | RIKEN cDNA 2310002L13 gene |
| 2 | 45.370 | 1422011_s_at | Xlr /// | X-linked lymphocyte-regulated complex /// RIKEN cDNA 3830403N18 gene |
| | | | 3830403N18Rik | |
| 3 | 35.231 | 1440557_at | Ipw | imprinted gene in the Prader-Willi syndrome region |
| 4 | 32.555 | 1426181_a_at | Il24 | interleukin 24 |
| 5 | 18.132 | 1426615_s_at | Ndrg4 | N-myc downstream regulated gene 4 |
| 6 | 16.046 | 1436188_a_at | Ndrg4 | N-myc downstream regulated gene 4 |
| 7 | 14.663 | 1456326_at | Gm784 | gene model 784, (NCBI) |
| 8 | 13.938 | 1450871_a_at | Bcat1 | branched chain aminotransferase 1, cytosolic |
| 9 | 12.981 | 1419082_at | Serpinb2 | serine (or cysteine) proteinase inhibitor, clade B, member 2 |
| 10 | 12.742 | 1451791_at | Tfpi | tissue factor pathway inhibitor |
| 11 | 12.488 | 1426851_a_at | Nov | nephroblastoma overexpressed gene |
| 12 | 12.135 | 1420310_at | | |
| 13 | 11.476 | 1426852_x_at | Nov | nephroblastoma overexpressed gene |
| 14 | 11.333 | 1452367_at | Coro2a | coronin, actin binding protein 2A |
| 15 | 11.260 | 1421979_at | Phex | phosphate regulating gene with homologies to endopeptidases on the |
| | | | | X chromosome |
| | | | | (hypophosphatemia, vitamin D resistant rickets) |
| 16 | 10.722 | 1416295_a_at | Il2rg | interleukin 2 receptor, gamma chain |
| 17 | 10.426 | 1443653_at | D930038M13Rik | RIKEN cDNA D930038M13 gene |
| 18 | 10.065 | 1424339_at | Oasl1 | 2′-5′ oligoadenylate synthetase-like 1 |
| 19 | 9.711 | 1451790_a_at | Tfpi | tissue factor pathway inhibitor |
| 20 | 9.565 | 1452679_at | 2410129E14Rik | RIKEN cDNA 2410129E14 gene |
| 21 | 9.376 | 1417267_s_at | Fkbp11 | FK506 binding protein 11 |
| 22 | 9.339 | 1421134_at | Areg | amphiregulin |
| 23 | 9.030 | 1416368_at | Gsta4 | glutathione S-transferase, alpha 4 |

TABLE 11

| | | | | |
|---|---|---|---|---|
| 1 | 0.002 | 1430162_at | 3830417A13Rik | RIKEN cDNA 3830417A13 gene |
| 2 | 0.018 | 1415983_at | Lcp1 | lymphocyte cytosolic protein 1 |
| 3 | 0.032 | 1418004_a_at | 1810009M01Rik | RIKEN cDNA 1810009M01 gene |
| 4 | 0.033 | 1448160_at | Lcp1 | lymphocyte cytosolic protein 1 |
| 5 | 0.036 | 1416666_at | Serpine2 | serine (or cysteine) proteinase inhibitor, clade E, member 2 |
| 6 | 0.043 | 1450678_at | Itgb2 | integrin beta 2 |
| 7 | 0.045 | 1423909_at | 0610011I04Rik | RIKEN cDNA 0610011I04 gene |
| 8 | 0.049 | 1418664_at | Mpdz | multiple PDZ domain protein |
| 9 | 0.058 | 1417848_at | MGI: 2180715 | glucocorticoid induced gene 1 |
| 10 | 0.062 | 1453152_at | Mamdc2 | MAM domain containing 2 |
| 11 | 0.063 | 1434442_at | D5Ertd593e | DNA segment, Chr 5, ERATO Doi 593, expressed |
| 12 | 0.063 | 1428891_at | 9130213B05Rik | RIKEN cDNA 9130213B05 gene |
| 13 | 0.066 | 1426858_at | Inhbb | inhibin beta-B |
| 14 | 0.068 | 1434465_x_at | Vldlr | very low density lipoprotein receptor |
| 15 | 0.073 | 1450107_a_at | Renbp | renin binding protein |
| 16 | 0.074 | 1448303_at | Gpnmb | glycoprotein (transmembrane) nmb |
| 17 | 0.075 | 1417061_at | Slc40a1 | solute carrier family 40 (iron-regulated transporter), member 1 |
| 18 | 0.088 | 1451461_a_at | Aldoc | aldolase 3, C isoform |
| 19 | 0.090 | 1434920_a_at | Evl | Ena-vasodilator stimulated phosphoprotein |
| 20 | 0.094 | 1421063_s_at | Snrpn /// Snurf | small nuclear ribonucleoprotein N /// SNRPN upstream |
| | | | | reading frame |
| 21 | 0.097 | 1450044_at | Fzd7 | frizzled homolog 7 (Drosophila) |
| 22 | 0.100 | 1416855_at | Gas1 | growth arrest specific 1 |
| 23 | 0.104 | 1434372_at | | |
| 24 | 0.106 | 1436838_x_at | Cotl1 | coactosin-like 1 (Dictyostelium) |
| 25 | 0.112 | 1420851_at | Pard6g | par-6 partitioning defective 6 homolog gamma (C. elegans) |
| 26 | 0.116 | 1449896_at | Mlph | melanophilin |
| 27 | 0.116 | 1417900_a_at | Vldlr | very low density lipoprotein receptor |
| 28 | 0.119 | 1434191_at | A530016O06Rik | RIKEN cDNA A530016O06 gene |
| 29 | 0.124 | 1450455_s_at | Akr1c12 | aldo-keto reductase family 1, member C12 |
| 30 | 0.125 | 1445597_s_at | Hrasls3 | HRAS like suppressor 3 |
| 31 | 0.127 | 1418910_at | Bmp7 | bone morphogenetic protein 7 |

A human Anakin gene expression signature is generated by mapping the differentially regulated genes from mouse array data to human Rosetta probe set annotations (van't Veer et al., Nature 415: 530-536 (2002)). One hundred and ninety six genes from the mouse data can be mapped to the available Rosetta Hu25K chip annotations. The 295 samples of the Rosetta data set (van't Veer et al., 2002, supra) are clustered into one of two groups representing high and low levels of Anakin activation in primary tumor samples in an unsupervised manner based on the 196 significantly differentially expressed Anakin signature genes on the Hu25K chip.

Of the 196 genes, 33 genes (Table 12) are identified as predictive of cancer survival in the van't Veer breast cancer cohort (van 't Veer et al., 2002, supra), 16 genes (Table 13) are identified as predictive of cancer survival in the GSE1456 breast cancer cohort, 8 genes (Table 14) are identified as predictive of cancer survival in the GSE3494 breast cancer cohort, and 3 genes (Table 15) are identified as predictive of cancer survival in the GSE4922 breast cancer cohort. The genes of Tables 12-15 correlate with the genes of Groups 1-4 of Table 1.

TABLE 12

| | Parametric p-value | FDR | Hazard Ratio | SD of log ratios | Unique id | Target Molecule |
|---|---|---|---|---|---|---|
| 1 | <1e−07 | <1e−07 | 56.154 | 0.169 | NM_001605 | AARS |
| 2 | 1.1e−05 | 0.0005325 | 5.669 | 0.275 | NM_004207 | SLC16A3 |
| 3 | 1.63e−05 | 0.0005325 | 0.125 | 0.205 | NM_001280 | CIRBP |
| 4 | 2.2e−05 | 0.000539 | 0.26 | 0.331 | NM_014246 | CELSR1 |
| 5 | 6.2e−05 | 0.0012152 | 9.327 | 0.176 | NM_003498 | SNN |
| 6 | 0.0001228 | 0.0020057 | 0.181 | 0.243 | AI819706 | Contig1951 |
| 7 | 0.0001724 | 0.0024136 | 5.296 | 0.245 | AF035284 | FADS1 |
| 8 | 0.0002729 | 0.003343 | 0.146 | 0.232 | NM_014456 | PDCD4 |
| 9 | 0.0006509 | 0.0070844 | 5.828 | 0.183 | NM_020166 | MCCC1 |
| 10 | 0.0007229 | 0.0070844 | 3.319 | 0.306 | NM_005165 | ALDOC |
| 11 | 0.0015771 | 0.0140505 | 0.219 | 0.266 | NM_000824 | GLRB |
| 12 | 0.0020862 | 0.016009 | 0.117 | 0.179 | D25304 | ARHGEF6 |
| 13 | 0.0022688 | 0.016009 | 0.38 | 0.377 | NM_000930 | PLAT |
| 14 | 0.002287 | 0.016009 | 5.716 | 0.188 | NM_003056 | SLC19A1 |
| 15 | 0.0027271 | 0.0178171 | 4.245 | 0.205 | S40706 | DDIT3 |
| 16 | 0.004977 | 0.0304841 | 2.657 | 0.282 | NM_016577 | RAB6B |
| 17 | 0.0061899 | 0.035683 | 4.603 | 0.188 | NM_001550 | IFRDI |
| 18 | 0.0067291 | 0.0366362 | 0.465 | 0.382 | NM_000931 | PLAT |
| 19 | 0.0079349 | 0.0409274 | 0.234 | 0.206 | NM_004126 | GNG11 |
| 20 | 0.0101124 | 0.0494517 | 0.294 | 0.253 | AL079298 | MCCC2 |
| 21 | 0.0105968 | 0.0494517 | 0.189 | 0.162 | NM_001560 | IL13RA1 |
| 22 | 0.0160849 | 0.0716509 | 0.245 | 0.181 | NM_003894 | PER2 |
| 23 | 0.018496 | 0.078809 | 2.035 | 0.358 | NM_001885 | CRYAB |
| 24 | 0.0219223 | 0.0895161 | 0.344 | 0.306 | NM_002147 | HOXB5 |
| 25 | 0.0242353 | 0.0950024 | 3.99 | 0.194 | AI970292 | Contig45049_RC |
| 26 | 0.0252599 | 0.0952104 | 0.297 | 0.199 | AL117599 | DKFZp564I0463 |
| 27 | 0.0297937 | 0.1081401 | 2.774 | 0.253 | NM_003234 | TFRC |
| 28 | 0.0319726 | 0.1119041 | 0.341 | 0.214 | NM_003505 | FZD1 |
| 29 | 0.0336773 | 0.113806 | 2.75 | 0.237 | NM_002298 | LCP1 |
| 30 | 0.0361845 | 0.1182027 | 0.387 | 0.241 | NM_000690 | ALDH2 |
| 31 | 0.0375725 | 0.1187776 | 2.43 | 0.165 | NM_004775 | B4GALT6 |
| 32 | 0.0408441 | 0.1248585 | 4.558 | 0.186 | NM_012257 | HBP1 |
| 33 | 0.0420442 | 0.1248585 | 4.106 | 0.164 | NM_013995 | LAMP2 |
| 34 | | | 0.3 | | NM_173872.2 | CLCN3 |
| 35 | | | 4.0 | | NM_002033.2 | FUT4 |
| 36 | | | 0.2 | | NM_014236.1 | GNPAT |

TABLE 13

| | Parametric | | Hazard | SD of log | | | | Gene |
|---|---|---|---|---|---|---|---|---|
| | p-value | FDR | Ratio | intensities | Probe set | Annotations | Description | symbol |
| 1 | 1.2e−06 | 0.0003311 | 0.223 | 0.549 | 217707_x_at | Info | SWI/SNF related, | SMARCA2 |
| | | | | | | | matrix associated, | |
| | | | | | | | actin dependent | |
| | | | | | | | regulator of | |
| | | | | | | | chromatin, subfamily | |
| | | | | | | | a, member 2 | |
| 2 | 2.2e−06 | 0.0003311 | 0.318 | 0.585 | 206542_s_at | Info | SWI/SNF related, | SMARCA2 |
| | | | | | | | matrix associated, | |
| | | | | | | | actin dependent | |
| | | | | | | | regulator of | |
| | | | | | | | chromatin, subfamily | |
| | | | | | | | a, member 2 | |
| 3 | 4.5e−06 | 0.0004515 | 5.194 | 0.399 | 201000_at | Info | alanyl-tRNA | AARS |
| | | | | | | | synthetase | |
| 4 | 4.94e−05 | 0.0030702 | 0.234 | 0.424 | 201648_at | Info | Janus kinase 1 (a | JAK1 |
| | | | | | | | protein tyrosine | |
| | | | | | | | kinase) | |
| 5 | 5.1e−05 | 0.0030702 | 4.726 | 0.452 | 219575_s_at | Info | peptide deformylase- | PDF /// |
| | | | | | | | like protein /// | COG8 |
| | | | | | | | component of | |
| | | | | | | | oligomeric golgi | |
| | | | | | | | complex 8 | |
| 6 | 7.04e−05 | 0.0033562 | 6.876 | 0.37 | 218107_at | Info | WD repeat domain 26 | WDR26 |
| 7 | 7.93e−05 | 0.0033562 | 4.621 | 0.382 | 202188_at | Info | nucleoporin 93 kDa | NUP93 |
| 8 | 8.92e−05 | 0.0033562 | 2.817 | 0.667 | 201584_s_at | Info | DEAD (Asp-Glu-Ala- | DDX39 |
| | | | | | | | Asp) box polypeptide | |
| | | | | | | | 39 | |
| 9 | 0.0001162 | 0.0038862 | 5.956 | 0.362 | 203612_at | Info | bystin-like | BYSL |
| 10 | 0.0002035 | 0.0061254 | 0.447 | 1.09 | 218087_s_at | Info | sorbin and SH3 | SORBS1 |
| | | | | | | | domain containing 1 | |
| 11 | 0.0003349 | 0.0091641 | 0.16 | 0.412 | 213306_at | Info | multiple PDZ domain | MPDZ |
| | | | | | | | protein | |
| 12 | 0.0003808 | 0.0095517 | 0.467 | 0.797 | 221748_s_at | Info | tensin 1 /// tensin 1 | TNS1 |
| 13 | 0.000467 | 0.0108128 | 0.465 | 0.809 | 212226_s_at | Info | phosphatidic acid | PPAP2B |
| | | | | | | | phosphatase type 2B | |
| 14 | 0.0007256 | 0.0156004 | 0.417 | 0.641 | 200810_s_at | Info | cold inducible RNA | CIRBP |
| | | | | | | | binding protein | |
| 15 | 0.00098 | 0.0186996 | 0.408 | 0.649 | 205251_at | Info | period homolog 2 | PER2 |
| | | | | | | | (Drosophila) | |
| 16 | 0.000994 | 0.0186996 | 0.496 | 0.944 | 209047_at | Info | aquaporin 1 (channel- | AQP1 |
| | | | | | | | forming integral | |
| | | | | | | | protein, 28 kDa) | |

TABLE 14

| | Parametric | | Hazard | SD of log | | | | Gene |
|---|---|---|---|---|---|---|---|---|
| | p-value | FDR | Ratio | intensities | Probe set | Annotations | Description | symbol |
| 1 | 1.61e−05 | 0.0047012 | 2.421 | 0.681 | 204900_x_at | Info | sin3-associated | SAP30 |
| | | | | | | | polypeptide, 30 kDa | |
| 2 | 0.0002015 | 0.0262341 | 0.321 | 0.446 | 203758_at | Info | cathepsin O | CTSO |
| 3 | 0.0002713 | 0.0262341 | 0.324 | 0.474 | 203261_at | Info | dynactin 6 | DCTN6 |
| 4 | 0.0004705 | 0.0262341 | 3.538 | 0.338 | 204899_s_at | Info | sin3-associated | SAP30 |
| | | | | | | | polypeptide, 30 kDa | |
| 5 | 0.0005355 | 0.0262341 | 0.484 | 0.714 | 204451_at | Info | frizzled homolog 1 | FZD1 |
| | | | | | | | (Drosophila) | |
| 6 | 0.0005618 | 0.0262341 | 1.644 | 0.841 | 202856_s_at | Info | solute carrier family 16 | SLC16A3 |
| | | | | | | | (monocarboxylic acid | |
| | | | | | | | transporters), member 3 | |
| 7 | 0.0006289 | 0.0262341 | 0.365 | 0.518 | 221747_at | Info | Tensin 1 /// Tensin 1 | TNS |
| 8 | 0.0007515 | 0.0274297 | 2.681 | 0.392 | 219573_at | Info | leucine rich repeat | LRRC16 |
| | | | | | | | containing 16 | |

TABLE 15

| | | % CV | | | | Gene |
|---|---|---|---|---|---|---|
| | p-value | Support | Probe set | Description | Annotations | symbol |
| 1 | 0.000494 | 97.99 | 201584_s_at | DEAD (Asp-Glu-Ala-Asp) box | Info | DDX39 |
| | | | | polypeptide 39 | | |
| 2 | 0.000701 | 94.38 | 204900_x_at | sin3-associated polypeptide, 30 kDa | Info | SAP30 |
| 3 | 0.000957 | 49.4 | 202856_s_at | solute carrier family 16 (monocarboxylic | Info | SLC16A3 |
| | | | | acid transporters), member 3 | | |

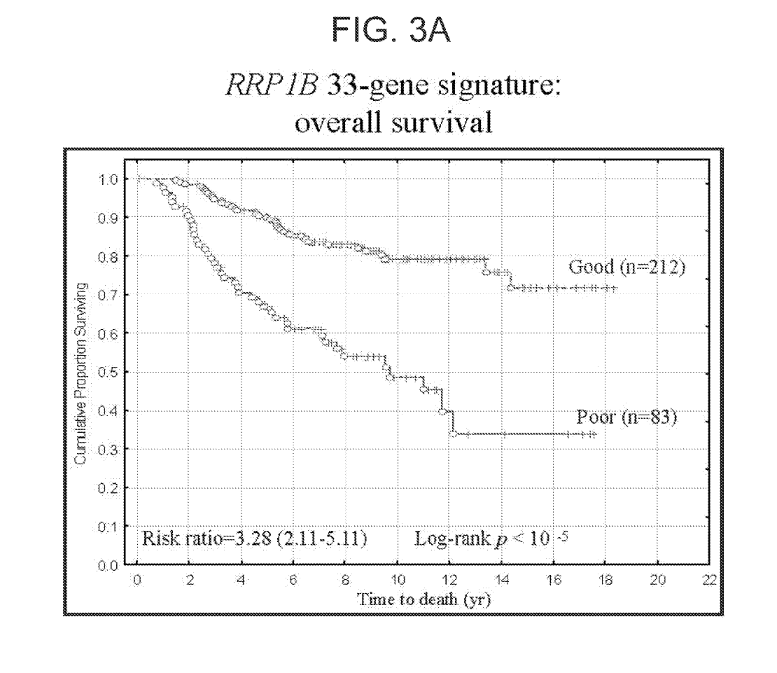

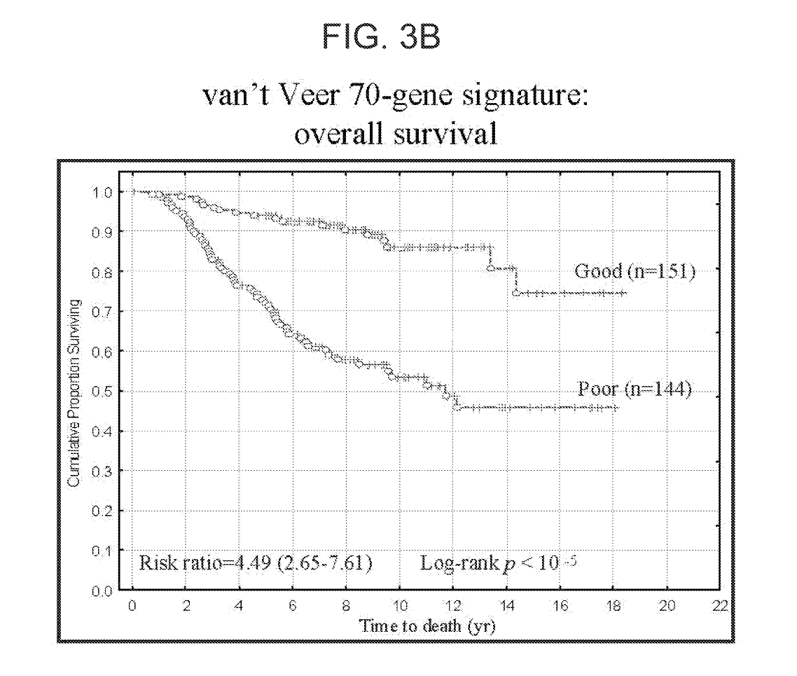

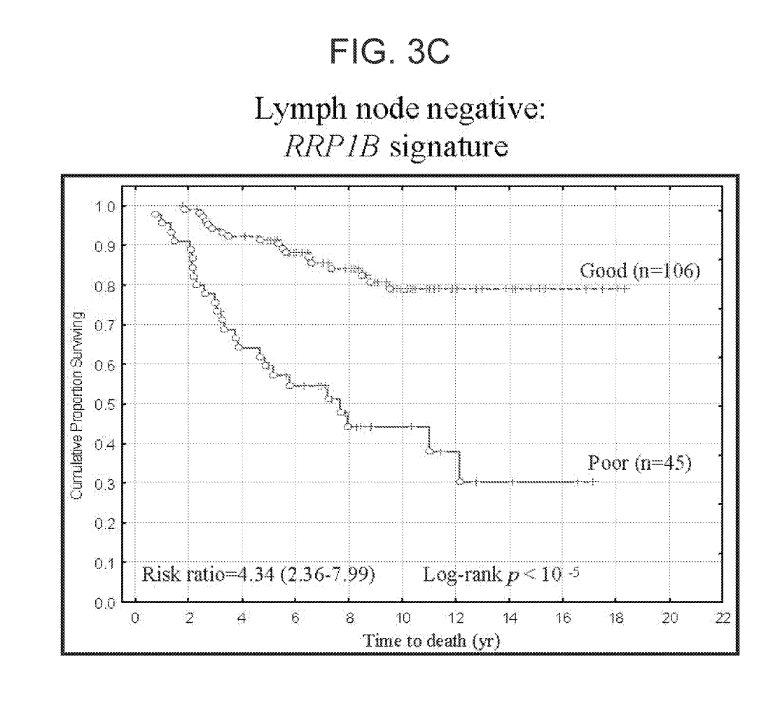

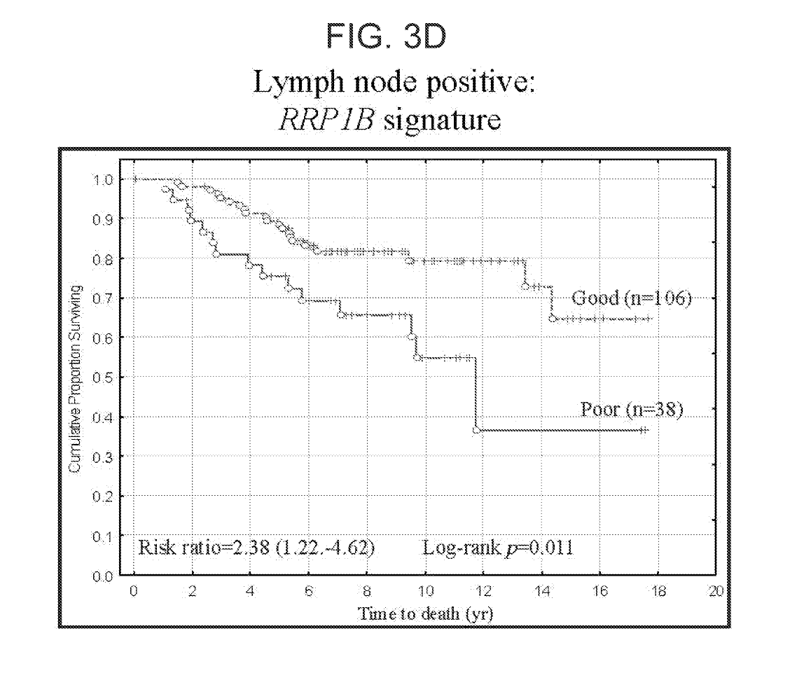

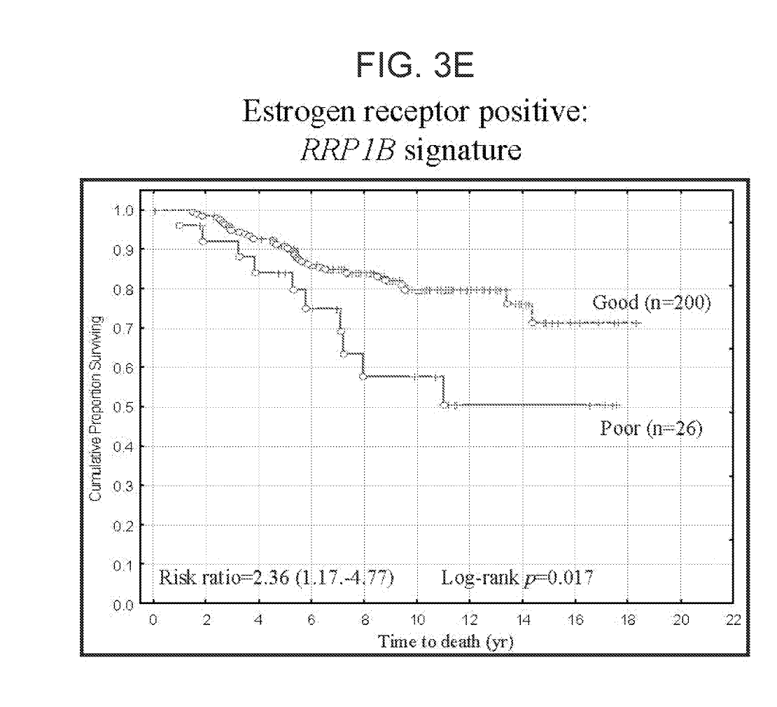

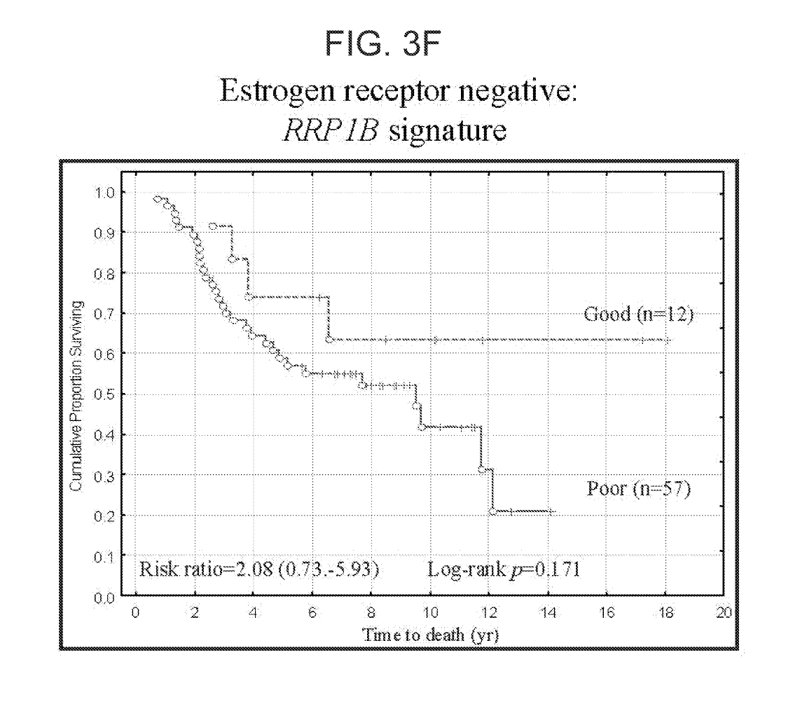

Kaplan-Meier survival analysis is performed to investigate whether there is a survival difference between groups. A significant survival difference is observed implying that the level of activation of Anakin or Anakin-associated pathways within a tumor, presumably because of either somatic mutation or germline polymorphism, is an important determinant of the overall likelihood of relapse and/or survival (FIG. 3A). Further analysis indicates that survival is associated primarily because of the effects of thirty-three genes (which genes form Group 6 as indicated in Table 1). The degree of survival difference represented by the 33-gene Anakin-induced gene expression signature is similar to the original 70-gene signature described by van't Veer and colleagues (van't Veer et al., 2002, supra) (FIG. 3B).

Patient samples are stratified by estrogen receptor (ER) and lymph node (LN) status, two clinically relevant prognostic markers, to determine whether the Anakin signature might provide additional clinical stratification. Expression of the Anakin signature in bulk primary tumor tissue predicts outcome in both LN negative and LN positive patients and patients with ER positive tumors (FIGS. 3C, 3D & 3E, respectively). ER negative patients do not show a significant survival benefit (FIG. 3F). However, this may be due to the limited sample size and needs to be clarified with additional studies.

This example demonstrated the generation of a human Anakin gene expression signature and further suggests its relevance as a diagnostic and prognostic tool.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An array comprising a substrate and a set of addressable elements, wherein each addressable element comprises (i) a polynucleotide that specifically binds to a target molecule, (ii) a polypeptide that specifically binds to a target molecule, or (iii) a combination of (i) and (ii), wherein the target molecule is selected from the group consisting of the target molecules listed in Table 1, wherein the array comprises less than 38,500 addressable elements, wherein, when the array is specific for the target molecules in Table 3, the array is specific for at least one target molecule listed in Table 1 that is not listed in Table 3.

2. The array of claim 1, comprising less than about 33,000 addressable elements.

3. The array of claim 2, comprising less than about 14,500 addressable elements.

4. The array of claim 3, comprising less than about 8400 addressable elements.

5. The array of claim 4, comprising less than about 5000 addressable elements.

6. The array of claim 1, wherein the set of addressable elements is specific for one or more of the target molecules of any of Groups 1 to 4, or a combination thereof.

7. The array of claim 1, wherein the set consists essentially of addressable elements specific for the target molecules of Table 1 or of any of Groups 1 to 4, or a combination thereof.

8. An array comprising a substrate and a set of addressable elements, wherein each addressable element comprises (i) a polynucleotide that specifically binds to a target molecule, (ii) a polypeptide that specifically binds to a target molecule, or (iii) a combination of (i) and (ii), wherein the target molecule is selected from the group consisting of the target molecules listed in Table 2, wherein the array comprises less than 38,500 addressable elements, wherein, when the array is specific for the target molecules in Table 3, the array is specific for at least one target molecule listed in Table 2 that is not listed in Table 3.

9. The array of claim 8, comprising less than about 33,000 addressable elements.

10. The array of claim 9, comprising less than about 14,500 addressable elements.

11. The array of claim 10, comprising less than about 8400 addressable elements.

12. The array of claim 11, comprising less than about 5000 addressable elements.

13. The array of claim 8, wherein the set of addressable elements is specific for one or more of the molecules of any of Groups 5 to 9, or a combination thereof.

14. The array of claim 8, wherein the set consists of addressable elements specific for one or more of the target molecules of Table 2 or of any of Groups 5 to 9, or a combination thereof.

15. A kit comprising a set of user instructions and (i) a set of polynucleotides, (ii) a set of polypeptides, or (iii) a combination of (i) and (ii), wherein the set of polynucleotides is specific for one or more of the target molecules listed in Table 1, wherein the set of polypeptides is specific for one or more of the target molecules listed in Table 1, wherein the kit is specific for less than 38,500 target molecules, wherein, when the kit is specific for the target molecules in Table 3, the kit is specific for at least one target molecule listed in Table 1 that is not listed in Table 3.

16. A kit comprising a set of user instructions and (i) a set of polynucleotides, (ii) a set of polypeptides, or (iii) a combination thereof, wherein the set of polynucleotides is specific for one or more of the target molecules listed in any of Table 2, wherein the set of polypeptides is specific for one or more of the target molecules listed in any of Table 2, wherein the kit is specific for less than 38,500 target molecules, wherein, when the kit is specific for the target molecules in Table 3, the kit is specific for at least one target molecule listed in Table 2 that is not listed in Table 3.

17. A method of characterizing a tumor or cancer in a subject comprising (i) detecting the expression levels of a set of target molecules in the subject, wherein the set of target molecules comprises one or more of the target molecules listed in Table 1 or 2, or any of Groups 1 to 9, or a combination thereof, wherein the expression levels are detected with the array of claim 1.

18. The method of claim 17, wherein the set of target molecules consists of all the target molecules of any of Groups 1 to 9 or a combination thereof.

19. A method of characterizing a tumor or cancer in a subject comprising (i) detecting the expression levels of a set of target molecules in the subject, wherein the set of target molecules consists of all the target molecules listed in Table 1 or 2, or any of Groups 1 to 9, or a combination thereof, and (ii) comparing the expression levels of the set of target molecules to a control set of expression levels.

20. The array of claim 17, wherein the method characterizes the tumor or cancer in terms of metastatic capacity, tumor stage, nodal involvement, regional metastasis, distant metastasis, tumor size, and/or sex hormone receptor status.

21. The array of claim 17, further comprising predicting whether the subject will survive from the cancer.

22. The array of claim 17, further comprising determining a treatment for the subject.

23. The array of claim 17, wherein the cancer is an epithelial cancer.

24. The method of claim 23, wherein the cancer is breast cancer.

25. The array of claim 17, wherein the subject is Swedish, Dutch, or Singaporean.

26-27. (canceled)

28. A method for treating cancer in a subject comprising:
(a) obtaining a sample from the subject;
(b) preparing the sample and applying the sample to the array of claim 1;
(c) determining the expression levels of a set of target molecules, wherein the set of target molecules comprises one or more of the target molecules listed in Table 1 or 2; and
(d) administering to the subject a compound with anti-cancer activity based on the expression levels determined in (c).

29. A method for treating cancer in a subject comprising:
(a) obtaining a sample from the subject;
(b) preparing the sample and applying the sample to the array of claim 1;
(c) determining the expression levels of a set of target molecules, wherein the set of target molecules consists of the target molecules listed in any of Table 1 or 2, or a combination thereof; and
(d) administering to the subject a compound with anti-cancer activity based on the expression levels determined in (c).

30. A method for treating cancer in a subject comprising:
(a) obtaining a sample from the subject;
(b) preparing the sample and applying the sample to the kit of claim 15;
(c) determining the expression levels of a set of target molecules, wherein the set of target molecules comprises one or more of the target molecules listed in Table 1 or 2; and
(d) administering to the subject a compound with anti-cancer activity based on the expression levels determined in (c).
